(19) United States
(12) Patent Application Publication
(10) Pub. No.: US 2025/0013150 A1
(43) Pub. Date: Jan. 9, 2025

(54) RADIATION-SENSITIVE COMPOSITION, PATTERN FORMATION METHOD, AND PHOTO-DEGRADABLE BASE

(71) Applicant: JSR CORPORATION, Tokyo (JP)

(72) Inventors: Ryuichi NEMOTO, Tokyo (JP); Kota FURUICHI, Tokyo (JP); Ryosuke NAKAMURA, Tokyo (JP); Tsuyoshi FURUKAWA, Tokyo (JP)

(73) Assignee: JSR CORPORATION, Tokyo (JP)

(21) Appl. No.: 18/882,982

(22) Filed: Sep. 12, 2024

Related U.S. Application Data
(63) Continuation of application No. PCT/JP2023/009713, filed on Mar. 13, 2023.

(30) Foreign Application Priority Data
Mar. 31, 2022 (JP) 2022-060693
Sep. 28, 2022 (JP) 2022-155404

(51) Int. Cl. G03F 7/004; C07C 69/75; C07C 309/12; C07C 309/17; C07C 309/27; C07D 207/416; C07D 307/00; C07D 307/94; C07D 311/00; C07D 313/06; C07D 321/06; C07D 327/04; C07D 327/06; G03F 7/039; G03F 7/32

(52) U.S. Cl. CPC G03F 7/0045; C07C 69/75; C07C 309/12; C07C 309/17; C07C 309/27; C07D 207/416; C07D 307/00; C07D 307/94; C07D 311/00; C07D 313/06; C07D 321/06; C07D 327/04; C07D 327/06; G03F 7/0395; G03F 7/32; C07C 2601/14; C07C 2602/20; C07C 2603/74

(57) ABSTRACT

A radiation-sensitive composition contains a polymer having an acid-releasable group, and a compound (Q) represented by formula (1). In the formula (1), L¹ represents an ester group, —CO—NR³—, a (thio)ether group, or a sulfonyl group. R⁴ represents a hydrogen atom, a substituted or unsubstituted C1 to C20 monovalent hydrocarbon group, a halogen atom, a hydroxy group, or a nitro group. R⁵ represents a C1 to C20 monovalent hydrocarbon group, a C1 to C20 monovalent halogenated hydrocarbon group, or a halogen atom, and optionally two R⁵s taken together represent an alicyclic structure together with the carbon atom(s) between the two R⁵s. L² represents a single bond or a divalent linking group.

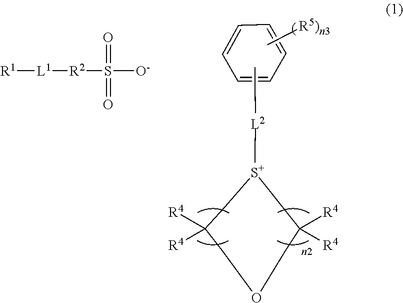

### CROSS-REFERENCE TO RELATED APPLICATIONS

The present application is a continuation application of International Patent Application No. PCT/JP2023/009713 filed Mar. 13, 2024, which claims priority to Japanese Patent Application No. 2022-60693 filed Mar. 31, 2022, and to Japanese Patent Application No. 2022-155404 filed Sep. 28, 2022. The disclosures of these applications are incorporated herein by reference.

### BACKGROUND OF THE DISCLOSURE

### Technical Field

The present disclosure relates to a radiation-sensitive composition, to a pattern formation method, and to a light-degradable base.

### Discussion of the Background

Photolithographic techniques making use of a resist composition are employed for forming microcircuits of semiconductor elements. In a typical mode of a photolithographic technique, firstly, a film formed from a resist composition (hereinafter may also be referred to as a “resist film”) is exposed to radiation through a mask pattern. Through exposure to the radiation, an acid is generated, and a chemical reaction involving the acid is evoked, to thereby provide a difference in dissolution rate to a developer between the light-exposed part and the light-unexposed part in the resist film. Subsequently, a developer is caused to come into contact with the resist film, to thereby form a resist pattern on a substrate.

For example, Japanese Patent Application Laid-Open (kokai) No. 2014-167611 discloses a resist composition which contains a resin having an acid-unstable group, and an onium salt serving as an acid-generating agent, the onium salt being formed of a thioxane-type sulfonium cation and a sulfonate anion having a particular structure.

### SUMMARY

According to an aspect of the present disclosure, a radiation-sensitive composition including: a polymer including an acid-releasable group; and a compound (Q) represented by formula (1).

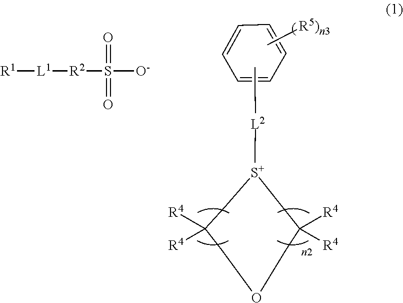

L¹ represents an ester group, —CO—NR³—, a (thio)ether group, or a sulfonyl group; when L¹ is an ester group, a (thio)ether group, or a sulfonyl group, R¹, R², and R³ satisfy condition (i) or (ii); when L¹ is —CO—NR³—, R¹, R², and R³ satisfy condition (i), (ii), or (iii); R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom, and optionally two R⁵s taken together represent an alicyclic structure together with the carbon atom(s) between the two R⁵s; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another:
(i) R¹ represents a C1 to C20 monovalent organic group which bound to L¹ via a carbon atom included in R¹; R² represents a substituted or unsubstituted divalent hydrocarbon group, comprising no fluorine atom; and R³ represents a hydrogen atom or a monovalent hydrocarbon group;
(ii) R¹ and R² taken together represent a group comprising an aliphatic heterocyclic structure together with L¹ to which R¹ and R² are bound, provided that R² comprises no fluorine atom; and R³ represents a hydrogen atom or a monovalent hydrocarbon group; and
(iii) R¹ represents a C1 to C20 monovalent organic group which bound to L¹ via a carbon atom included in R¹; R² and R³ taken together represent an aliphatic heterocyclic structure together with L¹ to which R² and R³ are bound, the aliphatic heterocyclic structure comprising no fluorine atom.

According to another aspect of the present disclosure, a pattern formation method, includes: forming a resist film by applying the radiation-sensitive composition according to claim onto a substrate, exposing the resist film to a radiation, and developing the radiation-exposed resist film.

According to a further aspect of the present disclosure, a light-degradable base is represented by formula (1).

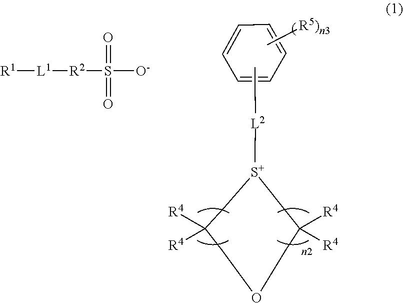

L¹ represents an ester group, —CO—NR³—, a (thio)ether group, or a sulfonyl group; when L¹ is an ester group, a (thio)ether group, or a sulfonyl group, R¹, R², and R³ satisfy the condition (i) or (ii); when L¹ is —CO—NR³—, R¹, R², and R³ satisfy the condition (i), (ii), or (iii); R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom, and optionally two R⁵s taken together represent an alicyclic structure together with the carbon atom(s) between the two R⁵s; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another:
(i) R¹ represents a C1 to C20 monovalent organic group which bound to L¹ via a carbon atom included in R¹; R² represents a substituted or unsubstituted divalent hydrocarbon group, comprising no fluorine atom; and R³ represents a hydrogen atom or a monovalent hydrocarbon group;
(ii) R¹ and R² taken together represent an aliphatic heterocyclic structure together with L¹ to which R¹ and R² are bound, provided that R² comprises no fluorine atom; and R³ represents a hydrogen atom or a monovalent hydrocarbon group; and
(iii) R¹ represents a C1 to C20 monovalent organic group which bound to L¹ via a carbon atom included in R¹; R² and R³ taken together represent an aliphatic heterocyclic structure together with L¹ to which R² and R³ are bound, the aliphatic heterocyclic structure comprising no fluorine atom.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

In photolithographic techniques employing a resist composition, dimensional reduction of patterns is proceeded by employing a short-wavelength radiation such as an ArF excimer beam, liquid immersion lithography (i.e., conducting light exposure while a space between the lens of an exposure apparatus and a resist film is filled with a liquid medium), or the like. As a next-generation technique, there has also been investigated a lithographic technique employing radiation of a further shorter wavelength (e.g., an electron beam, an X-ray, or an extreme ultraviolet ray (EUV). In such an attempt to establish a next-generation technique, there is demand for lithographic performance at least equal to conventionally attained performance, in terms of radiation sensitivity of a resist composition; line width roughness (LWR) performance (i.e., an index for variation in line width of a resist pattern); critical dimension uniformity (CDU) performance (i.e., an index for uniformity in line width and hole diameter), and pattern rectangularity (showing rectangularity of a cross-sectional shape of a resist pattern), and the like.

In the case where the radiation-sensitive acid-generator incorporated into a resist composition is easy to be degraded, the sensitivity and lithographic performance of the resist composition may possibly decrease during a long-term storage. Thus, the resist composition also requires to have suitable storage stability.

One embodiment of the present disclosure provides a radiation-sensitive composition which contains a polymer having an acid-releasable group, and a compound (Q) represented by formula (1).

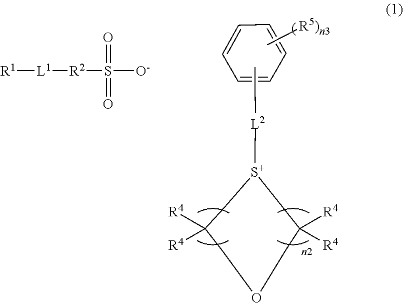

In the formula (1), L¹ represents an ester group, —CO—NR³—, a (thio)ether group, or a sulfonyl group; when L¹ is an ester group, a (thio)ether group, or a sulfonyl group, R¹, R², and R³ satisfy the condition (i) or (ii); when L¹ is —CO—NR³—, R¹, R², and R³ satisfy the condition (i), (ii), or (iii);
(i) R¹ represents a C1 to C20 monovalent organic group which bound to L¹ via a carbon atom; R² represents a substituted or unsubstituted divalent hydrocarbon group but contains no fluorine atom; and R³ represents a hydrogen atom or a monovalent hydrocarbon group;
(ii) R¹ and R² form a group including an aliphatic heterocyclic structure formed by combining R¹ and R² with each other together with L¹ to which R¹ and R² are bound, but R² contains no fluorine atom; and R³ represents a hydrogen atom or a monovalent hydrocarbon group; and
(iii) R¹ represents a C1 to C20 monovalent organic group which bound to L¹ via a carbon atom; R² and R³ form an aliphatic heterocyclic structure formed by combining R² and R³ with each other together with L¹ to which R² and R³ are bound, but the aliphatic heterocyclic structure contains no fluorine atom;
R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom; two R⁵s are combined with each other to form an alicyclic structure together with the carbon atoms to which the R⁵s are bound; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another.

Another embodiment of the present disclosure provides a pattern formation method (hereinafter may also be referred to as a “pattern formation method”), including forming a resist film by applying the aforementioned radiation-sensitive composition on a substrate, exposing the resist film to a radiation, and developing the radiation-exposed resist film.

Still another embodiment of the present disclosure provides a light-degradable base represented by the aforementioned formula (1).

The radiation-sensitive composition of the present disclosure, containing a polymer having an acid-releasable group and a compound (Q) represented by the aforementioned formula (1), can exhibit high sensitivity and suitable storage stability, and excellent LWR performance, CDU performance, and pattern rectangularity in formation of a resist pattern. According to the pattern formation method of the present disclosure, the radiation-sensitive composition of the present disclosure is used, whereby high precision and quality of a fine resist pattern can be further enhanced.

Hereinafter, carrying out of the present disclosure will be described in detail. In the present specification, the numerical range described with “A to B” refers to include “A” as a lower limit value and “B” as an upper limit value.

### <<Radiation-Sensitive Composition>>

The radiation-sensitive composition of the present disclosure (hereinafter may also be referred to as “the present composition”) contains a polymer having an acid-releasable group (hereinafter may also be referred to as a “polymer (A)”) and a compound (Q) having a particular structure. Also, so long as the effects of the present disclosure are not impaired, the present composition may contain a component differing from the polymer (A) and the compound (Q) (hereinafter may also referred to as an “optional component”). The components will next be described in detail.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is composed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure. The term “organic group” refers to an atomic group formed by removing any hydrogen atom from a carbon-containing compound (i.e., an organic compound). The term “(meth)acryl” collectively refers to “acryl” and “methacryl.” The term “(thio)ether” collectively refers to “ether” and “thio ether.”

### <Polymer (A)>

The acid-releasable group which is present in the polymer (A) is a group which can substitute a hydrogen atom of an acidic group (e.g., a carboxy group, a phenolic hydroxy group, an alcoholic hydroxy group, or a sulfo group) and which is released by the action of acid. By incorporating the polymer having an acid-releasable group into the radiation-sensitive composition, the acid-releasable group is released through a chemical reaction involving an acid generated through exposure to light, to thereby generate an acidic group. The acid-releasable group modifies the solubility of the polymer in a developer. As a result, excellent lithographic characteristics can be imparted to the present composition.

The polymer (A) preferably includes a structural unit (i.e., a constitutional unit) having an acid-releasable group (hereinafter may also be referred to as a “structural unit (I)”). Examples of the structural unit (I) include a structural unit having a structure in which the hydrogen atom of a carboxy group is substituted by a substituted or unsubstituted tertiary hydrocarbon group; a structural unit having a structure in which the hydrogen atom of a phenolic hydroxy group is substituted by a substituted or unsubstituted tertiary hydrocarbon group; and a structural unit having an acetal structure. From the viewpoint of enhancing the pattern rectangularity of the present composition, among the structural units (I), a structural unit having a structure in which the hydrogen atom of a carboxy group is substituted by a substituted or unsubstituted tertiary hydrocarbon group is preferred. More specifically, the structural units represented by the following formula (3) (hereinafter may also be referred to as a “structural unit (I-1)”) is preferred.

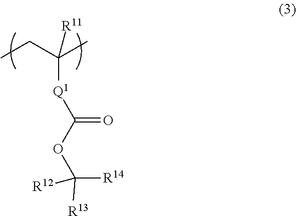

In the formula (3), R¹¹ represents a hydrogen atom, a fluorine atom, a methyl group, a trifluoromethyl group, or an alkoxyalkyl group; Q¹ represents a single bond or a substituted or unsubstituted divalent hydrocarbon group; R¹² represents a C1 to C20 substituted or unsubstituted monovalent hydrocarbon group; and each of R¹³ and R¹⁴ independently represents a C1 to C10 monovalent chain hydrocarbon group or a C3 to C20 monovalent alicyclic hydrocarbon group, or R¹³ and R¹⁴ are combined with each other and form, together with the carbon atom to which the R¹³ and R¹⁴ are bound, a C3 to C20 divalent alicyclic hydrocarbon group.

In the formula (3), from the viewpoint of co-polymerizability of a monomer forming the structural unit (I-1), R¹¹ is preferably a hydrogen atom or a methyl group, more preferably a methyl group. The divalent hydrocarbon group represented by Q¹ is preferably a divalent aromatic ring group, a phenylene group, or a naphthanylene group. When Q¹ is a substituted divalent hydrocarbon group, examples of the substituent include a halogen atom (e.g., a fluorine atom).

Examples of the C1 to C20 monovalent hydrocarbon group represented by R¹² include a C1 to C10 monovalent chain hydrocarbon group, a C3 to C20 monovalent alicyclic hydrocarbon group, and a C6 to C20 monovalent aromatic hydrocarbon group. When R¹² is a substituted monovalent hydrocarbon group, examples of the substituent include a halogen atom (e.g., a fluorine atom) and an alkoxy group.

Examples of the C1 to C10 monovalent chain hydrocarbon group represented by any of R¹² to R¹⁴ include a C1 to C10 linear-chain or branched saturated hydrocarbon group and a C1 to C10 linear-chain or branched unsaturated hydrocarbon group. Among them, the C1 to C10 monovalent chain hydrocarbon group represented by any of R¹² to R¹⁴ is preferably a C1 to C10 linear-chain or branched saturated hydrocarbon group.

Examples of the C3 to C20 monovalent alicyclic hydrocarbon group represented by any of R¹² to R¹⁴ include a C3 to C20 monocyclic saturated alicyclic hydrocarbon, a C3 to C20 monocyclic unsaturated alicyclic hydrocarbon, and a group formed by removing one hydrogen atom from a C3 to C20 alicyclic polycyclic hydrocarbon. Specific examples of such an alicyclic hydrocarbon include monocyclic saturated alicyclic hydrocarbons such as cyclobutane, cyclopentane, cyclohexane, cycloheptane, and cyclooctane; monocyclic unsaturated alicyclic hydrocarbons such as cyclopentene, cyclohexene, cycloheptene, cyclooctene, and cyclodecene; and polycyclic alicyclic hydrocarbons such as bicyclo[2.2.1]heptane (norbornane), bicyclo[2.2.2]octane, tircyclo[3.3.1.1^{3,7}]decane (adamantane), and tetracyclo[6.2.1.1^{3,6}.0^{2,7}]dodecane.

Examples of the C6 to C20 monovalent aromatic hydrocarbon group represented by R¹² include a group formed by removing one hydrogen atom from an aromatic ring such as benzene, naphthalene, anthracene, indene, or fluorene.

From the viewpoints of sufficiently removing development residue and enhancing the solution contrast to a developer between the light-exposed part and the light-unexposed part, R¹² is preferably, among others, a C1 to C8 substituted or unsubstituted monovalent hydrocarbon group, more preferably a C1 to C8 linear-chain or branched monovalent saturated hydrocarbon group or a C3 to C8 monovalent alicyclic hydrocarbon group.

Examples of the C3 to C20 divalent alicyclic hydrocarbon group formed by combining R¹³ and R¹⁴ with each other together with the carbon atom to which the R¹³ and R¹⁴ are bound include a group formed by removing two hydrogen atoms from each of the common carbon atom a carbon ring of any of the aforementioned monocyclic or polycyclic alicyclic hydrocarbon having the aforementioned number of carbon atoms. The divalent alicyclic hydrocarbon group formed by combining R¹³ and R¹⁴ may be a monocyclic hydrocarbon group or a polycyclic hydrocarbon group. When the divalent alicyclic hydrocarbon group formed by combining R¹³ and R¹⁴ is a polycyclic hydrocarbon group, the polycyclic hydrocarbon group may be a bridged alicyclic hydrocarbon group or a condensed alicyclic hydrocarbon group. Alternatively, the polycyclic hydrocarbon group may be a saturated hydrocarbon group or an unsaturated hydrocarbon group. The polycyclic hydrocarbon group preferably a saturated hydrocarbon group.

As used herein, the term “bridged alicyclic hydrocarbon” refers to a polycyclic alicyclic hydrocarbon in which two carbon atoms selected from the carbon atoms forming the alicycles and not being adjacent to each other are linked by the mediation of a bond linkage chain having one or more carbon atoms. The term “condensed alicyclic hydrocarbon” refers to a polycyclic alicyclic hydrocarbon in which a plurality of alicycles possess a common side (i.e., a bond between two carbon atoms adjacent to each other). The term “spiro ring hydrocarbon” refers to a polycyclic hydrocarbon in which two rings include a common atom. The spiro ring hydrocarbon may be a combination of monocyclic structures or may include a bridged structure or a condensed cyclic structure. The “alicyclic polycyclic hydrocarbon” encompasses a bridged alicyclic hydrocarbon, a condensed alicyclic hydrocarbon, and a spiro ring hydrocarbon.

Among the monocyclic alicyclic hydrocarbon groups (hereinafter may also be referred to as “monocyclic aliphatic hydrocarbon groups”), the saturated hydrocarbon group is preferably a cyclopentanediyl group, a cyclohexanediyl group, a cycloheptanediyl group, or a cyclooctanediyl group. The unsaturated hydrocarbon group is preferably a cyclopentenediyl group, a cyclohexenediyl group, a cycloheptenediyl group, or a cyclooctenediyl group. The polycyclic alicyclic hydrocarbon group (hereinafter may also be referred to as a “polycyclic aliphatic hydrocarbon group”) is preferably a bridged alicyclic saturated hydrocarbon group, with a bicyclo[2.2.1]heptane-2,2-diyl group (norbornane-2,2-diyl group), a bicyclo[2.2.2]octane-2,2-diyl group, a tetracyclo[6.2.1.1^{3,6}.0^{2,7}]dodecanediyl group, or a tricyclo[3.3.1.1^{3,7}]decane-2,2-diyl group (adamantane-2,2-diyl group) being more preferred.

From the viewpoints of polymer (A) easily adjusting solubility in a developer, easily forming a micropattern, suppressing remaining of a compound derived from a released group in the film, to thereby suppress an increase in roughness, and achieving clear dissolution at the interface with the developer, the polymer (A) preferably includes, in at least a part of the structural unit (I), a structural unit represented by the following formula (4).

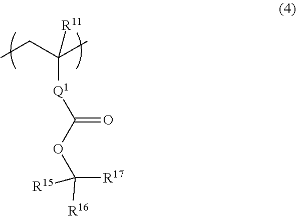

In the formula (4), R¹¹ represents a hydrogen atom, a fluorine atom, a methyl group, a trifluoromethyl group, or an alkoxyalkyl group; Q¹ represents a single bond or a substituted or unsubstituted divalent hydrocarbon group; R¹⁵ represents a C1 to C8 monovalent substituted or unsubstituted hydrocarbon group; and each of R¹⁶ and R¹⁷ independently represents a C1 to C8 monovalent chain hydrocarbon group or a C3 to C8 monovalent monocyclic aliphatic hydrocarbon group, or R¹⁶ and R¹⁷ are combined with each other and form, together with the carbon atom to which the R¹⁶ and R¹⁷ are bound, a C3 to C8 divalent monocyclic aliphatic hydrocarbon group.

In the formula (4), from the viewpoint of co-polymerizability of a monomer forming the structural unit represented by the formula (4), R¹¹ is preferably a hydrogen atom or a methyl group, more preferably a methyl group. Specific examples of Q¹ include the same groups as exemplified in relation to Q¹ in the formula (3).

Specific examples of R¹⁵, R¹⁶, and R¹⁷ include those described in relation to R¹², R¹³, and R¹⁴ in the aforementioned formula (3) and corresponding to the same carbon number equivalents. Of these, R¹⁵ is preferably a C1 to C5 linear-chain or branched monovalent saturated chain hydrocarbon group or a C3 to C8 monovalent alicyclic hydrocarbon group, more preferably a C1 to C3 linear-chain or branched monovalent saturated chain hydrocarbon group or a C3 to C5 monovalent monocyclic aliphatic hydrocarbon group. Preferably, each of R¹⁶ and R¹⁷ represents a C1 to C4 linear-chain or branched monovalent chain saturated hydrocarbon group, or R¹⁶ and R¹⁷ are combined with each other and form, to together with the carbon atom to which R¹⁶ and R¹⁷ are bound, a C3 to C8 divalent monocyclic aliphatic hydrocarbon group.

Among the aforementioned structural units, the structural unit represented by the aforementioned formula (4) is particularly preferably a structural unit in which R¹⁵ is a C1 to C4 alkyl group, and R¹⁶ and R¹⁷ are combined with each other and form, together with the carbon atom to which R¹⁶ and R¹⁷ are bound, a C3 to C6 cycloalkanediyl group.

Also, from the viewpoint of enhancing etching resistance, the polymer (A) may include a structural unit represented by the following formula (5). From the viewpoints of enhancing solubility of the polymer (A), suppressing an increase in roughness, and enhancing etching resistance, the polymer (A) preferably includes a structural unit represented by the following formula (5) in addition to the structural unit represented by the aforementioned formula (4).

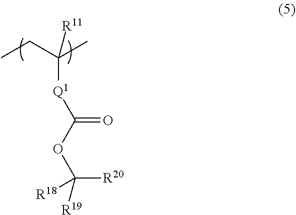

In the formula (5), R¹¹ represents a hydrogen atom, a fluorine atom, a methyl group, a trifluoromethyl group, or an alkoxyalkyl group; Q¹ represents a single bond or a substituted or unsubstituted divalent hydrocarbon group; R¹⁸ represents a C1 to C20 substituted or unsubstituted monovalent hydrocarbon group; regarding R¹⁹ and R²⁰, R¹⁹ represents a C1 to C10 monovalent chain hydrocarbon group or a C7 to C20 monovalent polycyclic aliphatic hydrocarbon group, and R²⁰ represents a C7 to C20 monovalent polycyclic aliphatic hydrocarbon group, or R¹⁹ and R²⁰ are combined with each other and form, together with the carbon atom to which the R¹⁹ and R²⁰ are bound, a C7 to C20 divalent polycyclic aliphatic hydrocarbon group.

In the formula (5), from the viewpoint of co-polymerizability of a monomer forming the structural unit represented by the formula (5), R¹¹ is preferably a hydrogen atom or a methyl group, more preferably a methyl group. Specific examples including preferred groups of Q¹ include the same groups as exemplified in relation to Q¹ in the formula (3).

Specific examples of R¹⁸, R¹⁹, and R²⁰ include those described in relation to R¹², R¹³, and R¹⁴ in the aforementioned formula (3) and corresponding to the same carbon number equivalents. Of these, R¹⁰ is preferably a C1 to C5 linear-chain or branched monovalent chain hydrocarbon group or a C3 to C10 monovalent alicyclic hydrocarbon group. Regarding R¹⁹ and R²⁰, preferably, R¹⁹ represents a C1 to C4 monovalent chain hydrocarbon group, and R²⁰ represents a C7 to C15 monovalent bridged aliphatic hydrocarbon group, or R¹⁹ and R²⁰ are combined with each other and form, together with the carbon atom to which the R¹⁹ and R²⁰ are bound, a C7 to C15 divalent bridged aliphatic hydrocarbon group.

Among the aforementioned examples, the structural unit represented by the aforementioned formula (5) is particularly preferably the following case, wherein each of R¹⁸ and R¹⁹ is a C1 to C4 alkyl group, and R²⁰ is a C7 to C15 saturated bridged alicyclic hydrocarbon group, or R¹⁸ is a C1 to C4 alkyl group, and R¹⁹ and R²⁰ are combined with each other and form, together with the carbon atom to which the R¹⁹ and R²⁰ are bound, a C7 to C15 saturated bridged alicyclic hydrocarbon group.

Specific examples of the structural unit (I) include structural units represented by the following formulas (3-1) to (3-7).

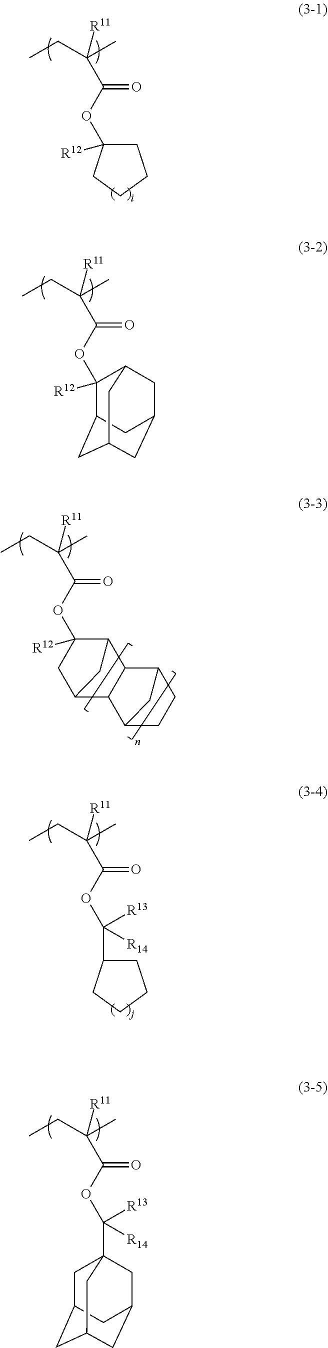

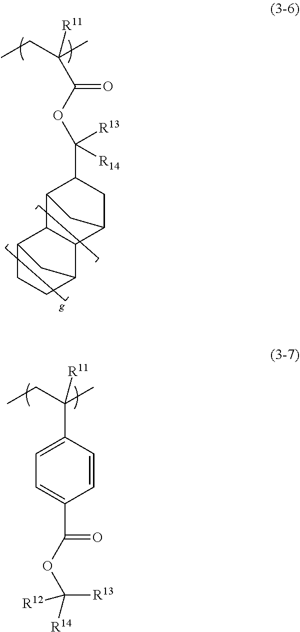

In the formulas (3-1) to (3-7), R¹¹ to R¹⁴ have the same meanings as defined in the aforementioned formula (3); each of i and j is independently an integer of 0 to 4; and each of h and g is independently 0 or 1.

In the formulas (3-1) to (3-7), each of i and j is preferably 1 or 2, more preferably 1. Each of h and g is preferably 1. R¹² is preferably a methyl group, an ethyl group, or an isopropyl group. Each of R¹³ and R¹⁴ is preferably a methyl group or an ethyl group.

The relative amount of the structural unit (I) in all the structural units forming the polymer (A) is preferably 15 mol % or more, more preferably 25 mol % or more, still more preferably 35 mol % or more. Also, the relative amount of the structural unit (I) in all the structural units forming the polymer (A) is preferably 80 mol % or less, more preferably 70 mol % or less, still more preferably 65 mol % or less. By adjusting the structural unit (I) content to satisfy the aforementioned conditions, LWR performance, CDU performance, and pattern rectangularity of the present composition can be further enhanced. Notably, the polymer (A) may include the structural unit (I) singly or in combination of two or more species.

When the polymer (A) include a structural unit represented by the aforementioned formula (4) as the structural unit (I), the relative amount of the structural unit represented by the aforementioned formula (4) in all the structural units forming the polymer (A) is preferably 10 mol % or more, more preferably 20 mol % or more, still more preferably 30 mol % or more. By adjusting the relative amount of the structural unit represented by the aforementioned formula (4) to satisfy the aforementioned conditions, solubility of the polymer (A) in a developer can be readily adjusted, and formation of a micropattern can be facilitated.

When the polymer (A) includes a structural unit represented by the aforementioned formula (5) as the structural unit (I), the relative amount of the structural unit represented by the aforementioned formula (5) in all the structural units forming the polymer (A) is preferably 1 mol % or more, more preferably 2 mol % or more, still more preferably 5 mol % or more. By adjusting the relative amount of the structural unit represented by the aforementioned formula (5) to satisfy the aforementioned conditions, the difference in dissolution rate to a developer between the light-exposed part and the light-unexposed part in the resist film formed from the present composition can increase, whereby the CDU performance and pattern rectangularity of the present composition can be enhanced. Also, when the polymer (A) includes a structural unit represented by the aforementioned formula (4) and a structural unit represented by the aforementioned formula (5), the relative amount of the structural unit represented by the aforementioned formula (5) in all the structural units forming the polymer (A) is preferably 30 mol % or less, more preferably 25 mol % or less, still more preferably 20 mol % or less.

### [Additional Structural Unit]

Along with the structural unit (I), the polymer (A) may further include a structural unit differing from the structural unit (I) (hereinafter may also be referred to as an “additional structural unit”). Examples of the additional structural unit include the following structural units (II) and (III).

### Structural Unit (II)

The polymer (A) may further include a structural unit having a polar group (hereinafter may also be referred to as a “structural unit (II)”). Through incorporation of the structural unit (II) into the polymer (A), solubility of the polymer (A) in a developer can be tuned in an easier manner, whereby lithographic performance such as resolution can be improved. Examples of the structural unit (II) include structural units having at least one structure selected from the group consisting of a lactone structure, a cyclic carbonate structure, and a sultone structure (hereinafter may also be referred to as a “structural unit (II-1)”) and a structural unit having a monovalent polar group (hereinafter may also be referred to as a “structural unit (II-2)”).

### Structural Unit (II-1)

Through incorporation of the structural unit (II-1) into the polymer (A), tuning of solubility of the polymer (A) in a developer, improvement of close adhesion of the resist film, and further enhancement of etching resistance can be achieved. Examples of the structural unit (II-1) include the structural units represented by the following formulas (6-1) to (6-10).

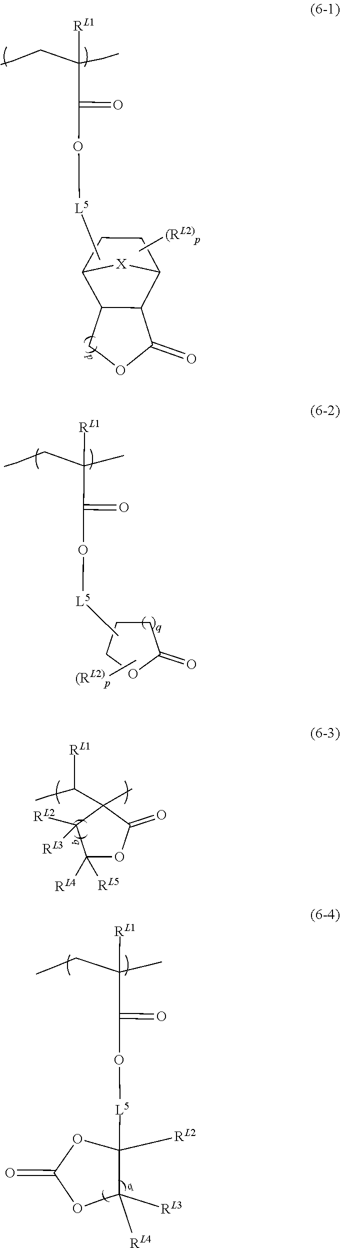

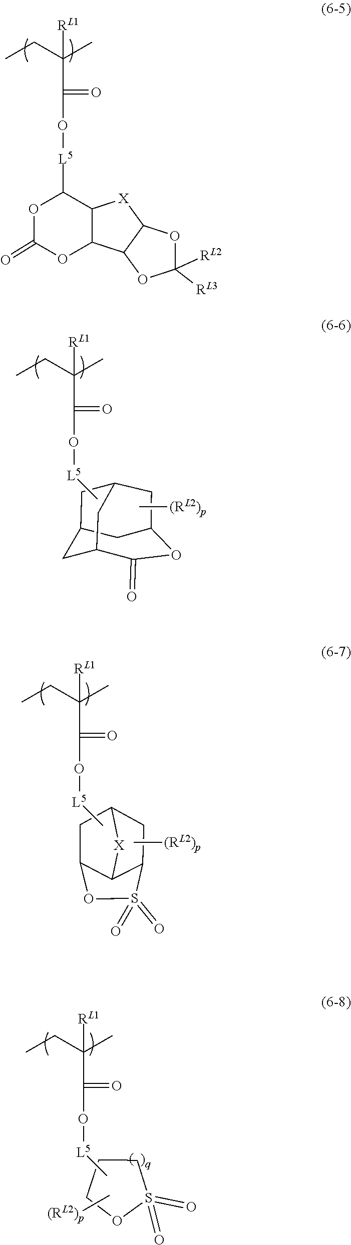

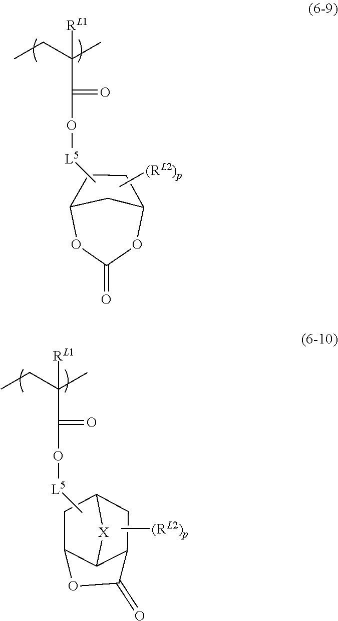

In the formulas (6-1) to (6-10), R^{L1} represents a hydrogen atom, a fluorine atom, a methyl group, a trifluoromethyl group, or an alkoxyalkyl group; each of R^{L2} and R^{L3} independently represents a hydrogen atom, a C1 to C4 alkyl group, a cyano group, a trifluoromethyl group, a methoxy group, a methoxycarbonyl group, a hydroxy group, a hydroxymethyl group, or a dimethylamino group; each of R^{L4} and R^{L5} independently represents a hydrogen atom, a C1 to C4 alkyl group, a cyano group, a trifluoromethyl group, a methoxy group, a methoxycarbonyl group, a hydroxy group, a hydroxymethyl group, or a dimethylamino group, or R^{L4} and R^{L5} are combined with each other and form, together with the carbon atom to which R^{L4} and R^{L5} are bound, a C3 to C8 divalent alicyclic hydrocarbon group; L⁵ represents a single bond or a divalent bonding group; X represents an oxygen atom or a methylene group; p is an integer of 0 to 3; and q is an integer of 1 to 3.

Examples of the C3 to C8 divalent alicyclic hydrocarbon group formed by combining R^{L4} and R^{L5} with each other and together with the carbon atom to which R^{L4} and R^{L5} are bound include those described in relation to R¹³ and R¹⁴ in the aforementioned formula (3) and corresponding to the same C3 to C8 equivalents. The one or more hydrogen atoms of the alicyclic hydrocarbon group may be substituted by a hydroxy group.

Examples of the divalent bonding group represented by L⁵ include a C1 to C10 linear-chain or branched divalent chain hydrocarbon group, a C4 to C12 divalent alicyclic hydrocarbon group, and a group formed from any one or more of the hydrocarbon groups and at least one of —CO—, —O—, —NH—, and —S—.

The structural unit (II-1) is preferably any of the structural units represented by formulas (6-2), (6-4), (6-6), (6-7), and (6-10), among those structural units represented by formulas (6-1) to (6-10).

When the polymer (A) includes the structural unit (II-1), the relative amount of the structural unit (II-1) in all the structural units forming the polymer (A) is preferably 80 mol % or less, more preferably 70 mol % or less, still more preferably 65 mol % or less. Also, when the polymer (A) includes the structural unit (II-1), the relative amount of the structural unit (II-1) in all the structural units forming the polymer (A) is preferably 2 mol % or more, more preferably 5 mol % or more, still more preferably 10 mol % or more. Through adjusting the structural unit (II-1) content to satisfy the aforementioned conditions, lithographic performance such as resolution of the present composition can be further enhanced.

Structural unit (II-2) In an alternative pathway, the structural unit (II-2) is incorporated into the polymer (A) so as to tune the solubility of the polymer (A) in a developer, whereby lithographic performance such as resolution of the present composition is enhanced. Examples of the polar group present in the structural unit (II-2) include a hydroxy group, a carboxy group, a cyano group, a nitro group, and a sulfonamide group. Among them, a hydroxy group and a carboxy group are preferred, with a hydroxy group (in particular, an alcoholic hydroxy group) being more preferred. Notably, the structural unit (II-2) is a structural unit differing from a structural unit having a phenolic hydroxy group (i.e., a structural unit (III) described hereinbelow).

As used herein, the term “phenolic hydroxy group” refers to a group in which a hydroxy group is directly bonded to an aromatic hydrocarbon structure. The term “alcoholic hydroxy group” refers to a group in which a hydroxy group is directly bonded to an aliphatic hydrocarbon structure. In the alcoholic hydroxy group, the aliphatic hydrocarbon structure to which a hydroxy group is bonded may be a chain hydrocarbon group or an alicyclic hydrocarbon group.

Examples of the structural unit (II-2) include the structural units represented by the following formulas. However, the structural unit (II-2) is not limited to the structures.

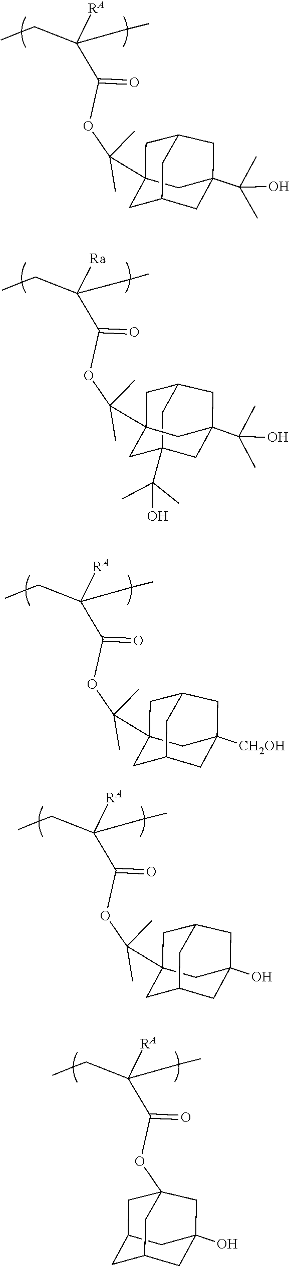

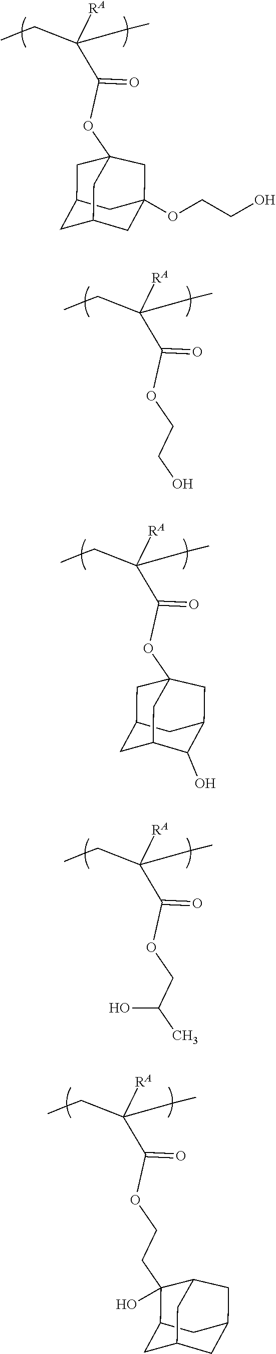

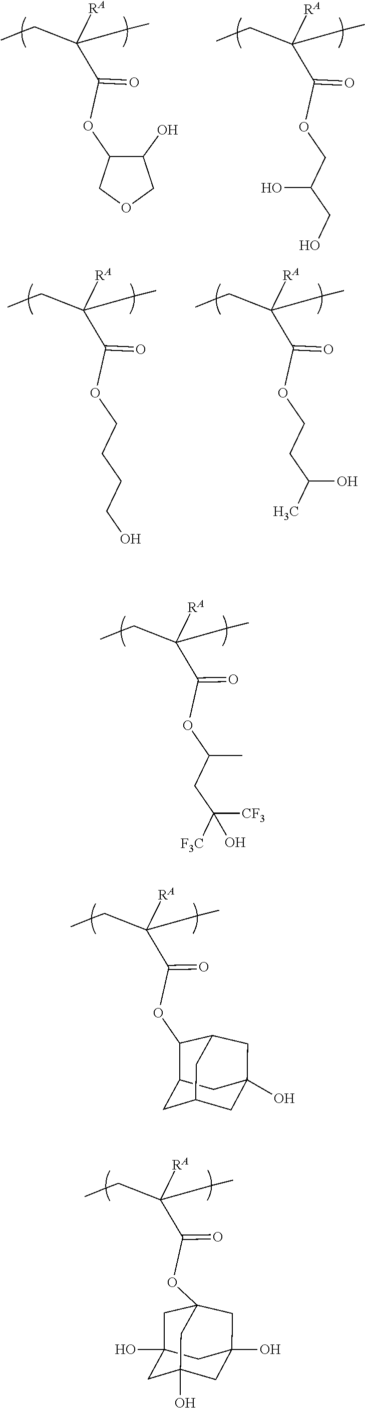

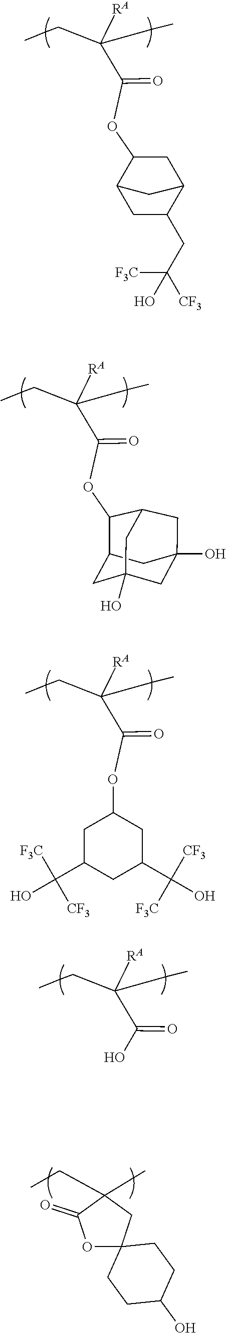

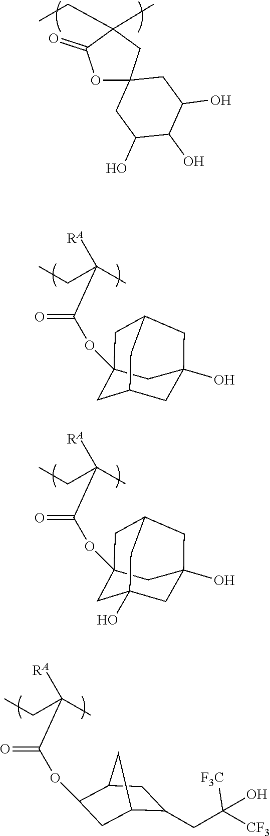

In the formulas, R^{A} represents a hydrogen atom, a fluorine atom, a methyl group, a trifluoromethyl group, or an alkoxyalkyl group.

When the polymer (A) includes the structural unit (II-2), the relative amount of the structural unit (II-2) in all the structural units forming the polymer (A) is preferably 2 mol % or more, more preferably 5 mol % or more. Also, the relative amount of the structural unit (II-2) in all the structural units forming the polymer (A) is preferably 30 mol % or less, more preferably 25 mol % or less, still more preferably 20 mol % or less. By adjusting the structural unit (II-2) content to satisfy the aforementioned conditions, lithographic performance such as resolution of the present composition can be further enhanced.

Structural unit (III) The polymer (A) may further include a structural unit having a phenolic hydroxy group (hereinafter may also be referred to as a “structural unit (III)”). The presence of the structural unit (III) in the polymer (A) is preferred, since etching resistance and difference in solubility in a developer between the light-exposed part and the light-unexposed part (i.e., dissolution contrast) can be enhanced.

Particularly when pattern formation is carried out through light exposure with radiation having a wavelength of 50 nm or shorter (e.g., electron beam or EUV), a polymer (A) including the structural unit (III) is preferably used. When the polymer (A) is applied to pattern formation through light exposure with radiation having a wavelength of 50 nm or shorter, the polymer (A) preferably includes the structural unit (III).

No particular limitation is imposed on the structural unit (III), so long as the unit has a phenolic hydroxy group. Specific examples of the structural unit (III) include a structural unit derived from hydroxystyrene or a derivative thereof, and a structural unit derived from a (meth)acrylic compound having a hydroxybenzene structure.

In the case where a polymer including the structural unit (III) is produced as the polymer (A), the structural unit (III) may be incorporated into the polymer (A) by conducting polymerization while a phenolic hydroxy group is protected by a protective group such as an alkali-releasable group during polymerization, and then conducting deprotection through hydrolysis. The structural unit providing the structural unit (III) through hydrolysis is preferably at least one species selected from the group consisting of the structural units represented by the following formulas (7-1) and (7-2).

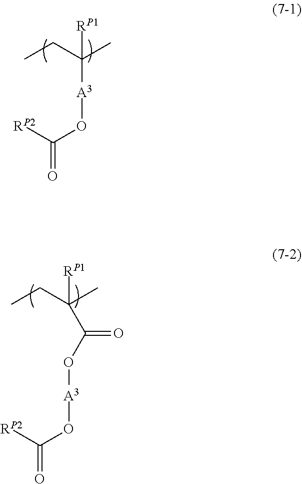

In the formulas (7-1) and (7-2), R^{P1} represents a hydrogen atom, a fluorine atom, a methyl group, a trifluoromethyl group, or an alkoxyalkyl group; A³ represents a substituted or unsubstituted divalent aromatic ring group; R^{P2} represents a C1 to C20 monovalent hydrocarbon group or an alkoxy group.

The aromatic ring group represented by A³ is a group formed by removing two hydrogen atoms from a ring moiety of the substituted or unsubstituted aromatic ring. The aromatic ring is preferably a hydrocarbon ring, and examples thereof include aromatic hydrocarbon rings such as benzene, naphthalene, and anthracene. Among them, A³ is preferably a group formed by removing two hydrogen atoms from a ring moiety of the substituted or unsubstituted benzene or naphthalene, more preferably a substituted or unsubstituted phenylene group. Examples of the substituent include a halogen atom such as a fluorine atom.

Examples of the C1 to C20 monovalent hydrocarbon group represented by R^{P2} include groups as exemplified in relation to the C1 to C20 monovalent hydrocarbon group of R¹² in the structural unit (I). Examples of the alkoxy group include a methoxy group, an ethoxy group, and a tert-butoxy group. Of these, R^{P2} is preferably an alkyl group or an alkoxy group, with a methyl group and a tert-butoxy group being particularly preferred.

When a radiation-sensitive composition for use in light exposure with radiation having a wavelength of 50 nm or shorter is prepared, the relative amount of the structural unit (III) in all the structural units forming the polymer (A) is preferably 15 mol % or more, more preferably 20 mol % or more. Also, the relative amount of the structural unit (III) in all the structural units forming the polymer (A) is preferably 65 mol % or less, more preferably 55 mol % or less.

Examples of the additional structural unit include, in addition to the aforementioned structural units, a structural unit derived from styrene, a structural unit derived from vinylnaphthalene, a structural unit derived from a monomer having an alicyclic structure (e.g., 1-adamantyl (meth)acrylate), and a structural unit derived from n-pentyl (meth)acrylate. The additional structural unit content may be appropriately set in a unit-by-unit manner, so long as the effects of the present disclosure are not impaired.

Synthesis of polymer (A) The polymer (A) may be synthesized through, for example, polymerization of monomers for providing the corresponding structural units in an appropriate solvent by use of a radical polymerization initiator or the like.

Examples of the radical polymerization initiator include azo-type radical initiators such as azobisisobutyronitrile (AIBN), 2,2′-azobis(4-methoxy-2,4-dimethylvaleronitrile), 2,2′-azobis(2-cyclopropylpropionitrile), 2,2′-azobis(2,4-dimethylvaleronitrile), and dimethyl 2,2′-azobisisobutyrate; and peroxide-type radical initiators such as benzoyl peroxide, t-butyl hydroperoxide, and cumene hydroperoxide. Of these, AIBN and dimethyl 2,2′-azobisisobutyrate are preferred, with AIBN being more preferred. These radical initiators may be used singly or in combination of two or more species.

Examples of the solvent employed in polymerization include an alkane, a cycloalkane, an aromatic hydrocarbon, a halogenated hydrocarbon, a saturated carboxylate ester, a ketone, an ether, and an alcohol. Specific examples of the alkane include n-pentane, n-hexane, n-heptane, n-octane, n-nonane, and n-decane. Specific examples of the cycloalkane include cyclohexane, cycloheptane, cyclooctane, decalin, and norbornane. Examples of the aromatic hydrocarbon include benzene, toluene, xylene, ethylbenzene, and cumene. Specific examples of the halogenated hydrocarbon include chlorobutane, bromohexane, dichloroethane, hexamethylene dibromide, and chlorobenzene. Specific examples of the saturated carboxylate ester include ethyl acetate, n-butyl acetate, i-butyl acetate, and methyl propionate. Specific examples of the ketone include acetone, methyl ethyl ketone, 4-methyl-2-pentanone, and 2-heptanone. Specific examples of the ether include tetrahydrofuran, dimethoxyethane, and diethoxyethane. Specific examples of the alcohol include methanol, ethanol, 1-propanol, 2-propanol, and 4-methyl-2-pentanol. These solvents employed in polymerization may be used singly or in combination of two or more species.

The reaction temperature in polymerization is generally 40° C. to 150° C., preferably 50° C. to 120° C. The reaction time is generally 1 hour to 48 hours, preferably 1 hour to 24 hours.

The weight average molecular weight (Mw) of the polymer (A), which is determined through gel permeation chromatography (GPC) and is reduced to polystyrene, is preferably 1,000 or more, more preferably 2,000 or more, still more preferably 3,000 or more, particularly preferably 4,000 or more. Also, the Mw of the polymer (A) is preferably 50,000 or less, more preferably 30,000 or less, still more preferably 20,000 or less, particularly preferably 15,000 or less. Adjusting the Mw of the polymer (A) so as to satisfy the above conditions is preferred, since coatability of the present composition and heat resistance of the formed resist film can be improved, and development failure can be sufficiently suppressed.

The ratio (Mw/Mn) of Mw to (Mn) of the polymer (A), which is determined through GPC and is reduced to polystyrene, is preferably 5.0 or less, more preferably 3.0 or less, still more preferably 2.0 or less. Also, the Mw/Mn of the polymer (A) is generally 1.0 or greater.

The polymer (A) content of the present composition, with respect to the total solid content of the present composition (i.e., the sum of the amounts by mass of the components other than the solvent), is preferably 70 mass % or more, more preferably 75 mass % or more, still more preferably 80 mass % or more. Also, the polymer (A) content of the present composition, with respect to the total solid content of the present composition, is preferably 99 mass % or less, more preferably 98 mass % or less, still more preferably 95 mass % or less. The polymer (A) preferably forms a base resin of the present composition. As used herein, the term “base resin” refers to a polymer component which accounts for ≥50 mass % of the total solid content of the present composition. The present composition may contain only one polymer (A) or two or more polymers (A).

### <Compound (Q)>

The compound (Q) is a compound represented by the following formula (1).

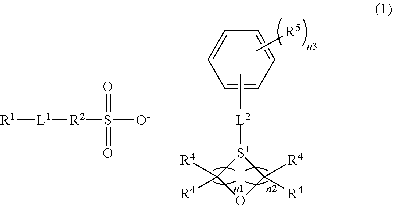

In the formula (1), L¹ represents an ester group, —CO—NR³—, a (thio)ether group, or a sulfonyl group; when L¹ is an ester group, a (thio)ether group, or a sulfonyl group, R¹, R², and R³ satisfy the condition (i) or (ii); when L¹ is —CO—NR³—, R¹, R², and R³ satisfy the condition (i), (ii), or (iii); (i) R¹ represents a C1 to C20 monovalent organic group which bound to L¹ via a carbon atom; R² represents a substituted or unsubstituted divalent hydrocarbon group but contains no fluorine atom; and R³ represents a hydrogen atom or a monovalent hydrocarbon group;
(ii) R¹ and R² form a group including an aliphatic heterocyclic structure formed by combining R¹ and R² with each other together with L¹, but R² contains no fluorine atom; and R³ represents a hydrogen atom or a monovalent hydrocarbon group; and
(iii) R¹ represents a C1 to C20 monovalent organic group which bound to L¹ via a carbon atom; R² and R³ form an aliphatic heterocyclic structure formed by combining R² and R³ with each other together L¹, but the aliphatic heterocyclic structure contains no fluorine atom; R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom; two R⁵s are combined with each other to form an alicyclic structure with the carbon atoms to which the R⁵s are bound; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another.

The compound (Q) can serve as a light-degradable base, which is a type of acid diffusion control agent. The light-degradable base is a component which suppresses diffusion of the acid generated through light exposure and originating from the acid-generating agent in the resist film, to thereby suppress chemical reaction caused by the acid in the light-unexposed part. The present composition, containing the polymer (A) and the compound (Q), exhibits high sensitivity and excellent LWR performance, CDU performance, and pattern rectangularity during formation of a resist pattern. Also, the compound (Q) is stable with time. Thus, the present composition containing the compound (Q) exhibits high sensitivity, excellent resist performance, and suitable storage stability.

The acid generated through exposing the light-degradable base to light is an acid that cannot evoke release of an acid-releasable group under generally employed conditions. The term “generally employed conditions” refers to carrying out post-exposure bake (PEB) at 110° C. for 60 seconds. In the light-unexposed part, the light-degradable base exhibits acid diffusion suppressing action by virtue of its basicity, whereas in the light-exposed part, a weak acid is generated from an anion and a proton generated through decomposition of a cation, whereby the acid diffusion suppressing action decreases. Thus, in the resist film containing a light-degradable base, the acid generated through light exposure efficiently works in the light-exposed part, to thereby release an acid-releasable group of the polymer (A). In contrast, variation of the components in the light-unexposed part of the resist film due to acid does not occurs. As a result, a more clear difference in solubility emerges between the light-exposed part and the light-unexposed part. Through incorporation of the compound (Q) into the present composition, high sensitivity, and excellent LWR performance, CDU performance, and pattern rectangularity can be achieved.

### Description of Anion

In the aforementioned formula (1), L¹ represents an ester group (—C(═O)—O—), an amide group (—C(═O)—NR³—), an ether group (—O—), a thioether group (—S—), or a sulfonyl group (—S(═O)₂—). However, no particular limitation is imposed on the direction of bonding of L¹. For example, when L¹ is an ester group, the carbonyl group forming the ester group may be bound to R¹ or R². From the viewpoint of ease of the synthesis of the compound (Q), L¹ is preferably, among others, an ester group or an amide group.

When L¹ is an ester group, a (thio)ether group, or a sulfonyl group, R¹, R², and R³ satisfy the condition (i) or (ii). When L¹ is an amide group, R¹, R², and R³ satisfy the condition (i), (ii), or (iii).

When R¹ is a C1 to C20 monovalent organic group which bound to L¹ via a carbon atom, the monovalent organic group represented by R¹ may be a group having a chain structure (i.e., a chain organic group) or a group having a cyclic structure (i.e., a cyclic organic group).

The expression “R¹ is bound to L¹ via a carbon atom” refers to that a carbonyl group, an oxygen atom, or a sulfur atom in L¹ is directly bound to a carbon atom in R¹. The carbon atom in R¹ to which the carbonyl group, the oxygen atom, or the sulfur atom in L¹ is bound may be a primary carbon atom, a secondary carbon atom, or a tertiary carbon atom. Also, carbon atom in R¹ to which the carbonyl group, the oxygen atom, or the sulfur atom in L¹ is bound may be located at a position adjacent to the oxygen atom or the carbonyl group in R¹.

When the monovalent organic group represented by R¹ is a chain organic group, examples of the chain organic group include a C1 to C20 linear-chain or branched saturated hydrocarbon group, a C1 to C20 linear-chain or branched unsaturated hydrocarbon group, a C2 to C20 monovalent group having a (thio)ether group or an ester group inserted into a carbon-carbon bond of the linear-chain or branched hydrocarbon group, and a C1 to C20 monovalent group in which any hydrogen atom of the linear-chain or branched hydrocarbon group is substituted. Examples of the substituent include a halogen atom (e.g., a fluorine atom, a chlorine atom, a bromine atom, or an iodine atom), a hydroxy group, and a nitro group.

When the monovalent organic group represented by R¹ is a cyclic organic group, examples of the cyclic structure in R¹ include a C3 to C20 alicyclic hydrocarbon structure, a C3 to C20 aliphatic heterocyclic structure, and a C6 to C20 aromatic ring structure. These cyclic structures may have a substituent. Examples of the substituent include an alkyl group, an alkoxy group, an alkoxycarbonyl group, an alkoxycarbonyloxy group, a halogen atom (e.g., a fluorine atom, a chlorine atom, a bromine atom, or an iodine atom), a hydroxy group, and an oxo group.

Examples of the C3 to C20 alicyclic hydrocarbon structure include a C3 to C20 alicyclic monocyclic structure and a C6 to C20 alicyclic polycyclic structure. The C3 to C20 alicyclic monocyclic structure and the C6 to C20 alicyclic polycyclic structure may be a saturated hydrocarbon structure or an unsaturated hydrocarbon structure. Also, the alicyclic polycyclic structure may be a bridged structure, a condensed ring structure, or a spiro ring structure. As used herein, the term “bridged structure” refers to a polycyclic structure in which two carbon atoms selected from the carbon atoms forming the alicycles and not being adjacent to each other are linked by the mediation of a bond linkage chain having one or more carbon atoms. The term “condensed cyclic structure” refers to a polycyclic structure in which a plurality of alicycles possess a common side (i.e., a bond between two carbon atoms adjacent to each other). The term “spiro ring structure” refers to a polycyclic structure in which two rings include a common atom. The spiro ring structure may be a combination of monocyclic structures or may include a bridged structure or a condensed ring structure.

Among the alicyclic monocyclic structures, examples of the saturated hydrocarbon structure include a cyclopentane structure, a cyclohexane structure, a cycloheptane structure, and a cyclooctane structure. Examples of the unsaturated hydrocarbon structure include a cyclopentene structure, a cyclohexene structure, a cycloheptene structure, a cyclooctene structure, and a cyclodecene structure. The alicyclic polycyclic structure is preferably a bridged alicyclic saturated hydrocarbon structure or a condensed alicyclic saturated hydrocarbon structure, and specific examples include a bicyclo[2.2.1]heptane structure, a bicyclo[2.2.2]octane structure, a tricyclo[3.3.1.1^{3,7}]decane structure, and a steroid structure.

Examples of the C3 to C20 aliphatic heterocyclic structure include a cyclic ether structure, a lactone structure, a cyclic acetal structure, a cyclic carbonate structure, and a sultone structure. The aliphatic heterocyclic structure may be a monocyclic structure or a polycyclic structure. The polycyclic structure may be a bridged structure, a condensed ring structure, or a spiro ring structure. The C3 to C20 aliphatic heterocyclic structure represented by R¹ may be a combination of two or more of the bridged structure, the condensed ring structure, and the spiro ring structure. When the C3 to C20 aliphatic heterocyclic structure represented by R¹ has a spiro ring structure, two or more rings forming the spiro ring structure may be formed of only aliphatic heterocycles, or a combination of an aliphatic heterocycle and an alicyclic hydrocarbon ring.

Examples of the C6 to C20 aromatic ring structure include a benzene structure, a naphthalene structure, an anthracene structure, an indene structure, and a fluorene structure.

When R¹ is a monovalent cyclic organic group, R¹ may have a chain structure in addition to a cyclic structure. When R¹ is a group having both a chain structure and a cyclic structure, specific examples thereof include a group formed by bonding a cyclic structure to a divalent group obtained by removing one hydrogen atom from any of the aforementioned monovalent chain organic groups (preferably a monovalent linear-chain or branched saturated hydrocarbon group).

From the viewpoint of achieving suitable transparency of the resist film formed from the present composition, the monovalent organic group represented by R¹ preferably has no aromatic ring. More specifically, R¹ is preferably a substituted or unsubstituted monovalent chain hydrocarbon group, a monovalent group having an alicyclic hydrocarbon structure, or a monovalent group having an aliphatic heterocyclic structure, each group being bound to L¹ via a carbon atom. In addition, from the viewpoints of enhancing hydrophibicity of the resist film, and achieving a greater difference in solubility in a developer between the light-exposed part and the light-unexposed part, R¹ is more preferably a group having an alicyclic hydrocarbon structure or an aliphatic heterocyclic structure, still more preferably a group having a bridged alicyclic saturated hydrocarbon structure or a bridged aliphatic heterocyclic structure.

When R² is a substituted or unsubstituted divalent hydrocarbon group, examples of the divalent hydrocarbon group include a C1 to C10 divalent chain hydrocarbon group, a C3 to C20 divalent alicyclic hydrocarbon group, and a C6 to C20 divalent aromatic hydrocarbon group. Specific examples thereof include a group formed by removing one hydrogen atom from any of the monovalent hydrocarbon groups as exemplified in relation to R¹² in the aforementioned formula (3). Among them, the C1 to C20 divalent hydrocarbon group represented by R² is preferably a divalent chain hydrocarbon group or an alicyclic hydrocarbon group, preferably a C1 to C6 divalent chain hydrocarbon group or a C3 to C12 divalent alicyclic hydrocarbon group, more preferably a C1 to C6 linear-chain or branched alkanediyl group or a C3 to C8 cycloalkanediyl group.

When R² has a substituent, examples of the substituent include an iodine atom, a cyano group, and a C1 to C20 monovalent organic group. Examples of the C1 to C20 monovalent organic group include a C1 to C20 substituted or unsubstituted monovalent hydrocarbon group, and a monovalent group formed by substituting any methylene group of the hydrocarbon group by an ester group, an amide group, or a (thio)ether group. The monovalent hydrocarbon group may have a substituent such as a halogen atom or a hydroxy group.

When R¹ and R² are combined with each other and form, together with L¹ to which R¹ and R² are bound, a group having an aliphatic heterocyclic structure (hereinafter may also be referred to as a “group R^{M}”), examples of the aliphatic heterocyclic structure include a cyclic (thio) ether structure, a lactone structure, and a sultone structure. The aliphatic heterocyclic structure in group R^{M} may be directly bound to a sulfonate anion (—SO₃—) or bound to a sulfonate anion (—SO₃—) by the mediation of a divalent group (preferably a chain hydrocarbon group).

When L¹ is —CO—NR³—, examples of the monovalent hydrocarbon group represented by R³ include a C1 to C10 monovalent chain hydrocarbon group, a C3 to C10 monovalent alicyclic hydrocarbon group, and a C6 to C10 monovalent aromatic hydrocarbon group. Among them, the monovalent hydrocarbon group represented by R³ is preferably a C1 to C5 alkyl group, a C3 to C10 cycloalkyl group or a C6 to C10 aryl group.

When R² and R³ are combined with each other and form an aliphatic heterocyclic structure together with L¹(—CO—NR³—), examples of the aliphatic heterocyclic structure include a cyclic amide structure and a cyclic imide structure.

From the viewpoint of achieving suitable transparency of the resist film, R² is preferably, among others, a group having no aromatic ring. More specifically, preferably, R² is a substituted or unsubstituted divalent chain hydrocarbon group or a substituted or unsubstituted divalent alicyclic hydrocarbon group, or R² and R³ are combined with each other and form an aliphatic heterocyclic structure together with L¹.

Specific examples of the anion in the aforementioned formula (1) include the anions represented by the following formulas.

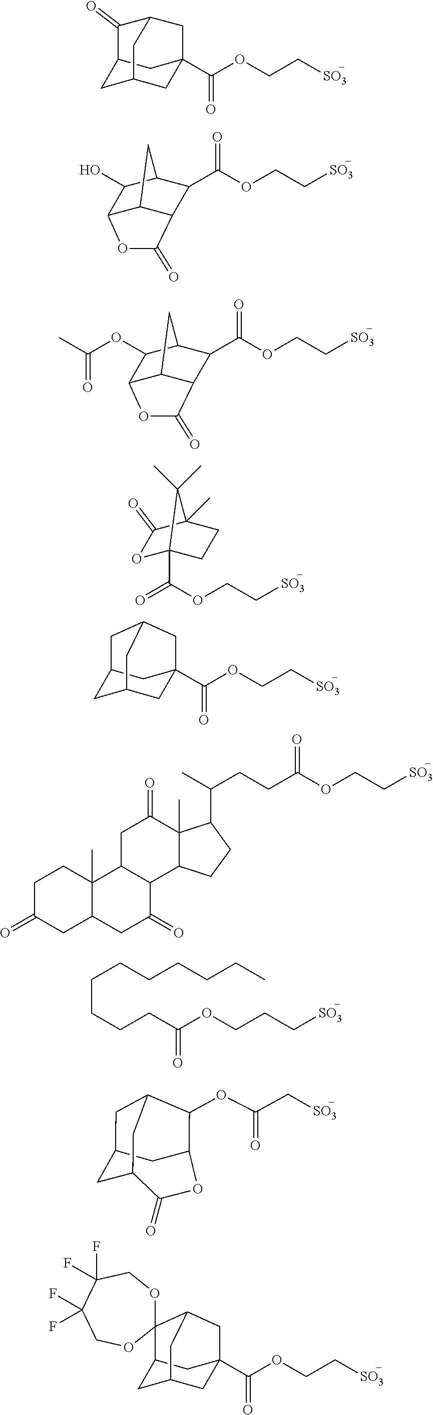

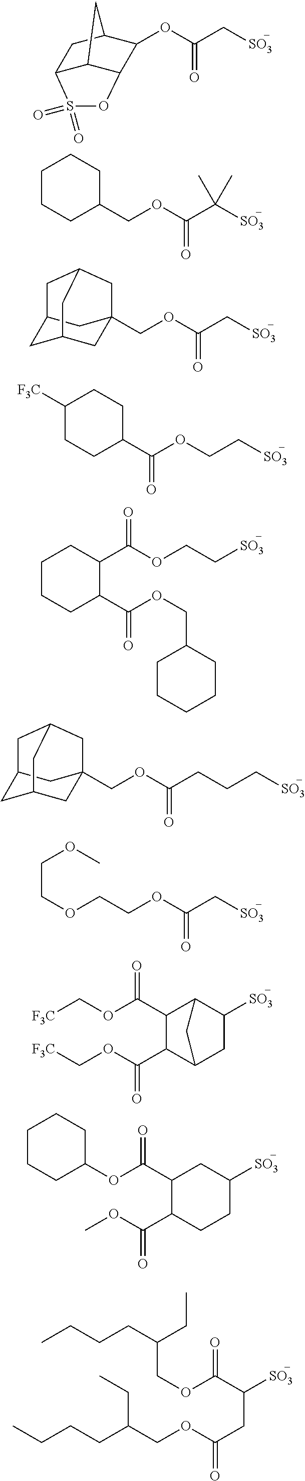

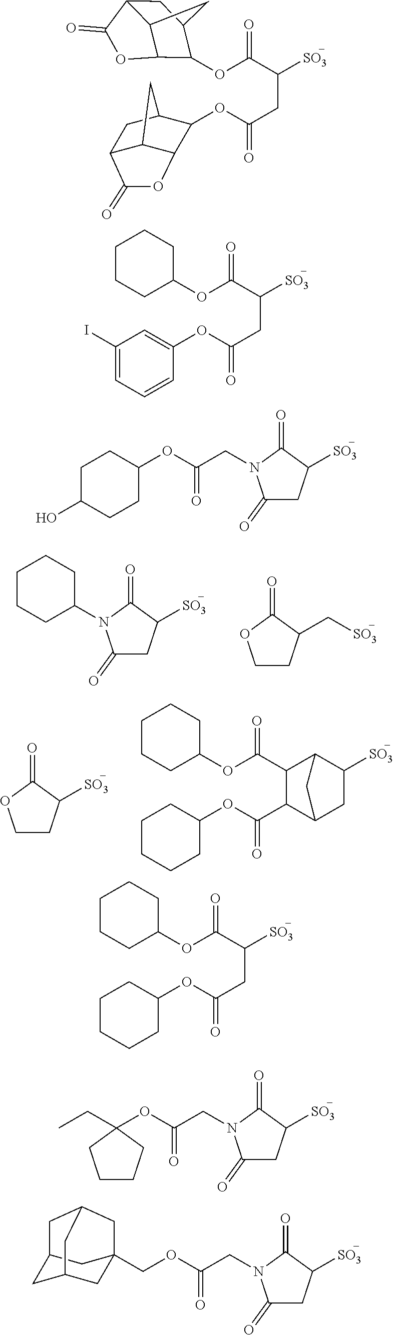

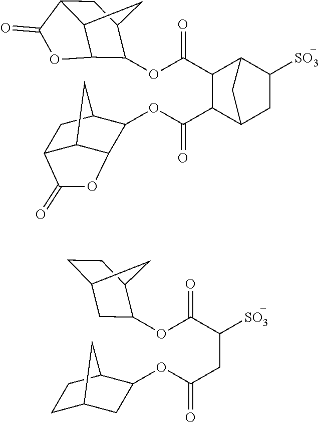

### Description of Cation

In the aforementioned formula (1), when R⁵ is a C1 to a C20 monovalent hydrocarbon group, examples of the monovalent hydrocarbon group include a C1 to C10 monovalent chain hydrocarbon group, a C3 to C20 monovalent alicyclic hydrocarbon group, and a C6 to C20 monovalent aromatic hydrocarbon group. Specific examples thereof include the same groups as exemplified in relation to R¹² in the aforementioned formula (3). The C1 to C20 monovalent hydrocarbon group represented by R⁵ is preferably, among others, a C1 to C8 monovalent hydrocarbon group, more preferably a C1 to C8 linear-chain or branched monovalent saturated hydrocarbon group, a C3 to C8 monovalent alicyclic hydrocarbon group, or a C6 to C8 monovalent aromatic hydrocarbon group.

Examples of the C1 to C20 monovalent halogenated hydrocarbon group represented by R⁵ include a group formed by substituting any hydrogen atom of the aforementioned C1 to C20 monovalent hydrocarbon group by a halogen atom. Examples of the halogen atom present in the C1 to C20 monovalent halogenated hydrocarbon group represented by R⁵ and the halogen atom represented by R⁵ include a fluorine atom, a chlorine atom, a bromine atom, and an iodine atom. The halogen atom is preferably a fluorine atom.

When two R⁵s are combined with each other and form, together with the carbon atom to which the R⁵s are bound, an alicyclic structure, the alicyclic structure forms a condensed ring with a benzene ring to which the two R⁵s are bound. Examples of the alicyclic structure include a cyclopentane ring and a cyclohexane ring.

Among the aforementioned examples, R⁵ is preferably a C1 to C8 alkyl group, a C1 to C8 halogenated alkyl group, a C3 to C8 cycloalkyl group, a C6 to C8 aryl group, or a halogen atom.

When R⁴ is a substituted or unsubstituted C1 to C20 monovalent hydrocarbon group, examples of the monovalent hydrocarbon group include a C1 to C10 monovalent chain hydrocarbon group, a C3 to C20 monovalent alicyclic hydrocarbon group, and a C6 to C20 monovalent aromatic hydrocarbon group. Specific examples thereof include the same groups as exemplified in relation to R¹² in the aforementioned formula (2). When R⁴ is a substituted C1 to C20 monovalent hydrocarbon group, examples of the substituent include a halogen atom, a hydroxy group, and a nitro group. Examples of the halogen atom represented by R⁴ include a fluorine atom, a chlorine atom, a bromine atom, and an iodine atom.

R⁴ is preferably, among others, a hydrogen atom, a C1 to C3 alkyl group, a halogen atom, a hydroxy group, or a nitro group, more preferably a hydrogen atom, a methyl group, or an ethyl group.

When L² is a divalent linking group, examples of the linking group include a C1 to C3 alkanediyl group, an ester group, and a (thio)ether group. From the viewpoint of enhancing sensitivity, L² is preferably, among others, a single bond.

Each of the parameters n1 and n2 is preferably 1 to 3, more preferably 2.

The parameter n3 is preferably 0 to 4, more preferably 0 to 3.

Specific examples of the cation in the aforementioned formula (1) include the cations represented by the following formulas.

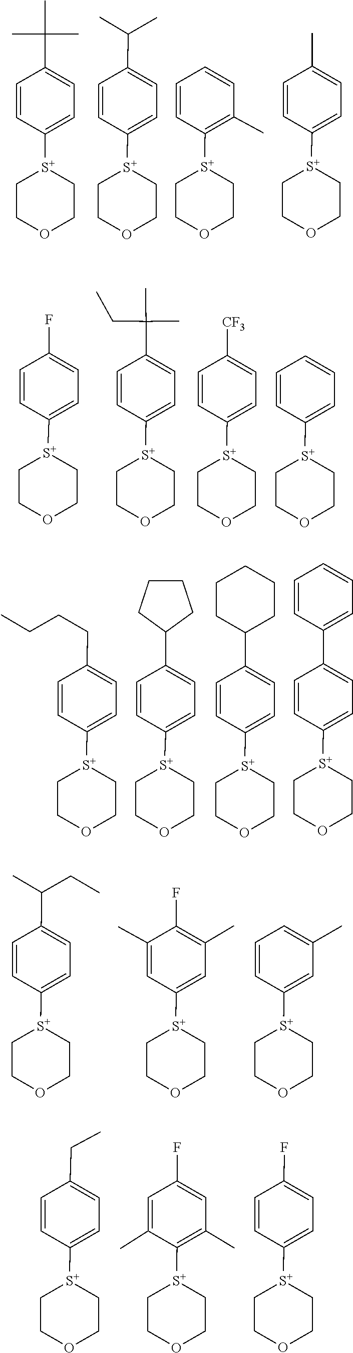

Specific examples of the compound (Q) include an onium salt compound formed from any one species of the anions mentioned as specific examples of the anion in the aforementioned formula (1), in combination with any one species of the cations mentioned as specific examples of the cation in the aforementioned formula (1).

The compound (Q) content of the present composition, with respect to 100 parts by mass of the polymer (A), is preferably 0.1 mass % or more, more preferably 0.5 mass % or more, still more preferably 1 mass % or more. Also, the compound (Q) content, with respect to 100 parts by mass of the polymer (A), is preferably 40 mass % or less, more preferably 30 mass % or less, still more preferably 20 mass % or less. By adjusting the compound (Q) content to satisfy the aforementioned conditions, the LWR performance, CDU performance, and pattern rectangularity of the present composition can be enhanced, whereby lithographic characteristics can be further enhanced. The compound (Q) may be used singly or in combination of two or more species.

### <Optional Component>

The present composition, containing the polymer (A) and the compound (Q), may further contains a component differing from the polymer (A) and the compound (Q) (i.e., an optional component). Examples of the optional component which may be incorporated into the present composition include a radiation-sensitive acid-generator, a solvent, and a high-fluorine content polymer.

### [Radiation-Sensitive Acid-Generator]

A radiation-sensitive acid-generator (hereinafter may also be referred simply as an “acid-generator”) is a substance which generates an acid upon exposure of the present composition to light. Typically, the acid-generator is a compound which evokes release of an acid-releasable group under the aforementioned “generally employed conditions,” to thereby generate an acid having an acidity higher than that of the acid generated by the compound (Q) (preferably, a strong acid such as sulfonic acid, imidic acid, or methide acid) in the composition (hereinafter may also be referred to as a “compound (B)”). In a preferred mode, both the polymer (A) and the compound (B) are incorporated into the present composition, and an acid-releasable group of the polymer (A) is released by the acid generated by the compound (B), to thereby generate an acid residue, whereby the dissolution rate of the polymer (A) in a developer is varied between the light-exposed part and the light-unexposed part.

The degree of acidity can be evaluated on the basis of acid dissociation constant (pKa). For example, the acid dissociation constant (pKa) of the acid generated by the light-degradable base is generally −3 or higher, preferably −1 to 7, more preferably 0 to 5.

No particular limitation is imposed on the compound (B) incorporated into the present composition, and a known radiation-sensitive acid-generator employed in resist pattern formation may be used. The compound (B) incorporated into the present composition is, for example, an onium salt formed of a radiation-sensitive onium cation and an organic anion. Among such compounds, the compound (B) is preferably any of the compounds represented by the following formula (2).

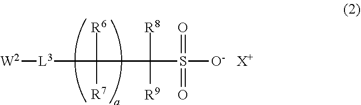

In the formula (2), W² represents a C3 to C40 monovalent organic group; L³ represents a single bond or a divalent bonding group; each of R⁶, R⁷, R³, and R⁹ independently represents a hydrogen atom, a C1 to C10 hydrocarbon group, a fluorine atom, or a C1 to C10 fluoroalkyl group; a is an integer of 0 to 8; when a is ≥2, a plurality of R⁶s and R⁷ s are identical to or different from one another; one or more members of (a×2+2) groups forming the group consisting of R⁶, R⁷, R³, and R⁹ in the formula are a fluorine atom or a fluoroalkyl group; and X⁺ represents a monovalent cation.

In the aforementioned formula (2), the C1 to C20 monovalent organic group represented by W² may be a group or a cyclic group. When W² is a monovalent chain organic group, specific examples thereof include a C1 to C20 linear-chain or branched saturated hydrocarbon group, a C2 to C20 linear-chain or branched unsaturated hydrocarbon group, a C1 to C20 monovalent group in which one or more hydrogen atoms of a chain hydrocarbon group are substituted by a halogen atom, a hydroxy group, a cyano group or the like, and a C2 to C20 monovalent group in which an ester group, a (thio)ether group, an amide group or the like is inserted into a carbon-carbon bond of a chain hydrocarbon group.

When W² is a monovalent cyclic organic group, no particular limitation is imposed on the cyclic organic group, so long as the group has a C3 to C20 cyclic structure. When W² is a monovalent cyclic organic group, examples of the cyclic structure included in W² include a C3 to C20 alicyclic hydrocarbon structure, a C3 to C20 aliphatic heterocyclic structure, and a C6 to C20 aromatic ring structure. These cyclic structures may have a substituent. Examples of the substituent include an alkoxy group, an alkoxycarbonyl group, a halogen atom (e.g., a fluorine atom, a chlorine atom, a bromine atom, or an iodine atom), a hydroxy group, and a cyano group. Also, when W² is a monovalent cyclic organic group, W² may include a chain structure in addition to the cyclic structure.

Examples of the C3 to C20 alicyclic hydrocarbon structure include a C3 to C20 alicyclic monocyclic structure and a C6 to C20 alicyclic polycyclic structure. The C3 to C20 alicyclic monocyclic structure and a C6 to C20 alicyclic polycyclic structure may be a saturated hydrocarbon structure or an unsaturated hydrocarbon structure. Also, the alicyclic polycyclic structure may be a bridged alicyclic hydrocarbon structure or a condensed alicyclic hydrocarbon structure.

Among the alicyclic monocyclic structures, examples of the saturated hydrocarbon structure include a cyclopentane structure, a cyclohexane structure, a cycloheptane structure, and a cyclooctane structure. Examples of the unsaturated hydrocarbon structure include a cyclopentene structure, a cyclohexene structure, a cycloheptene structure, a cyclooctene structure, and a cyclodecene structure. The alicyclic polycyclic structure is preferably a bridged alicyclic saturated hydrocarbon structure, and preferably includes a bicyclo[2.2.1]heptane structure, a bicyclo[2.2.2]octane structure, or a tricyclo[3.3.1.1^{3,7}]decane structure.

Examples of the C3 to C20 aliphatic heterocyclic structure include a cyclic ether structure, a lactone structure, a cyclic carbonate structure, a sultone structure, and a thioxane structure. The aliphatic heterocyclic structure may be a monocyclic structure or a polycyclic structure, and a bridged structure, a condensed ring structure, or a spiro ring structure. The C3 to C20 aliphatic heterocyclic structure represented by W² may be a combination of two or more of the bridged structure, the condensed ring structure, and the spiro ring structure. Examples of the C6 to C20 aromatic ring structure include a benzene structure, a naphthalene structure, an anthracene structure, an indene structure, and a fluorene structure.

From the viewpoint of achieving transparency of the resist film formed from the present composition and enhancing hydrophobicity of the film, to thereby gain a greater difference in solubility in a developer between the light-exposed part and the light-unexposed part, W² in the aforementioned formula (2) is preferably a monovalent cyclic organic group, more preferably has an alicyclic hydrocarbon structure or an aliphatic heterocyclic structure, still more preferably a bridged alicyclic saturated hydrocarbon structure or a bridged aliphatic heterocyclic structure. Also preferably, W² has no fluorine atom, from the viewpoint of sensitivity.

The divalent bonding group represented by L³ is preferably —O—, —CO—, —COO—, —O—CO—O—, —S—, —SO₂—, or —CONH—.

The C1 to C10 hydrocarbon group represented by any of R⁶, R⁷, R³, and R⁹ is preferably an alkyl group or a cycloalkyl group, particularly preferably an alkyl group. Among them, the hydrocarbon group represented by any of R⁶, R⁷, R³, and R⁹ is preferably a methyl group, an ethyl group, or an isopropyl group. Examples of the C1 to C10 fluoroalkyl group include a trifluoromethyl group, a 2,2,2-trifloroethyl group, a pentafluoroethyl group, a 2,2,3,3,3-pentafluoropropyl group, a 1,1,1,3,3,3-hexafluoropropyl group, a heptafluoro-n-propyl group, a heptafluoro-i-propyl group, a nonafluoro-n-butyl group, a nonafluoro-i-butyl group, a nonafluoro-t-butyl group, a 2,2,3,3,4,4,5,5-octafluoro-n-pentyl group, a tridecafluoro-n-hexyl group, and a 5,5,5-trifloro-1,1-diethylpentyl group. Of these, the fluoroalkyl group represented by any of R⁶, R⁷, R³, and R⁹ is preferably a C1 to C3 fluoroalkyl group, more preferably a trifluoromethyl group.

One or more members of (a×2+2) groups forming the group consisting of R⁶, R⁷, R³, and R⁹ in the formula are a fluorine atom or a fluoroalkyl group. For example, when a is 1, one or more members of R⁷, R⁸, R⁹, and R¹⁰ present in the formula are a fluorine atom, a fluoroalkyl group, or a fluorine atom or a fluoroalkyl group. When a is 2, one or more members of R⁷, R⁷, R⁸, R⁸, R⁹, and R¹⁰ present in the formula are a fluorine atom, a fluoroalkyl group, or a fluorine atom or a fluoroalkyl group. Of these, from the viewpoint of high acidity of the generated acid, the case in which R⁸, R⁹, or both are a fluorine atom or a trifluoromethyl group is preferred, with the case in which both R⁸ and R⁹ are a fluorine atom or a trifluoromethyl group being particularly preferred.

The parameter a is preferably 0 to 5, more preferably 0 to 2.

Specific examples of the anion included in the compound (B) include the anions represented by the following formula.

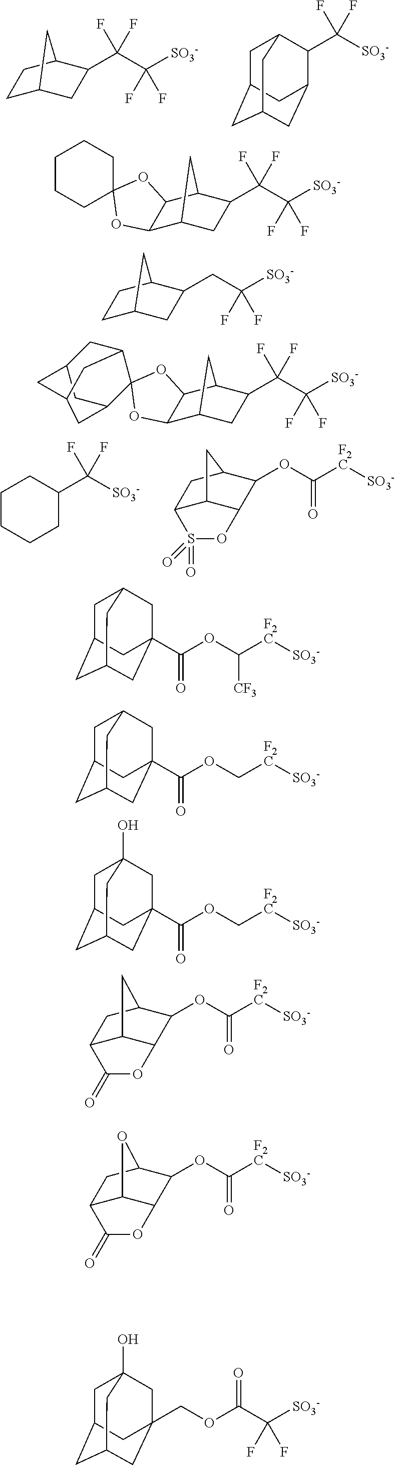

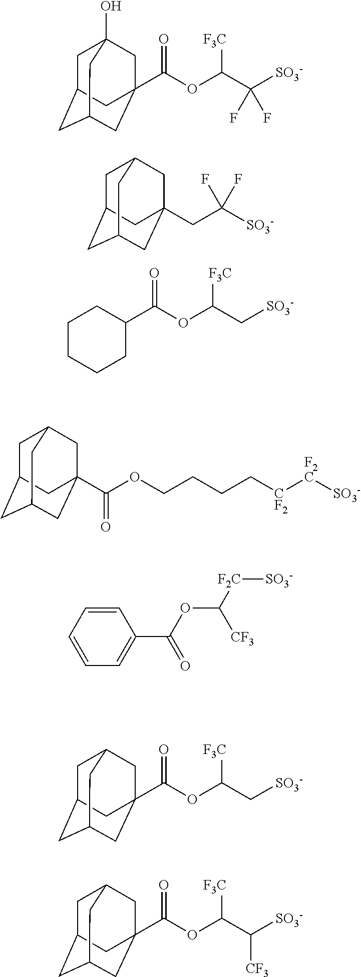

In the aforementioned formula (2), X⁺ represents a monovalent cation. The monovalent cation represented by X⁺ is preferably a monovalent radiation-sensitive onium cation, and examples thereof include radiation-degradable onium cations each containing an element such as S, I, O, N, P, C1, Br, F, As, Se, Sn, Sb, Te, or Bi. Specific examples of the radiation-degradable onium cations each having an element containing such an element include a sulfonium cation, a tetrahydrothiophenium cation, an iodonium cation, a phosphonium cation, a diazonium cation, and pyridinium cation. Of these, X⁺ is preferably a sulfonium cation or an iodonium cation. Specific examples include the cations represented by any of the aforementioned formulas (X-1) to (X-6).

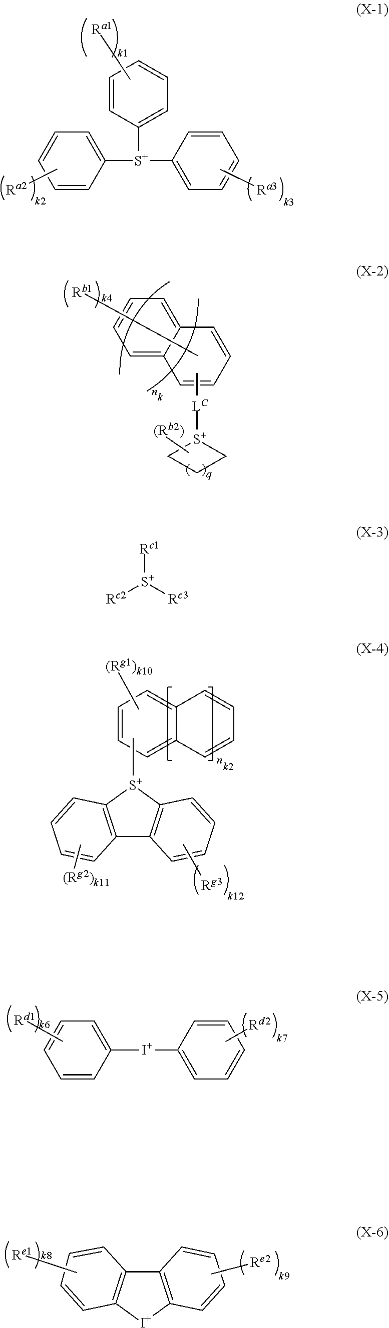

In the formula (X-1), each of R^{a1}, R^{a2}, and R^{a3} independently represents a substituted or unsubstituted C1 to C12 alkyl group, alkoxy group, alkylcarbonyloxy group, or cycloalkylcarbonyloxy group, a C3 to C12 monocyclic or polycyclic cycloalkyl group, a C6 to C12 monovalent aromatic hydrocarbon group, a hydroxy group, a halogen atom, —OSO₂—R^{P}, —SO₂—R^{Q}, or —S—R^{T}, or two or more of R^{a1}, R^{a2}, and R^{a} are combined to form a ring structure; the ring structure may include a hetero atom (e.g., an oxygen atom and a sulfur atom) between a carbon-carbon bond forming a skeleton; each of R^{P}, R^{Q}, and R^{T} independently represent a substituted or unsubstituted C1 to C12 alkyl group, a substituted or unsubstituted C5 to C25 monovalent alicyclic hydrocarbon group, or a substituted or unsubstituted C6 to C12 monovalent aromatic hydrocarbon group; each of k1, k2, and k3 is independently an integer of 0 to 5; when R^{a1} to R^{a3} and R^{P}, R^{Q}, and R^{T} respectively consist of a plurality of members, a plurality of R^{a1} to R^{a3} and R^{P}, R^{Q}, and R^{T} are identical to or different from one another; when each of R^{a1}, R^{a2}, and R^{a3} has a substituent, the substituent may be a hydroxy group, a halogen atom, a carboxy group, a protected hydroxy group, a protected carboxy group, —OSO₂—R^{P}, —SO₂—R^{Q}, or —S—R^{T}.

In the formula (X-2), R^{b1} represents a substituted or unsubstituted C1 to C20 alkyl group or alkoxy group, a substituted or unsubstituted C2 to C8 acyl group, a substituted or unsubstituted C6 to C8 monovalent aromatic hydrocarbon group, a halogen atom, or a hydroxy group; nₖ is 0 or 1; when nₖ is 0, k4 is an integer of 0 to 4; when nₖ is 1, k4 is an integer of 0 to 7; when a plurality of R^{b1}s are present, a plurality of R^{b1}s may be identical to or different from one another, or a plurality of R^{b1}s may be combined with one another to form a ring structure; R^{b2} represents a substituted or unsubstituted C1 to C7 alkyl group or a substituted or unsubstituted C6 or C7 monovalent aromatic hydrocarbon group; L^{C} represents a single bond or a divalent bonding group; k5 is an integer of 0 to 4; when a plurality of R^{b2}s are present, a plurality of R^{b2}s may be identical to or different from one another, or a plurality of R^{b2}s may be combined with one another to form a ring structure; q is an integer of 0 to 3; and the ring structure having S⁺ may include a hetero atom (e.g., an oxygen atom and a sulfur atom) between a carbon-carbon bond forming a skeleton.

In the formula (X-3), each of R^{c1}, R^{c2}, and R^{c3} independently represents a substituted or unsubstituted C1 to C12 alkyl group.

In the formula (X-4), R⁴ represents a substituted or unsubstituted C1 to C20 alkyl group or alkoxy group, a substituted or unsubstituted C2 to C8 acyl group, or a substituted or unsubstituted C6 to C8 aromatic hydrocarbon group, or a hydroxy group; nₖ₂ is 0 or 1; when nₖ₂ is 0, k10 is an integer of 0 to 4; when nₖ₂ is 1, k10 is an integer of 0 to 7; when a plurality of R^{g1} s are present, a plurality of R^{g1} s may be identical to or different from one another, or a plurality of R^{g1}s may be combined with one another to form a ring structure; each of R^{g2} and R^{g3} independently represents a substituted or unsubstituted C1 to C12 alkyl group, alkoxy group, or alkoxycarbonyloxy group, a substituted or unsubstituted C3 to C12 monocyclic or polycyclic cycloalkyl group, a substituted or unsubstituted C6 to C12 aromatic hydrocarbon group, a hydroxy group, or a halogen atom, or R^{g2} and R^{g3} are combined with each other to form a ring structure; each of k1l and k12 is independently an integer of 0 to 4; and when R^{g2} and R^{g3} respectively consist of a plurality of members, a plurality of R^{g2}s or R^{g3}s are identical to or different from one another.

In the formula (X-5), each of R^{d1} and R^{d2} independently represents a substituted or unsubstituted C1 to C12 alkyl group, alkoxy group, or alkoxycarbonyl group, a substituted or unsubstituted C6 to C12 aromatic hydrocarbon group, a halogen atom, a C1 to C4 halogenated alkyl group, or a nitro group, two or more of these groups are combined with one another to form a ring structure; each of k6 and k7 is independently an integer of 0 to 5; and when R^{d1} and R^{d2} respectively consist of a plurality of members, a plurality of R^{d}'s or R^{d2}s are identical to or different from one another.

In the formula (X-6), each of R^{e1} and R^{e2} independently represents a halogen atom, a substituted or unsubstituted C1 to C12 alkyl group, or a substituted or unsubstituted C6 to C12 aromatic hydrocarbon group; each of k8 and k9 is independently an integer of 0 to 4.

The cation represented by X⁺ in the aforementioned formula (2) is preferably, among others, the onium cations represented by the aforementioned formula (X-1), (X-2), or (X-5). Specific examples of the cation represented by X⁺ include the following structures represented by the formulas below.

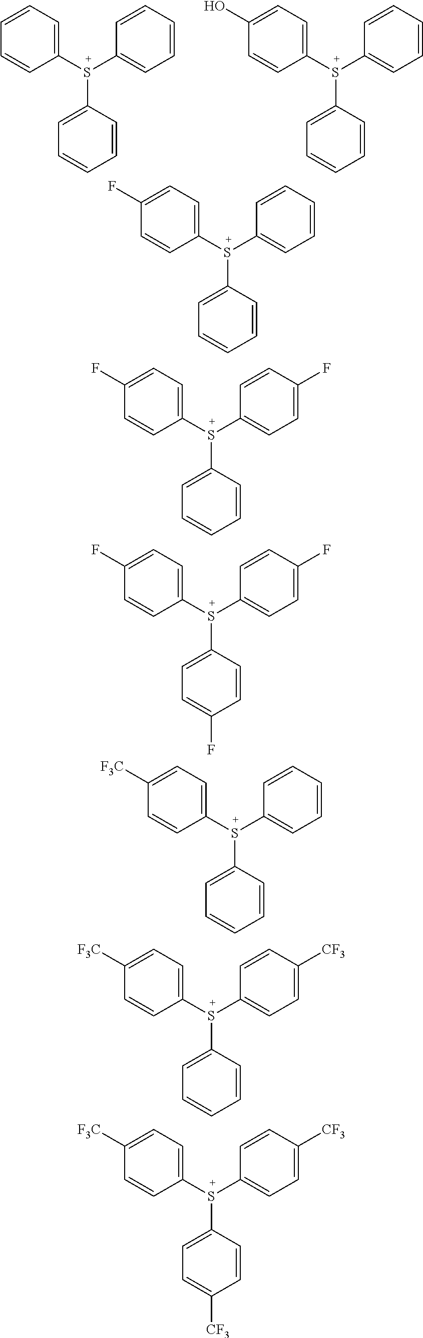

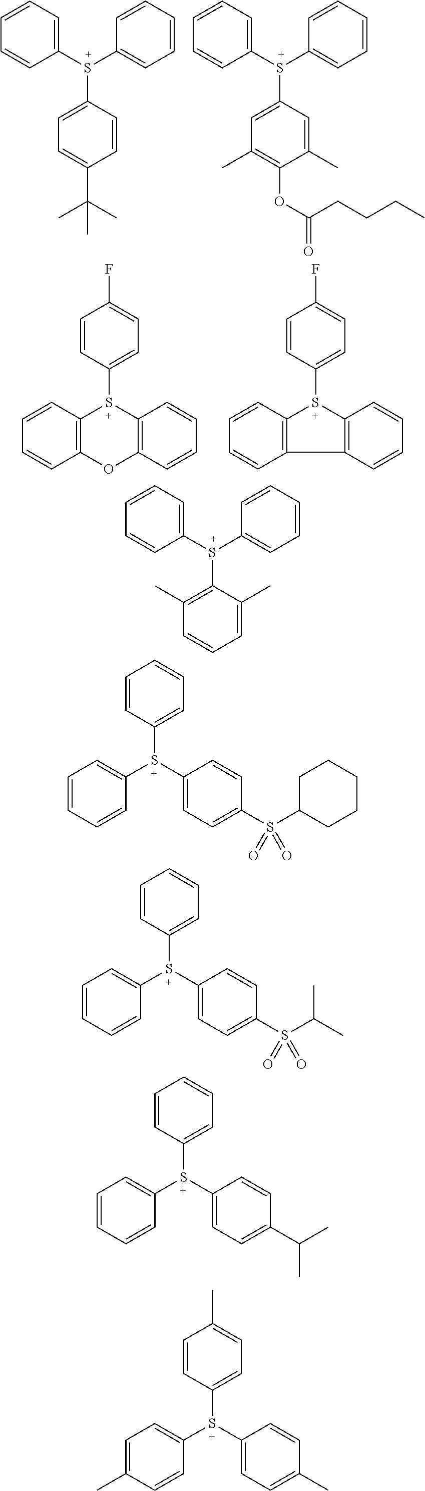

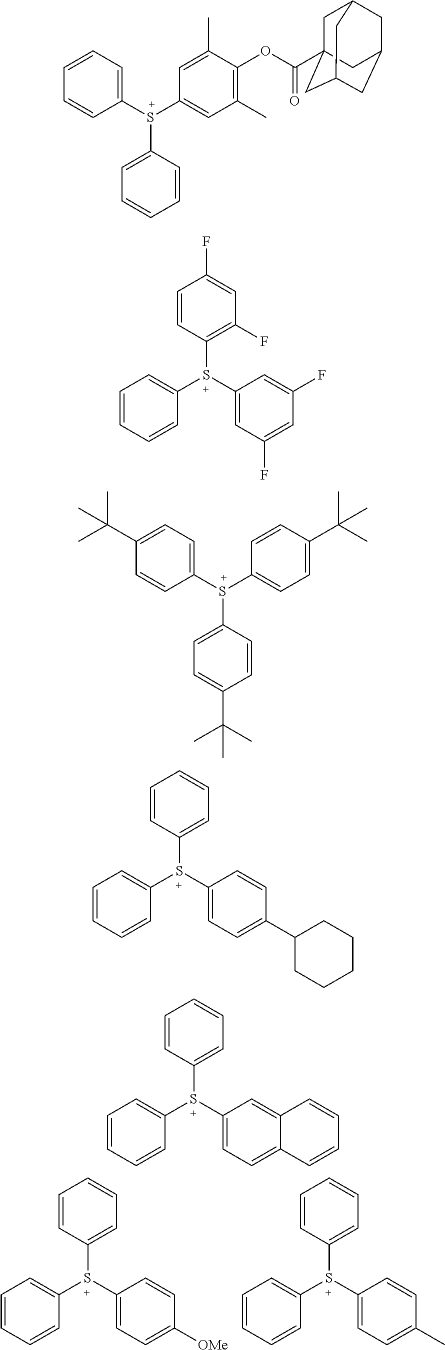

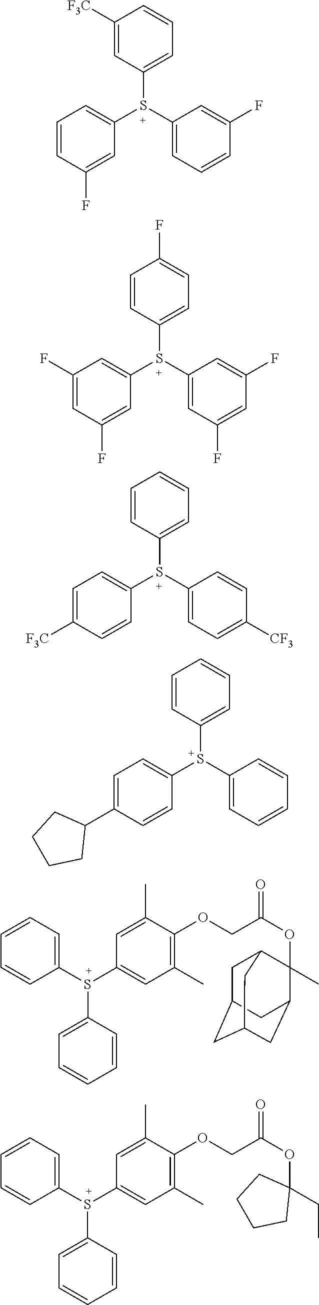

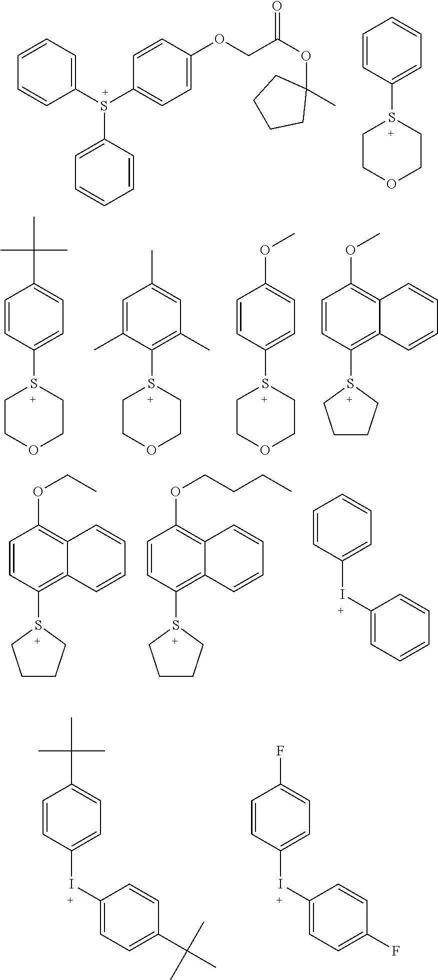

Specific examples of the compound represented by the aforementioned formula (2) include an onium salt compound formed from any one species of the anions mentioned as specific examples of the anion in the aforementioned formula (2), in combination with any one species of the cations mentioned as specific examples of the monovalent cation represented by X⁺. However, the compound represented by the aforementioned formula (2) is not limited to the above combinations. The compound represented by the aforementioned formula (2) may be used singly or in combination of two or more species.

In the present composition, the acid-generating agent content may be appropriately chosen in accordance with the type of the polymer (A) employed, light exposure conditions, target sensitivity, etc. The acid-generating agent content of the present composition, with respect to 100 parts by mass of the polymer (A), is preferably 1 part by mass or more, more preferably 2 parts by mass or more, still more preferably 5 parts by mass or more. Also, the acid-generating agent content of the present composition, with respect to 100 parts by mass of the polymer (A), is preferably 50 parts by mass or less, more preferably 40 parts by mass or less, still more preferably 30 parts by mass or less. By adjusting the acid-generating agent content to satisfy the aforementioned conditions, high sensitivity can be obtained during formation of a resist pattern, and suitable LWR performance, CDU performance, and pattern rectangularity can be attained.

### <Solvent>

No particular limitation is imposed on the solvent, so long as the solvent can dissolve or disperse the components incorporated into the present composition therein. Examples of the solvent include an alcohol, an ether, a ketone, an amide, an ester, and a hydrocarbon.

Examples of the alcohol include C1 to C18 aliphatic monoalcohols such as 4-methyl-2-pentanol and n-hexanol; C3 to C18 alicyclic monoalcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl isobutyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amide include cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the ester include monocarboxylic acid ester-type solvents such as n-butyl acetate and ethyl lactate; polyhydric alcohol carboxylates such as propylene glycol acetate; polyhydric alcohol partial ester carboxylates such as propylene glycol monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Among the above-exemplified solvents, the solvent preferably includes at least one member selected from the group consisting of the ester and the ketone, more preferably at least one member selected from the group consisting of polyhydric alcohol partial ether carboxylates and cyclic ketones, still more preferably one or more species of propylene glycol monomethyl ether acetate, ethyl lactate, and cyclohexanone. These solvents may be used singly or in combination of two or more species.

### <High-Fluorine Content Polymer>

The high-fluorine content polymer (hereinafter may also be referred to simply as a “polymer (E)”) is a polymer having a fluorine atom content (by mass) greater than that of the polymer (A). When the present composition contains the polymer (E), the polymer (E) may be localized to an upper layer of the resist film, with respect to the polymer (A). By virtue of the localization, water-repellency of the surface of the resist film during liquid immersion light exposure can be enhanced.

No particular limitation is imposed on the fluorine atom content of the polymer (E), so long as it is greater than the fluorine atom content of the polymer (A). The fluorine atom content of the polymer (E) is preferably 1 mass % or more, more preferably 2 mass % or more, still more preferably 4 mass % or more, particularly more preferably 7 mass % or more. Also, the fluorine atom content of the polymer [E] is preferably 60 mass % or less, more preferably 40 mass % or less, still more preferably 30 mass % or less. The fluorine atom content (mass %) of a polymer can be obtained by determining the structure of the polymer through ¹³C-NMR spectrometry or the like and calculating the content based on the structure determined.

Examples of the fluorine atom-containing structural unit incorporated into the polymer (E) (hereinafter may also be referred to as a “structural unit (F)”) include a structural unit (fa) and a structural unit (fb) specified below. The polymer (E) may include, as the structural unit (F), either a structural unit (fa) or a structural unit (fb), or both a structural unit (fa) and a structural unit (fb).

### [Structural Unit (Fa)]

The structural unit (fa) is a structural unit represented by the following formula (8-1). Through incorporation of the structural unit (fa), the fluorine atom content of the polymer (E) can be adjusted.

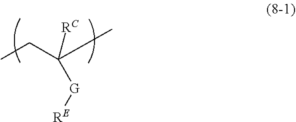

In formula (8-1), RC represents a hydrogen atom, a fluoro group, a methyl group, or a trifluoromethyl group; G represents a single bond, an oxygen atom, a sulfur atom, —COO—, —SO₂—O—NH—, —CONH—, or —O—CO—NH—; R^{E} represents a C1 to C20 monovalent fluorinated chain hydrocarbon group or a C3 to C20 monovalent fluorinated alicyclic hydrocarbon group.

In the aforementioned formula (8-1), from the viewpoint of co-polymerizability of a monomer forming the structural unit (fa), RC is preferably a hydrogen atom or a methyl group, more preferably a methyl group. Also, from the viewpoint of co-polymerizability of a monomer forming the structural unit (fa), G is preferably a single bond or —COO—, more preferably —COO—.

Examples of the C1 to C20 monovalent fluorinated chain hydrocarbon group represented by R^{E} include a C1 to C20 linear-chain or branched alkyl group in which the hydrogen atoms thereof is partially or completely substituted by a fluorine atom. Examples of the C3 to C20 monovalent fluorinated alicyclic hydrocarbon group represented by R^{E} include a C3 to C20 monocyclic or polycyclic alicyclic hydrocarbon group in which the hydrogen atoms thereof is partially or completely substituted by a fluorine atom. Of these, R^{E} is preferably a monovalent fluorinated chain hydrocarbon group, more preferably a monovalent fluorinated alkyl group, still more preferably a 2,2,2-trifloroan ethyl group, a 1,1,1,3,3,3-hexafluoropropyl group, or a 5,5,5-trifloro-1,1-diethylpentyl group.

When the polymer (E) includes the structural unit (fa), the relative amount of the structural unit (fa) in all the structural units forming the polymer (E) is preferably 30 mol % or more, more preferably 40 mol % or more, still more preferably 50 mol % or more. Also, the relative amount of the structural unit (fa) in all the structural units forming the polymer (E) is preferably 95 mol % or less, more preferably 90 mol % or less, still more preferably 85 mol % or less. By adjusting the structural unit (fa) content to satisfy the aforementioned conditions, the fluorine atom content (by mass) of the polymer (E) can be more appropriately tuned, whereby localization of the polymer (E) to the upper surface of the resist film can be further promoted. As a result, water-repellency of the surface of the resist film during liquid immersion light exposure can be further enhanced.

### [Structural Unit (Fb)]

The structural unit (fb) is a structural unit represented by the following formula (8-2). Through incorporation of the structural unit (fb), the solubility of the polymer (E) in an alkaline developer is enhanced, whereby generation of development failure can be further suppressed.

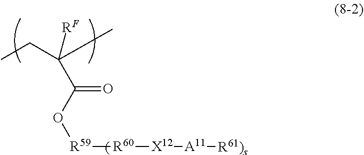

In formula (8-2), R^{F} represents a hydrogen atom, a fluoro group, a methyl group, or a trifluoromethyl group; R⁵⁹ represents a C1 to C20 (s+1)-valent hydrocarbon group or a group formed by bonding an oxygen atom, a sulfur atom, —NR′—, a carbonyl group, —CO—O—, or —CO—NH—to an end on the R⁶⁰ side of the hydrocarbon group; R′ represents a hydrogen atom or a monovalent organic group; R⁶⁰ represents a single bond or a C1 to C20 divalent organic group; X¹² represents a single bond, a C1 to C20 divalent hydrocarbon group, or a C1 to C20 divalent fluorinated chain hydrocarbon group; A¹¹ represents an oxygen atom, —NR″—, —CO—O—*, or —SO₂—O—*; R″ represents a hydrogen atom or a C1 to C10 monovalent hydrocarbon group; “*” represents a connection site to R⁶¹; R⁶¹ represents a hydrogen atom or a C1 to C30 monovalent organic group; s is an integer of 1 to 3; and when s is 2 or 3, a plurality of R⁶⁰, X¹², A¹¹, and R⁶¹ are identical to or different from one another.

In one case, the structural unit (fb) has an alkali-soluble group, and in the other case, the structural unit (fb) has a group which is released by the action of alkali, to thereby enhance solubility in an alkaline developer (hereinafter may also be referred to simply as an “alkali-releasable group”).

In the case where the structural unit (fb) has an alkali-soluble group, R⁶¹ represents a hydrogen atom, and A¹¹ represents an oxygen atom, —COO—*, or —SO₂O—*. “*” represents a connection site to R⁶¹. X¹² represents a single bond, a C1 to C20 divalent hydrocarbon group, or a C1 to C20 divalent fluorinated chain hydrocarbon group. When A¹¹ is an oxygen atom, X¹² is a fluorinated hydrocarbon group having a fluorine atom or a fluoroalkyl group on the carbon atom bound to A¹¹. R⁶⁰ represents a single bond or a C1 to C20 divalent organic group. When s is 2 or 3, a plurality of R⁶⁰, X¹², A¹¹, and R⁶¹ are identical to or different from one another. By virtue of the presence of an alkali-soluble group in the structural unit (fb), affinity to an alkaline developer can be enhanced, to thereby suppress development failure.

When the structural unit (fb) has an alkali-releasable group, R⁶¹ represents a C1 to C30 monovalent organic group, and A¹¹ represents an oxygen atom, —NR″—, —COO—*, or —SO₂O—*. “*” represents a connection site to R⁶¹. X¹² represents a single bond or a C1 to C20 divalent fluorinated chain hydrocarbon group. R⁶⁰ represents a single bond or a C1 to C20 divalent organic group. When A¹¹ is —COO—*or —SO₂O—*, X¹² or R⁶¹ has a fluorine atom on the carbon atom bound to A¹¹ or a carbon atom adjacent to the carbon atom bound to A¹¹. When A¹¹ is an oxygen atom, X¹² or R⁶⁰ is a single bond ζ, and R⁵⁹ is a structure in which a carbonyl group is bound to an end on the R⁶⁰ side of the C1 to C20 hydrocarbon group. R⁶¹ represents an organic group having a fluorine atom. When s is 2 or 3, a plurality of R⁶⁰, X¹², A¹¹, and R⁶¹ are identical to or different from one another. By virtue of the presence of an alkali-soluble group in the structural unit (fb), a hydrophobic surface of the resist film changes to a hydrophilic surface in the alkali in an alkali development step. As a result, affinity to a developer can be enhanced, and development failure can be more efficiently suppressed. An example of the structural unit (fb) having an alkali-releasable group in which A¹¹ is —COO—*, and R⁶¹ or X¹² or both have a fluorine atom is particularly preferred.

When the polymer (E) includes the structural unit (fb), the relative amount of the structural unit (fb) in all the structural units forming the polymer (E) is preferably 40 mol % or more, more preferably 50 mol % or more, still more preferably 60 mol % or more. Also, the relative amount of the structural unit (fb) in all the structural units forming the polymer (E) is preferably 95 mol % or less, more preferably 90 mol % or less, still more preferably 85 mol % or less. By adjusting the structural unit (fb) content to satisfy the aforementioned conditions, water-repellency of the surface of the resist film during liquid immersion light exposure can be enhanced.

In addition to the structural units (fa) and (fb), the polymer (E) may further include a structural unit (I) having an acid-releasable group, or a structural unit having an alicyclic hydrocarbon structure represented by the following formula (9) (hereinafter may also be referred to as a “structural unit (G)”).

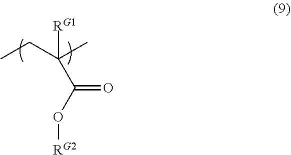

In the formula (9), R^{G1} represents a hydrogen atom, a fluorine atom, a methyl group, or a trifluoromethyl group; and R^{G2} represents a C3 to C20 monovalent alicyclic hydrocarbon group.

In the aforementioned formula (9), examples of the C3 to C20 monovalent alicyclic hydrocarbon group represented by R^{G2} include the hydrocarbon groups as exemplified in relation to the C3 to C20 monovalent alicyclic hydrocarbon group represented by any of R¹³ to R¹⁵ in the aforementioned formula (3).

When the polymer (E) includes the structural unit represented by the aforementioned formula (9), the relative amount of the structural unit in all the structural units forming the polymer (E) is preferably 10 mol % or more, more preferably 20 mol % or more, still more preferably 30 mol % or more. Also, the relative amount of the structural unit represented by the aforementioned formula (9) in all the structural units forming the polymer (E) is preferably 70 mol % or less, more preferably 60 mol % or less, still more preferably 50 mol % or less.

The Mw of the polymer (E), which is determined through GPC, is preferably 1,000 or more, more preferably 3,000 or more, still more preferably 4,000 or more. Also, the Mw of the polymer (E) is preferably 50,000 or less, more preferably 30,000 or less, still more preferably 20,000 or less. The molecular weight distribution (Mw/Mn), which is the ratio of Mw to Mn of the polymer (E) determined through GPC, is preferably 1 to 5, more preferably 1 to 3.

When the present composition contains the polymer (E), the relative amount of the polymer (E) in the present composition, with respect to 100 parts by mass of the polymer (A), is preferably 0.1 parts by mass or more, more preferably 0.5 parts by mass or more, still more preferably 1 part by mass or more. Also, the polymer (E) content, with respect to 100 parts by mass of the polymer (A), is preferably 10 parts by mass or less, more preferably 7 parts by mass or less, still more preferably 5 parts by mass or less. The present composition may contain the polymer (E) singly or in combination of two or more species.

### <Additional and Optional Component>

The present composition may further contain a component which differs from the aforementioned polymer (A), compound (Q), compound (B), solvent, and polymer (E) (hereinafter the component may also be referred to as “additional and optional component”). Examples of the additional and optional component include an acid diffusion control agent other than the compound (Q) (e.g., a nitrogen-containing compound represented by “N (R^{N1}) (R^{N2}) (R^{N3})” (wherein each of R^{N1}, R^{N2}, and R^{N3} independently represents a hydrogen atom, a substituted or unsubstituted alkyl group, a substituted or unsubstituted cycloalkyl group, a substituted or unsubstituted aryl group, or an substituted or unsubstituted aralkyl group), or a light-degradable base differing from a compound represented by the aforementioned formula (1)), a surfactant, a compound having an alicyclic skeleton (e.g., 1-adamantanecarboxylic acid, 2-adamantanone, or t-butyl deoxycholate), a sensitizer, and a localization accelerator. So long as the effect of the present disclosure are not impaired, the additional and optional component content of the present composition may be appropriately set depending on the property of the component.

When the acid diffusion control agent other than the compound (Q) is incorporated into the present composition, from the viewpoint of yielding a radiation-sensitive composition exhibiting favorable sensitivity and excellent CDU performance and pattern rectangularity, the relative amount of the acid diffusion control agent other than the compound (Q), with respect to the entire amount of the acid diffusion control agents contained in the present composition, is preferably 60 mass % or less, more preferably 50 mass % or less.

### <Method of Producing Radiation-Sensitive Composition>

The present composition may be produced through, for example, the following procedure: mixing the polymer (A) and the compound (Q) with optional components such as the solvent at a desired ratio and filtering the resultant mixture preferably by means of a filter (e.g., a filter having a pore size of about 0.2 μm) or the like. The solid content of the present composition is preferably 0.1 mass % or more, more preferably 0.5 mass % or more, still more preferably 1 mass % or more. Also, the solid content of the present composition is preferably 50 mass % or less, more preferably 20 mass % or less, still more preferably 5 mass % or less. By adjusting the solid content of the present composition to satisfy the above conditions, coatability of the composition can be enhanced, to thereby obtain a resist pattern having a suitable shape.

The thus-obtained present composition may also be used as a composition for forming a positive pattern, which is employed for pattern formation by use of an alkaline developer. Alternatively, the present composition may be used as a composition for forming a negative pattern, which is employed for pattern formation by use of a developer containing organic solvent. Among them, the present composition is particularly suited for a negative-type pattern forming composition employed with an organic solvent developer, from the viewpoint of a high effect of providing excellent pattern rectangularity through development of a light-exposed resist film, while high sensitivity is secured.

### <Method of Forming Resist Pattern>

The resist pattern formation method of the present disclosure includes a step of applying the present composition on one surface of a substrate (hereinafter may also be referred to as a “application step”), a step of exposing a resist film obtained in the application step to a radiation (hereinafter may also be referred to as a “exposure step”), and a step of developing the radiation-exposed resist film (hereinafter may also be referred to as a “development step”). Examples of the pattern obtained through the resist pattern formation method of the present disclosure include a line-and-space pattern and a hole pattern. Since a resist film is formed by use of the present composition in the resist pattern formation method of the present disclosure, suitable sensitivity and lithographic characteristics are achieved, and a resist pattern which has few development failure can be formed. The steps will next be described in detail.

### [Application Step]

In the application step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum.

Alternatively, an organic or inorganic anti-reflection film (see, for example, Japanese Patent Publication (kokoku) No. 1994-12452 or Japanese Patent Application laid-Open (kokai) No. 1984-93448) may be formed on a substrate to be used. Examples of the method of applying the present composition include spin coating, flow casting, and roller coating. After application, the applied composition may be subjected to pre-baking (PB) so as to evaporate the solvent remaining in the coating film. The temperature of PB is preferably 60° C. or higher, more preferably 80° C. or higher, and preferably 140° C. or lower, more preferably 120° C. or lower. The time of PB is preferably 5 seconds or longer, more preferably 10 seconds or longer, and preferably 600 seconds or shorter, more preferably 300 seconds or shorter. The average thickness of the formed resist film is preferably 10 to 1,000 nm, more preferably 20 to 500 nm.

Particularly, since the present composition allows to form a resist film having high transparency with high sensitivity, when a resist film is exposed to light to form a pattern, the radiation can sufficiently reach a deep part of the film. The present composition having such a property exhibits excellent lithographic characteristics (e.g., LWR performance and CDU performance), also when the composition is used for forming a thick resist film. Thus, the present composition is particularly suited for forming a thick resist film. In formation of a thick resist film, the average thickness of the resist film is preferably 50 nm or more, more preferably 70 nm or more. Also, the average thickness of the resist film is, for example, 1,000 nm or less, preferably 500 nm or less.

In the case where liquid immersion light exposure is conducted in the subsequent exposure step, in order to avoid direct contact of the immersion liquid with the resist film, a protective film which is undissolved in the immersion liquid is further provided on the resist film formed from the present composition, regardless of the presence of a water-repellent polymer additive such as the polymer (E) in the present composition. As the protective film for liquid immersion, there may be used any of a solvent-peelable protective film which can be removed with a solvent before the development step (see, for example, Japanese Patent Application laid-Open (kokai) No. 2006-227632) and a developer-peelable protective film which is removed simultaneously with conducting the development step (see, for example, WO 2005/069076 or 2006/035790). From the viewpoint of through-put, a developer-peelable protective film for liquid immersion is preferably used.

### [Exposure Step]

In the exposure step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a UV ray, a far-UV ray, an extreme UV (EUV) ray, an X-ray, and a γ-ray; and charged particle rays such as an electron beam and an α-ray. Among them, the radiation applied to the resist film formed from the present composition is preferably a far-UV ray, an EUV ray, or an electron beam, more preferably ArF excimer laser light (wavelength: 193 nm), KrF excimer laser light (wavelength: 248 nm), an EUV ray, or an electron beam, still more preferably ArF excimer laser light, an EUV ray, or an electron beam.

After completion of the above light exposure, post exposure baking (PEB) is preferably performed, so as to promote dissociation of an acid-releasable group by the mediation of an acid generated through light exposure by an acid-generating agent in the light-exposed part of the resist film. Through PEB, the difference in dissolution performance with respect to a developer between the exposed part and the unexposed part can be increased. The temperature of PEB is preferably 50° C. or higher, more preferably 80° C. or higher, and preferably 180° C. or lower, more preferably 130° C. or lower. The time of PEB is preferably 5 seconds or longer, more preferably 10 seconds or longer, and preferably 600 seconds or shorter, more preferably 300 seconds or shorter.

### [Development Step]

In the development step, the resist film which has been exposed to light in the above step is developed, whereby a resist pattern of interest can be formed. The developer may be an alkaline developer or an organic solvent developer. The developer may be appropriately chosen in accordance with the target type of the pattern (i.e., a positive-type pattern or a negative-type pattern).

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, 1,5-diazabicyclo[4.3.0]-5-nonene, and the like is dissolved. Among such alkaline solutions, an aqueous TMAH solution is preferred, with 2.38-mass % aqueous TMAH solution being more preferred.

In the case of development with an organic solvent, examples of the developer include of organic solvents such as hydrocarbons, ethers, esters, ketones, and alcohols; and a solvent containing any of the above organic solvents. Examples of the organic solvent include one or more solvents as exemplified in relation to the solvent which may be added to the present composition. Among them, esters, esters, and ketones are preferred. Among ethers, a glycol ether is preferred, with ethylene glycol monomethyl ether and propylene glycol monomethyl ether being more preferred. Among the esters, acetate esters are preferred, with n-butyl acetate and amyl acetate being more preferred. Among the ketones, chain ketones are preferred, with 2-heptanone being more preferred. The organic solvent content of the developer is preferably 80 mass % or more, more preferably 90 mass % or more, still more preferably 95 mass % or more, particularly preferably 99 mass % or more. Examples of developer components other than organic solvent include water and silicone oil.

Examples of the development method include a dipping method (i.e., dipping a substrate in a bath filled with a developer for a specific time); a paddle method (i.e., putting a developer in a substrate to form a drop by surface tension and allowing the substrate to stand for a specific time); a spray method (i.e., spraying a developer onto a substrate); and a dynamic dispense method (i.e., continuously jetting a developer at a specific speed to a substrate rotating at a specific speed through a developer jetting nozzle with scanning). After development, washing with a rinsing liquid such as water or alcohol, and drying are generally conducted.

The present composition described hereinabove, containing the polymer (A) and the compound (Q), exhibits high sensitivity in formation of a resist pattern and provides excellent LWR performance, CDU performance, and pattern rectangularity. Thus, the present composition can be suitably employed in a semiconductor device processing or the like, where a further process shrinkage will proceed in future.

According to the present disclosure described in detail hereinabove, the following means are provided.

[Means 1] A radiation-sensitive composition containing a polymer having an acid-releasable group and a compound represented by the aforementioned formula (1).
[Means 2] The radiation-sensitive composition as described in [Means 1], wherein R² represents a substituted or unsubstituted divalent chain hydrocarbon group or a substituted or unsubstituted divalent alicyclic hydrocarbon group, or an aliphatic heterocyclic structure formed by combining R² and R³ with each other, together with L¹.
[Means 3] The radiation-sensitive composition as described in [Means 1] or [Means 2], wherein R¹ represents a substituted or unsubstituted monovalent chain hydrocarbon group, a monovalent group having an alicyclic hydrocarbon structure, or a monovalent group having an aliphatic heterocyclic structure, each group being bound to L¹ via a carbon atom.
[Means 4] The radiation-sensitive composition as described in any of [Means 1] to [Means 3], wherein L² is a single bond.
[Means 5] A radiation-sensitive composition as described in any of [Means 1] to [Means 4], wherein the composition contains a compound (B) which generates, in the composition, an acid having an acidity higher than the compound (Q) through irradiation with a radiation.
[Means 6] The radiation-sensitive composition as described in [Means 5], wherein the compound (B) is a compound represented by the aforementioned formula (2).
[Means 7] A pattern formation method, including forming a resist film by applying the radiation-sensitive composition as recited in any of [Means 1] to [Means 6] onto a substrate, exposing the resist film to a radiation, and developing the radiation-exposed resist film.
[Means 8] The pattern formation method as described in [Means 7], wherein in the developing, the radiation-exposed resist film is developed with an organic solvent developer.
[Means 9] A light-degradable base represented by the aforementioned formula (1).

### EXAMPLES

The present disclosure will next be described in detail by way of examples, which should not be construed as limiting the disclosure thereto. Unless otherwise specified, the units “part(s)” and “%” in the Examples are on a mass basis. Measurements in the Examples and Comparative Examples were conducted through the following procedures.

### [Weight Average Molecular Weight (Mw), Number Average Molecular Weight (Mn), and Molecular Weight Distribution (Mw/Mn)]

The Mw and Mn of a polymer were determined through gel permeation chromatography (GPC) with GPC columns (G2000HXL×2, G3000HXL×1, and G4000HXL×1) (products of Tosoh Corp.) under the following conditions; i.e., flow rate: 1.0 mL/min, eluent: tetrahydrofuran, sample concentration: 1.0 mass %, sample injection: 100 μL, column temperature: 40° C., detector: differential refractometer, and standard: monodispersed polystyrene. The molecular weight distribution (Mw/Mn) was calculated from the measurements of Mw and Mn.

### [¹³C-NMR Analysis]

¹³C-NMR analysis of a polymer was performed by means of a nuclear magnetic resonance apparatus (“JNM-Delta400,” product of JEOL).

In preparation of the radiation-sensitive resin compositions, the following resin [A], radiation-sensitive acid-generating agent [B], acid diffusion control agent [C], solvent [D], and high-fluorine content resin [E] were used.

### <Resin [A] and High-Fluorine Content Resin [E]>

### Synthesis of Resin [A] and High-Fluorine Content Resin [E]

Monomers used in synthesis of the resin and high-fluorine content resins are as follows. In the following Synthesis Examples, unless otherwise specified, the unit “parts by mass” is based on the total mass of the monomers used as 100 parts by mass. The unit “mol %” shown in parenthesis is based on the total amount by mole of the monomers used as 100 mol %.

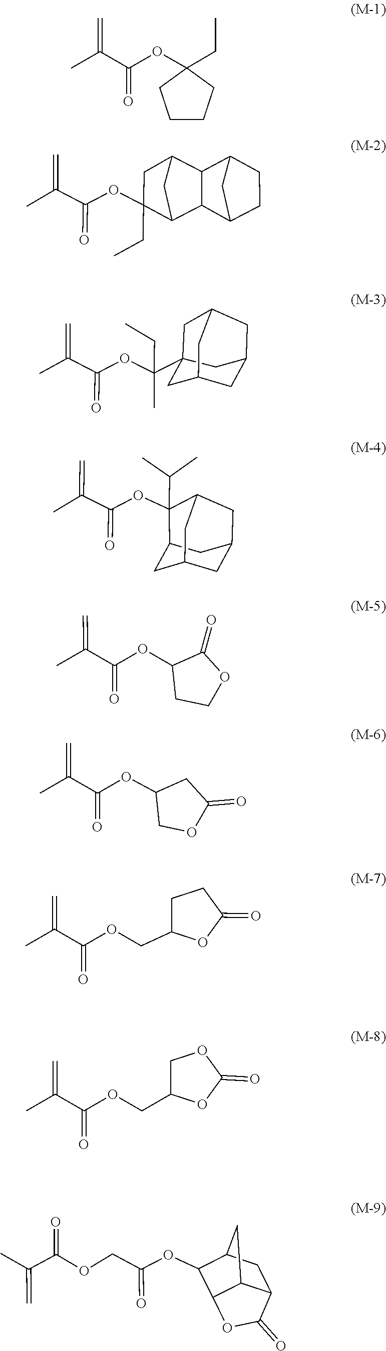

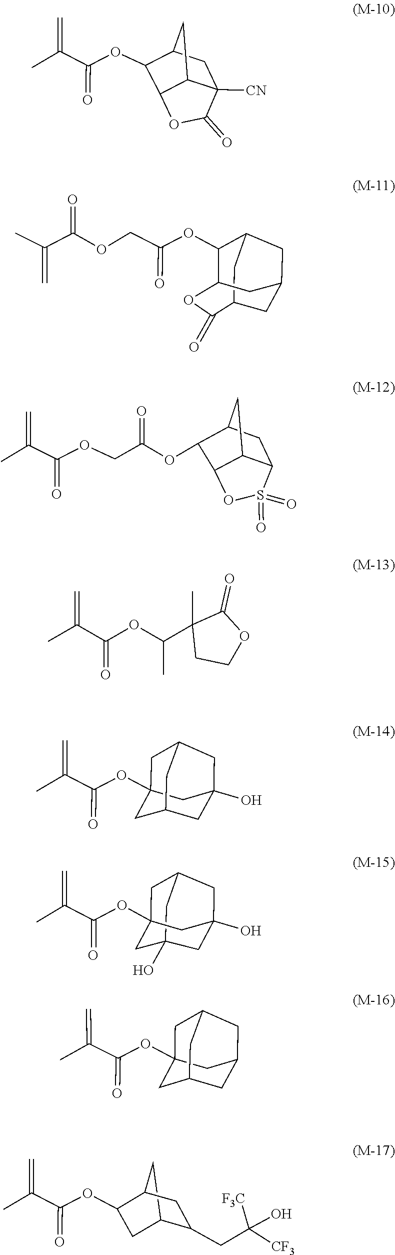

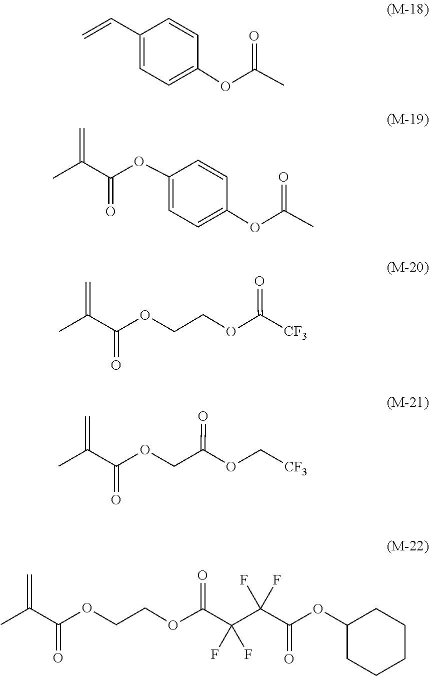

### Synthesis Example 11

### (Synthesis of Resin (A-1))

Monomer (M-1), monomer (M-4), monomer (M-5), monomer (M-11), and monomer (M-14) were dissolved in 2-butanone (200 parts by mass) so that the proportions by mole thereof were adjusted to 40/10/20/20/10 (mol %). To the solution, azobisisobutyronitrile (AIBN) (3 mol % with respect to the total amount of the monomers used as 100 mol %) serving as an initiator was added, to thereby prepare a monomer solution. Separately, 2-butanone (100 parts by mass) was added to a reaction container. The atmosphere of the container was purged with nitrogen for 30 minutes. Subsequently, the inside temperature of the reaction container was adjusted to 80° C., and the above-prepared monomer solution was added dropwise to the container over 3 hours under stirring. Polymerization reaction was conducted for 6 hours, wherein the time of start of dropwise addition was employed as the time of initiating polymerization reaction. After completion of polymerization reaction, the reaction mixture was water-cooled to 30° C. or lower. The cooled polymer solution was transferred to methanol (2,000 parts by mass), and the precipitated white powder was separated through filtration. The thus-separated white powder was washed twice with methanol, and separated through filtration, followed by drying at 50° C. for 24 hours, to thereby yield resin (A-1) in the form of white powder (yield: 80%). The resin (A-1) was found to have an Mw of 9,100 and an Mw/Mn of 1.54. Through ¹³C-NMR analysis, the proportions of structural units derived from monomer (M-1), monomer (M-4), monomer (M-5), monomer (M-11), and monomer (M-14) were found to be 40.6 mol %, 9.7 mol %, 21.1 mol %, 20.5 mol %, and 8.1 mol %.

### Synthesis Examples 2 to 11

### (Synthesis of Resins (A-2) to (A-11)

The procedure of Synthesis Example 1 was repeated, except that the types and amounts of the monomers were changed as shown in Table 1 were employed, to thereby yield resins (A-2) to (A-11), respectively. Table 1 also shows structural unit proportions (mol %) and physical properties (Mw and Mw/Mn) of each of the produced resins. Notably, the symbol “-” in Table 1 refers to “no use of the relevant monomer” (this applied to the Tables hereinafter).

TABLE 1

| | | Synthesis | | Synthesis | | Synthesis | | Synthesis | |
|---|---|---|---|---|---|---|---|---|---|
| | | Example 1 | | Example 2 | | Example 3 | | Example 4 | |
| Resin [A] | | A-1 | | A-2 | | A-3 | | A-4 | |
| Monomer | Type | M-1 | M-4 | M-1 | M-2 | M-1 | M-3 | M-1 | M-3 |
| providing | Amount of | 40 | 10 | 30 | 10 | 30 | 10 | 35 | 20 |
| structural unit | monomer | | | | | | | | |
| (I) | (mol %) | | | | | | | | |
| | Proportion | 40.6 | 9.7 | 31.4 | 8.0 | 31.9 | 8.9 | 34.8 | 18.8 |
| | of | | | | | | | | |
| | structural | | | | | | | | |
| | unit | | | | | | | | |
| | (mol %) | | | | | | | | |

| Monomer | Type | M-5 | M-11 | M-6 | | M-5 | M-12 |
|---|---|---|---|---|---|---|---|
| providing | Amount of | 20 | 20 | 60 | | 60 | 45 |
| structural unit | monomer | | | | | | |
| (II-1) | (mol %) | | | | | | |
| | Proportion | 21.1 | 20.5 | 60.6 | | 59.2 | 46.4 |
| | of | | | | | | |
| | structural | | | | | | |
| | unit | | | | | | |
| | (mol %) | | | | | | |
| Monomer | Type | M-14 | | — | | — | — |
| providing | Amount of | 10 | | — | | — | — |
| structural unit | monomer | | | | | | |
| (II-2) | (mol %) | | | | | | |
| | Proportion | 8.1 | | — | | — | — |
| | of | | | | | | |
| | structural | | | | | | |
| | unit | | | | | | |
| | (mol %) | | | | | | |
| Mw | | 9100 | | 9000 | | 8600 | 7700 |
| Mw/Mn | | 1.54 | | 1.44 | | 1.51 | 1.56 |
| | | Synthesis | | Synthesis | | Synthesis Example | Synthesis |
| | | Example 5 | | Example 6 | | 7 | Example 8 |
| Resin [A] | | A-5 | | A-6 | | A-7 | A-8 |
| Monomer | Type | M-1 | M-4 | M-1 | M-4 | M-1 | M-1 |
| providing | Amount of | 40 | 15 | 40 | 15 | 40 | 40 |
| structural | monomer | | | | | | |
| unit (I) | (mol %) | | | | | | |
| | Proportion | 41.1 | 12.1 | 40.7 | 13.2 | 40.2 | 40.2 |
| | of | | | | | | |
| | structural | | | | | | |
| | unit | | | | | | |
| | (mol %) | | | | | | |

| | | | | | | |
|---|---|---|---|---|---|---|
| Monomer | Type | M-10 | M-11 | M-10 | M-13 | M-7 |
| providing | Amount of | 45 | 45 | 30 | 20 | 40 |
| structural | monomer | | | | | |
| unit (II-1) | (mol %) | | | | | |
| | Proportion | 46.8 | 46.1 | 29.6 | 19.7 | 41.1 |
| | of | | | | | |
| | structural | | | | | |
| | unit | | | | | |
| | (mol %) | | | | | |

| | | | | | |
|---|---|---|---|---|---|
| Monomer | Type | — | — | M-14 | M-15 |
| providing | Amount of | — | — | 10 | 20 |
| structural | monomer | | | | |
| unit (II-2) | (mol %) | | | | |
| | Proportion | — | — | 10.5 | 18.7 |
| | of | | | | |
| | structural | | | | |
| | unit | | | | |
| | (mol %) | | | | |

| | | | | |
|---|---|---|---|---|
| Mw | 7900 | 8100 | 8000 | 8500 |
| Mw/Mn | 1.44 | 1.45 | 1.57 | 1.61 |

| | | Synthesis | Synthesis | Synthesis |
|---|---|---|---|---|
| | | Example | Example | Example |
| | | 9 | 10 | 11 |
| Resin [A] | | A-9 | A-10 | A-11 |
| Monomer | Type | M-1 | M-1 | M-1 |
| providing | Amount of | 50 | 40 | 40 |
| structural | monomer | | | |
| unit (I) | (mol %) | | | |
| | Proportion | 51.0 | 41.3 | 42.8 |
| | of | | | |
| | structural | | | |
| | unit | | | |
| | (mol %) | | | |
| Monomer | Type | M-8 | M-9 | M-6 |
| providing | Amount of | 50 | 60 | 60 |
| structural | monomer | | | |
| unit (II-1) | (mol %) | | | |
| | Proportion | 49.0 | 58.7 | 57.2 |
| | of | | | |
| | structural | | | |
| | unit | | | |
| | (mol %) | | | |
| Monomer | Type | — | — | — |
| providing | Amount of | — | — | — |
| structural | monomer | | | |
| unit (II-2) | (mol %) | | | |
| | Proportion | — | — | — |
| | of | | | |
| | structural | | | |
| | unit | | | |
| | (mol %) | | | |

| | | | |
|---|---|---|---|
| Mw | 7800 | 8200 | 8000 |
| Mw/Mn | 1.55 | 1.55 | 1.43 |

### Synthesis Example 12

### (Synthesis of Resin (A-12))

Monomer (M-1) and monomer (M-18) were dissolved in 1-methoxy-2-propanol (200 parts by mass) so that the ratio by mole was adjusted to 50/50 (mol %). To the solution, AIBN (5 mol %) serving as an initiator was added, to thereby prepare a monomer solution. Separately, 1-methoxy-2-propanol (100 parts by mass) was added to a reaction container. The atmosphere of the container was purged with nitrogen for 30 minutes. Subsequently, the inside temperature of the reaction container was adjusted to 80° C., and the above-prepared monomer solution was added dropwise to the container over 3 hours under stirring. Polymerization reaction was conducted for 6 hours, wherein the time of start of dropwise addition was employed as the time of initiating polymerization reaction. After completion of polymerization reaction, the reaction mixture was water-cooled to 30° C. or lower. The cooled polymer solution was transferred to hexane (2,000 parts by mass), and the precipitated white powder was separated through filtration. The thus-separated white powder was washed twice with hexane, and separated through filtration. The thus-obtained powder was dissolved in 1-methoxy-2-propanol (300 parts by mass). Subsequently, methanol (500 parts by mass), triethylamine (50 parts by mass), and ultrapure water (10 parts by mass) were added to the solution, and hydrolysis reaction was carried out at 70° C. for 6 hours under stirring. After completion of reaction, the residual solvent was distilled off, and the recovered solid was dissolved in acetone (100 parts by mass). The solution was added dropwise to water (500 parts by mass), to thereby solidify the resin. The thus-obtained solid was separated through filtration and dried at 50° C. for 13 hours, to thereby yield resin (A-12) in the form of white powder (yield: 79%). The resin (A-12) was found to have an Mw of 5,200 and an Mw/Mn of 1.60. Through ¹³C-NMR analysis, the ratio of structural units derived from monomer (M-1) and monomer (M-18) was 51.3 mol % and 48.7 mol %, respectively.

### Synthesis Examples 13 to 15

### (Synthesis of Resins (A-13) to (A-15))

The procedure of Synthesis Example 12 was repeated, except that the types and amounts of monomers were changed as shown in Table 2, to thereby yield resins (A-13) to (A-15), respectively. Table 2 also shows structural unit proportions (mol %) and physical properties (Mw and Mw/Mn) of each of the produced resins.

TABLE 2

| | | Synthesis | Synthesis | Synthesis | Synthesis |
|---|---|---|---|---|---|
| | | Example 12 | Example 13 | Example 14 | Example 15 |
| Resin [A] | | A-12 | A-13 | A-14 | A-15 |
| Monomer | Type | M-1 | M-3 | M-2 | M-1 |
| providing | Amount of | 50 | 50 | 50 | 55 |
| structural | monomer | | | | |
| unit (I) | (mol %) | | | | |
| | Proportion of | 51.3 | 47.9 | 48.1 | 55.7 |
| | structural unit | | | | |
| | (mol %) | | | | |
| Monomer | Type | — | M-14 | M-17 | M-17 |
| providing | Amount of | — | 10 | 20 | 15 |
| structural | monomer | | | | |
| unit (II-2) | (mol %) | | | | |
| | Proportion of | — | 10.3 | 21.3 | 15.1 |
| | structural unit | | | | |
| | (mol %) | | | | |
| Monomer | Type | M-18 | M-19 | M-18 | M-19 |
| providing | Amount of | 50 | 40 | 30 | 30 |
| structural | monomer | | | | |
| unit (III) | (mol %) | | | | |
| | Proportion of | 48.7 | 41.8 | 30.6 | 29.2 |
| | structural unit | | | | |
| | (mol %) | | | | |

| | | | | |
|---|---|---|---|---|
| Mw | 5200 | 5500 | 5100 | 6100 |
| Mw/Mn | 1.60 | 1.53 | 1.59 | 1.50 |

### Synthesis Example 16

### (Synthesis of High-Fluorine Content Resin (F-1))

Monomer (M-1) and monomer (M-20) were dissolved in 2-butanone (200 parts by mass) so that the ratio by mole thereof were adjusted to 20/80 (mol %). To the solution, AIBN (4 mol % serving as an initiator was added, to thereby prepare a monomer solution. Separately, 2-butanone (100 parts by mass) was added to a reaction container. The atmosphere of the container was purged with nitrogen for 30 minutes. Subsequently, the inside temperature of the reaction container was adjusted to 80° C., and the above-prepared monomer solution was added dropwise to the container over 3 hours under stirring. Polymerization reaction was conducted for 6 hours, wherein the time of start of dropwise addition was employed as the time of initiating polymerization reaction. After completion of polymerization reaction, the reaction mixture was water-cooled to 30° C. or lower. The solvent was changed to acetonitrile (400 parts by mass). Hexane (100 parts by mass) was added to the mixture under stirring, and the acetonitrile layer was recovered. This procedure was repeated 3 times. The solvent was changed to propylene glycol monomethyl ether acetate, to thereby yield a solution of high-fluorine content resin (E-1) (yield: 69%). The high-fluorine content resin (E-1) was found to have an Mw of 6,000 and an Mw/Mn of 1.62. Through ¹³C-NMR analysis, the proportions of structural units derived from monomer (M-1) and monomer (M-20) were found to be 19.9 mol % and 80.1 mol %, respectively.

### Synthesis Examples 17 to 20

### (Synthesis of High-Fluorine Content Resins (E-2) to (E-5))

The procedure of Synthesis Example 16 was repeated, except that the types and amounts of monomers were changed as in Table 3, to thereby yield high-fluorine content resins (E-2) to (E-5), respectively. Table 3 also shows structural unit proportions (mol %) and physical properties (Mw and Mw/Mn) of each of the high-fluorine content resins produced.

TABLE 3

| | | Synthesis | Synthesis | Synthesis | Synthesis | Synthesis |
|---|---|---|---|---|---|---|
| | | Example | Example | Example | Example | Example |
| | | 16 | 17 | 18 | 19 | 20 |
| Resin [E] | | E-1 | E-2 | E-3 | E-4 | E-5 |
| Monomer | Type | M-20 | M-21 | M-22 | M-22 | M-20 |
| providing | Amount of | 80 | 80 | 60 | 60 | 60 |
| structural | monomer | | | | | |
| unit (F) | (mol %) | | | | | |
| | Proportion of | 80.1 | 81.9 | 61.3 | 60.3 | 59.2 |
| | structural unit | | | | | |
| | (mol %) | | | | | |
| Monomer | Type | M-1 | M-1 | — | M-4 | M-2 |
| providing | Amount of | 20 | 20 | — | 20 | 10 |
| structural | monomer | | | | | |
| unit (I) | (mol %) | | | | | |
| | Proportion of | 19.9 | 18.1 | — | 18.7 | 10.3 |
| | structural unit | | | | | |
| | (mol %) | | | | | |
| Monomer | Type | — | — | — | M-14 | M-17 |
| providing | Amount of | — | — | — | 20 | 30 |
| structural | monomer | | | | | |
| unit (II) | (mol %) | | | | | |
| | Proportion of | — | — | — | 21.0 | 30.5 |
| | structural unit | | | | | |
| | (mol %) | | | | | |
| Monomer | Type | — | — | M-16 | — | — |
| providing | Amount of | — | — | 40 | — | — |
| additional | monomer | | | | | |
| structural | (mol %) | | | | | |
| unit | Proportion of | — | — | 38.7 | — | — |
| | structural unit | | | | | |
| | (mol %) | | | | | |

| | | | | | |
|---|---|---|---|---|---|
| Mw | 6000 | 7200 | 6300 | 7000 | 6100 |
| Mw/Mn | 1.62 | 1.77 | 1.82 | 1.84 | 1.86 |

### <[C] Acid Diffusion Control Agent>

### Synthesis of Acid Diffusion Control Agent [C]

### Synthesis Example 21

### (Synthesis of Compound (C-1-a))

A compound (C-1-a) was synthesized through the following reaction scheme.

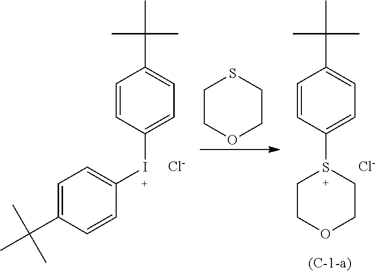

To a reactor, bis(4-(tert-butyl)phenyl)iodonium chloride (20.0 mmol), 1,4-thioxane (20.0 mmol), copper(II) acetate (2.00 mmol), and chloroform (50 g) were added, and the contents were stirred for 24 hours under cooling with ice. Impurity of the product was removed through filtration with Celite, and the solvent was distilled off. The product was purified through recrsyallization, to thereby yield a compound represented by the aforementioned formula (C-1-a) (hereinafter referred to as a “compound (C-1-a)”) at a suitable yield.

### Synthesis Example 22

### (Synthesis of Compound (C-1))

A compound (C-1) was synthesized through the following reaction scheme.

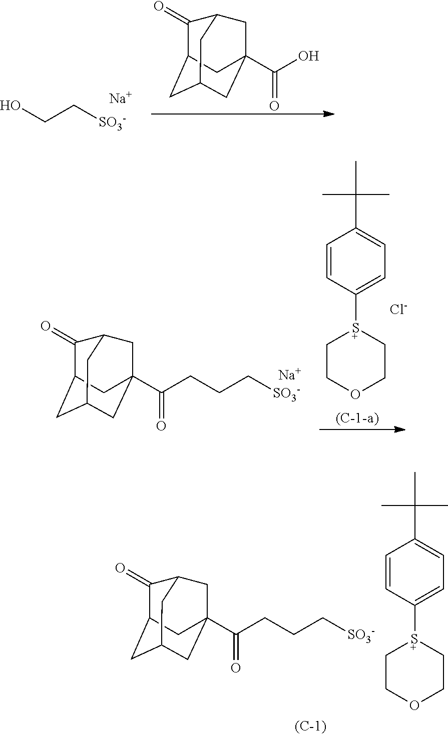

To a reactor, sodium isethionate (20.0 mmol), 2-adamantanone-5-carboxylic acid (20.0 mmol), dicyclohexylcarbodiimide (20.0 mmol), and methylene chloride (50 g) were added, and the contents were stirred at room temperature for 4 hours. Subsequently, water was added to dilute the reaction mixture, and the product was subjected to extraction by adding methylene chloride thereto. The organic layer was separated and was dried over sodium sulfate. The solvent was distilled off, to thereby yield a sodium sulfonate salt compound at a suitable yield.

To the thus-obtained sodium sulfonate salt compound, the compound (C-1-a) (20.0 mmol), and then a mixture of water and dichloromethane (1:3 (ratio by mass)) were added, to thereby prepare a 0.5M solution. The solution was vigorously stirred at room temperature for 3 hours, and dichloromethane was added thereto for extraction. The separated organic layer was dried over sodium sulfate, and the solvent was distilled off, followed by purification through column chromatography, to thereby yield a compound represented by the aforementioned formula (C-1) (hereinafter referred to as a “compound (C-1)”) at a suitable yield.

### Synthesis Examples 23 to 30

### (Synthesis of Compounds (C-2) to (C-9))

The procedure of Synthesis Example 22 was repeated, except that the raw materials and precursor were appropriately altered, to thereby synthesize onium salts represented by the following formulas (C-2) to (C-9) (hereinafter, the onium salts may also be referred to as “compounds (C-2) to (C-9),” respectively).

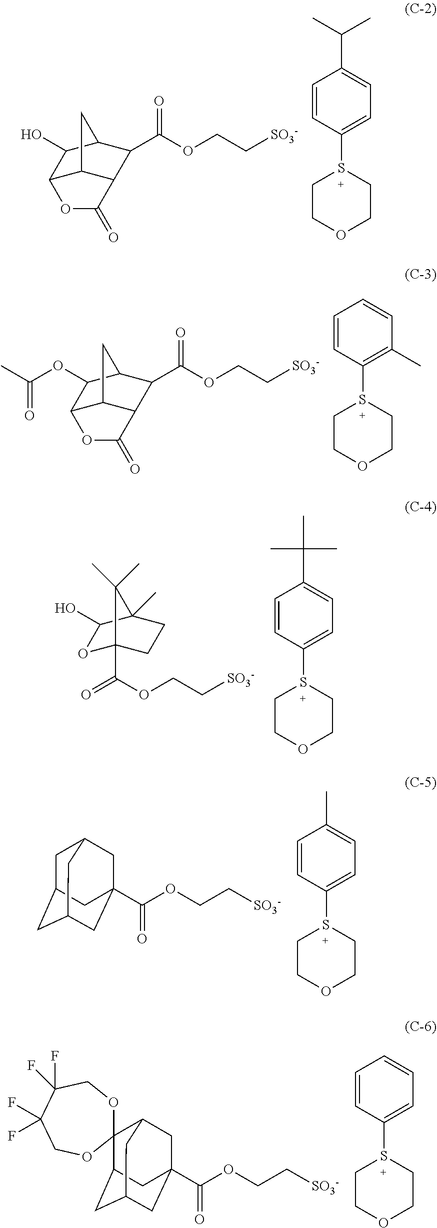

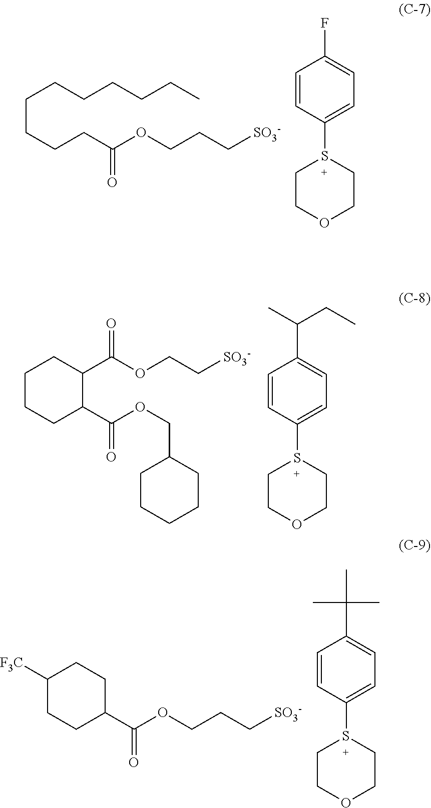

### Synthesis Example 31

### (Synthesis of Compound (C-10))

A compound (C-10) was synthesized through the following reaction scheme.

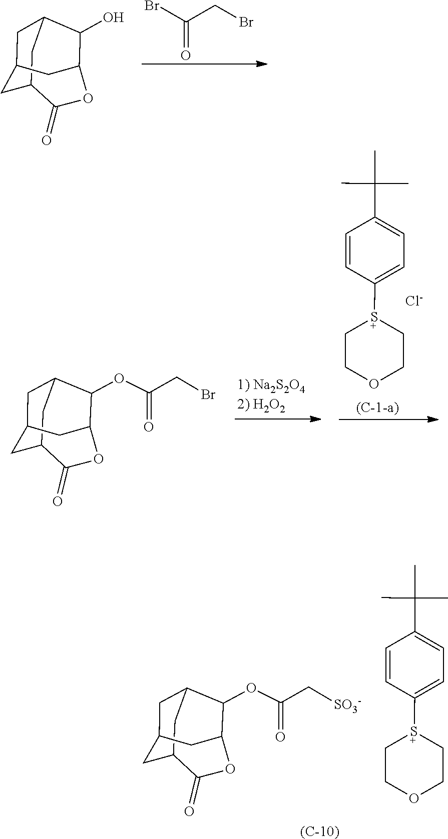

To a reactor, 2-hydroxy-4-oxatricyclo[4.3.1.1]undecan-5-one (20.0 mmol), bromoacetyl bromide (20.0 mmol), triethylamine (20.0 mmol), and tetrahydrofuran (50 g) were added, and the contents were stirred at room temperature for 1 hour. Subsequently, saturated aqueous ammonium chloride was added to the reaction mixture, to thereby terminate reaction. The product was subjected to extraction by adding ethyl acetate thereto. The organic layer was separated, and then washed sequentially with saturated aqueous sodium chloride and water and dried over sodium sulfate. The solvent was distilled off, and purification was conducted through column chromatography, to thereby yield a bromo form at a suitable yield.

To the thus-obtained bromo form, a mixture of acetonitrile and water (1:1 (ratio by mass)) was added, to thereby prepare a 1M solution. Then, sodium dithionite (30.0 mmol) and sodium hydrogen carbonate (30.0 mmol) were added to the above-prepared solution, and the mixture was allowed to react at 70° C. for 4 hours. The reaction mixture was subjected to extraction with acetonitrile, and the solvent was distilled off. Further, a mixture of acetonitrile and water (3:1 (ratio by mass)) was added, to thereby prepare a 0.5M solution. Then, hydrogen peroxide (60.0 mmol) and sodium tungstate (2.00 mmol) were added thereto, and the mixture was stirred at 50° C. for 12 hours under heating. The reaction mixture was subjected to extraction with acetonitrile, and the solvent was distilled off, to thereby yield a sodium sulfonate salt compound. To the above sodium sulfonate salt compound, the intermediate salt represented by the aforementioned (C-1-a) (20.0 mmol), and a mixture of water and dichloromethane (1:3 (ratio by mass)) were added, to thereby prepare a 0.5M solution. The solution was vigorously stirred at room temperature for 3 hours, and dichloromethane was added thereto for extraction. The separated organic layer was dried over sodium sulfate, and the solvent was distilled off, followed by purification through column chromatography, to thereby yield a compound represented by the aforementioned formula (C-10) (hereinafter referred to as a “compound (C-10)”) at a suitable yield.

### Synthesis Examples 32 to 36

### (Synthesis of Compounds (C-11) to (C-15))

The procedure of Synthesis Example 31 was repeated, except that the raw materials and precursor were appropriately altered, to thereby synthesize onium salts represented by the following formulas (C-11) to (C-15) (hereinafter, the onium salts may also be referred to as “compounds (C-11) to (C-15),” respectively).

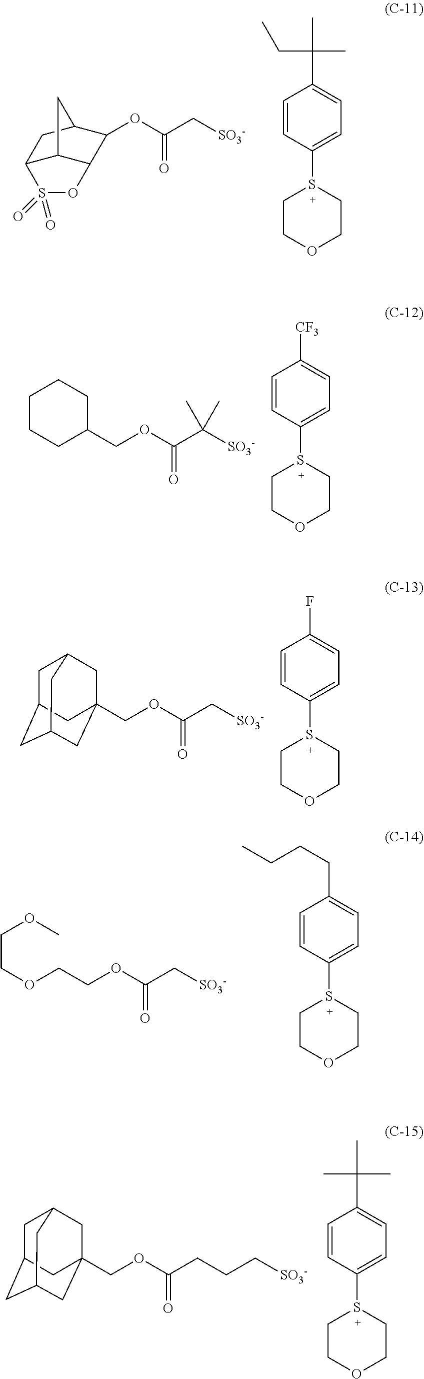

### Synthesis Example 37

### (Synthesis of Compound (C-16))

A compound (C-16) was synthesized through the following reaction scheme.

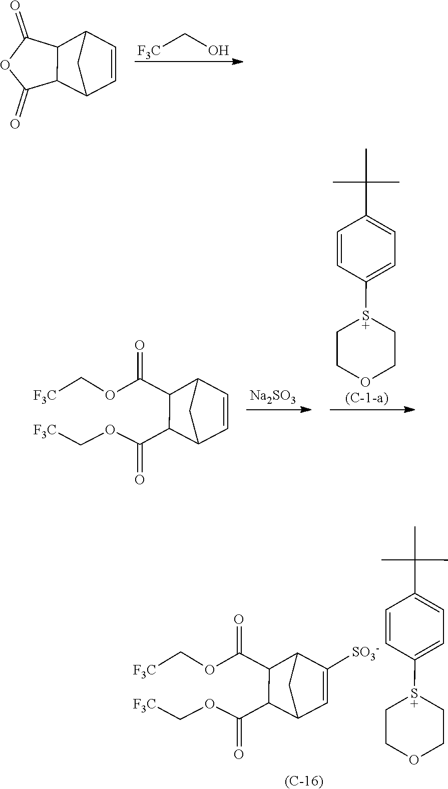

To a reactor, 5-norbornene-2,3-dicarboxylic anhydride (20.0 mmol), 2,2,2-trifluoroethanol (40.0 mmol), triethylamine (60.0 mmol), and tetrahydrofuran (50 g) were added, and the contents were stirred at room temperature for 15 hours. Subsequently, saturated ammonium chloride was added to the reaction mixture, to thereby terminate reaction. The product was subjected to extraction by adding ethyl acetate thereto. The organic layer was separated, and then washed sequentially with saturated aqueous sodium chlorideand dried over sodium sulfate. The solvent was distilled off, and purification was conducted through column chromatography, to thereby yield a diester form at a suitable yield.

To the thus-obtained diester form, a mixture of acetonitrile and water (1:1 (ratio by mass)) was added, to thereby prepare a 1M solution. Then, sodium sulfite (30.0 mmol) was added to the above-prepared solution, and the mixture was allowed to react at 100° C. for 12 hours. The reaction mixture was subjected to extraction with acetonitrile, and the solvent was distilled off, to thereby yield a sodium sulfonate salt compound. To the above sodium sulfonate salt compound, the intermediate salt represented by the aforementioned (C-1-a) (20.0 mmol), and a mixture of water and dichloromethane (1:3 (ratio by mass)) were added, to thereby prepare a 0.5M solution. The solution was vigorously stirred at room temperature for 3 hours, and dichloromethane was added thereto for extraction. The separated organic layer was dried over sodium sulfate, and the solvent was distilled off, followed by purification through column chromatography, to thereby yield a compound represented by the aforementioned formula (C-16) (hereinafter referred to as a “compound (C-16)”) at a suitable yield.

### Synthesis Examples 38 to 46

### (Synthesis of Compounds (C-17) to (C-25))

The procedure of Synthesis Example 37 was repeated, except that the raw materials and precursor were appropriately altered, to thereby synthesize onium salts represented by the following formulas (C-17) to (C-25) (hereinafter, the onium salts may also be referred to as “compounds (C-17) to (C-25),” respectively).

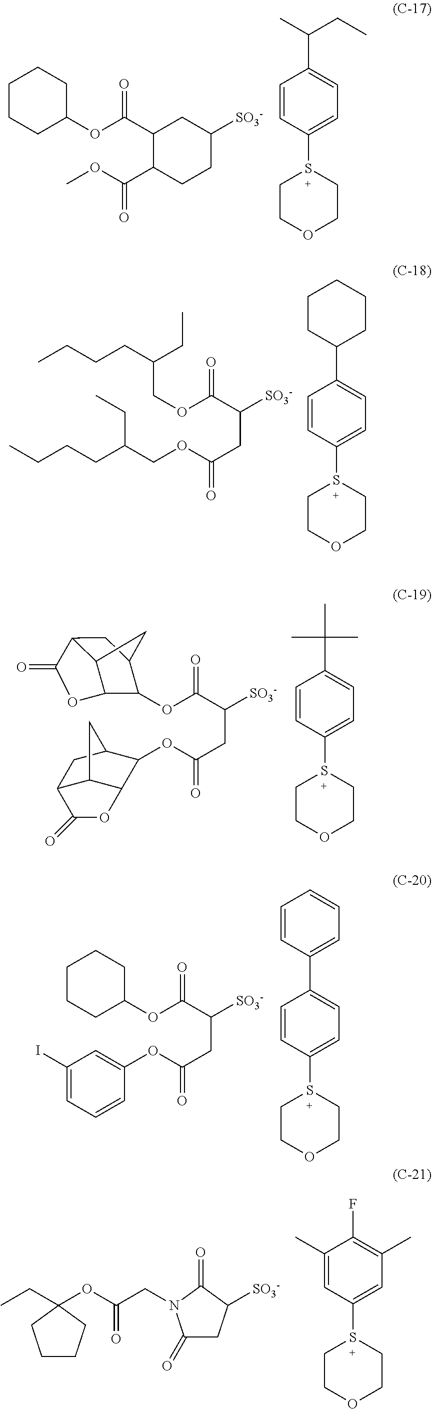

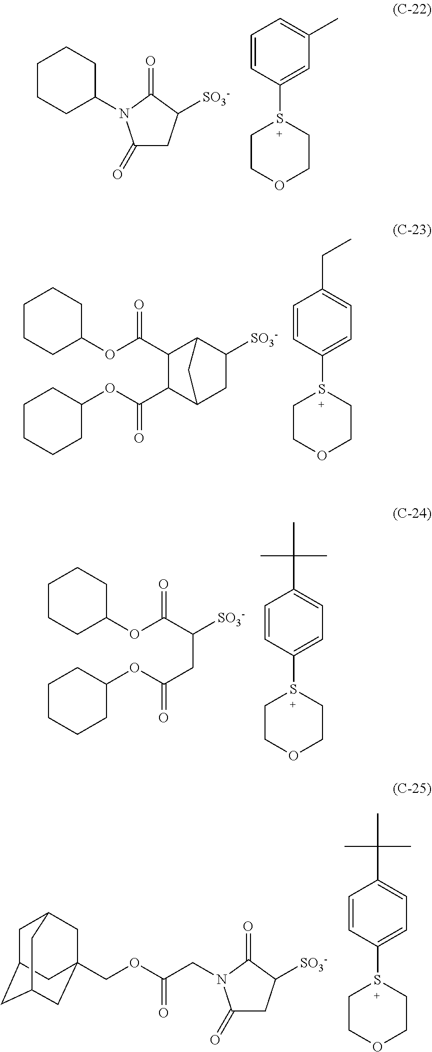

Onium salts other than compounds (C-1) to (C-25) cc-1 to cc-12: Compounds represented by the following formulas (cc-1) to (cc-12) (the compounds represented by the formulas (cc-1) to (cc-12) may also be referred to as “compounds (cc-1) to (cc-12),” respectively.

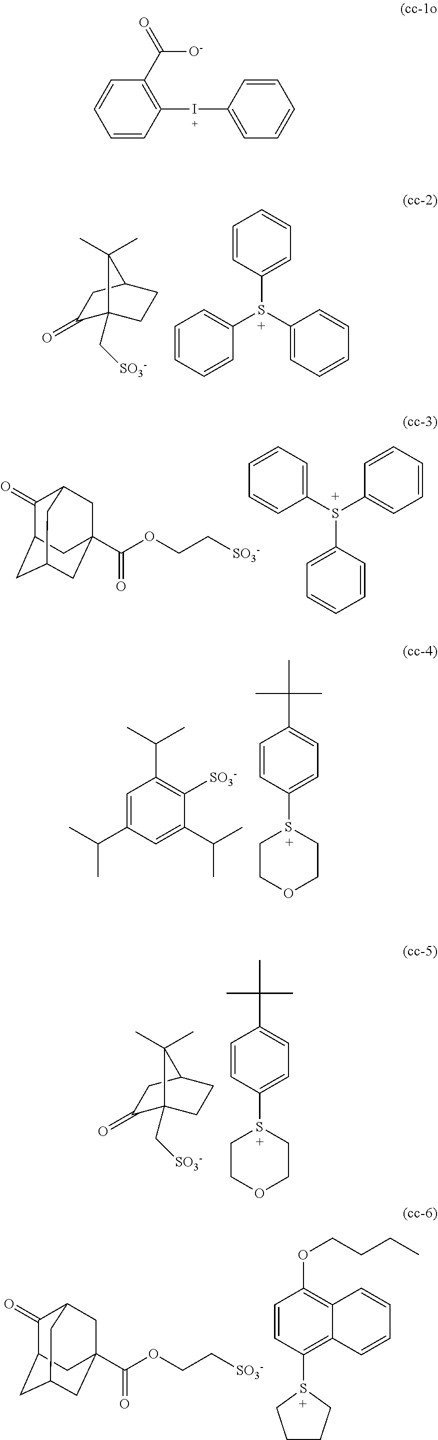

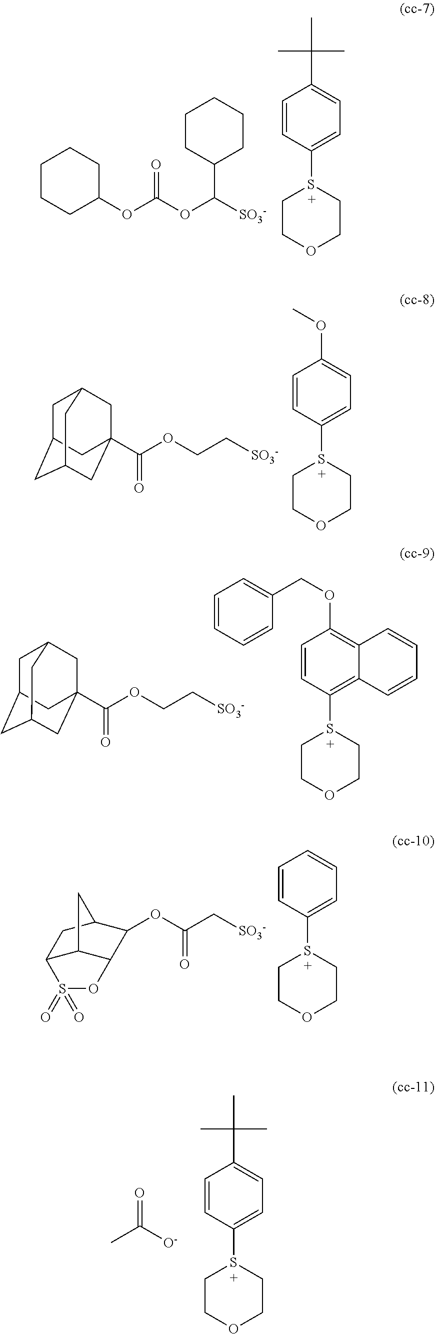

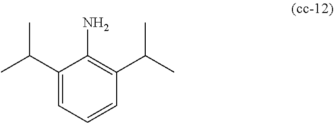

### <Radiation-Sensitive Acid-Generating Agent [B]>

B-1 to B-10: Compounds represented by the following formulas (B-1) to (B-10) (hereinafter, the compounds represented by the formulas (B-1) to (B-10) may also be referred to as “compound (B-1)” to “compound (B-10),” respectively).

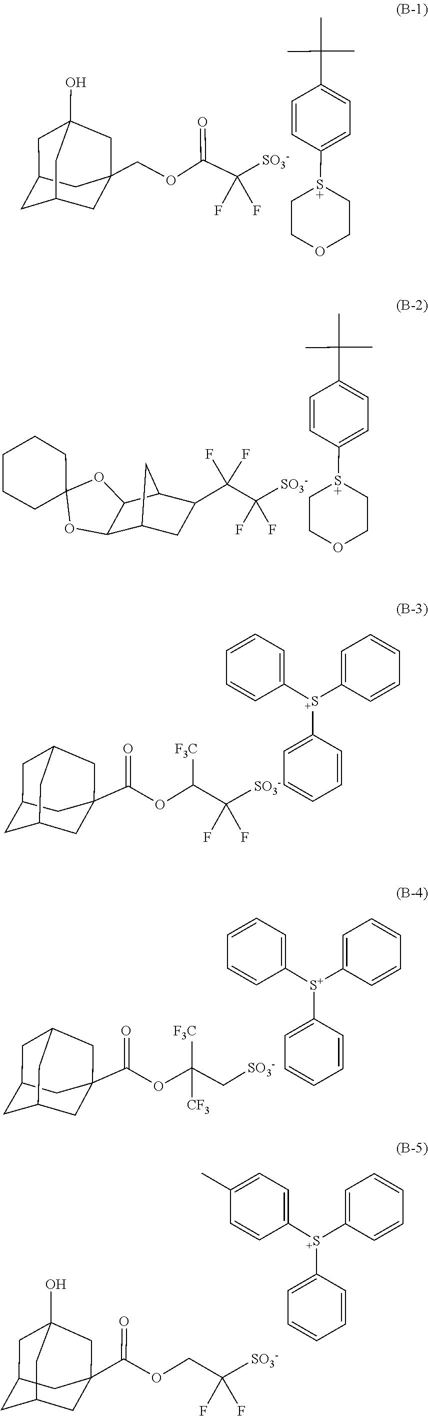

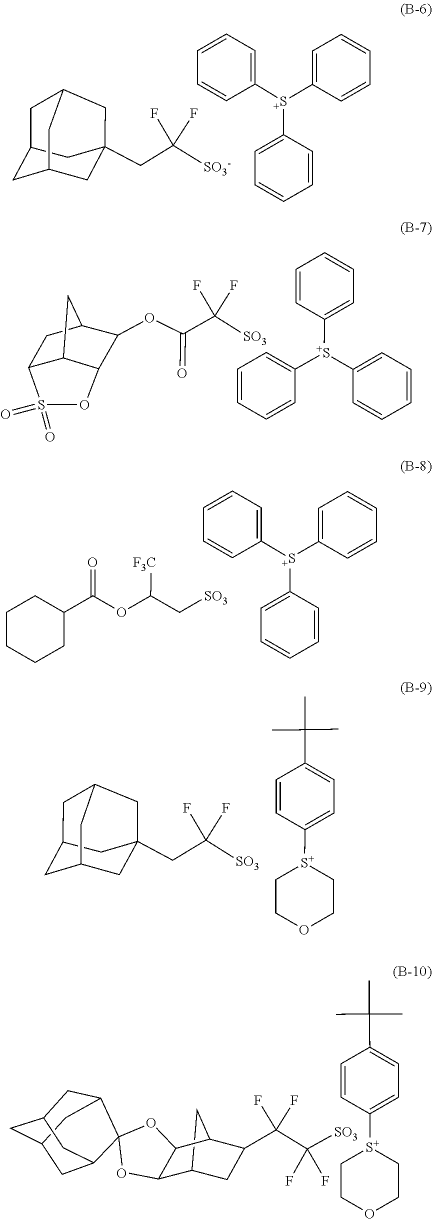

<Solvent [D]>
D-1: Propylene glycol monomethyl ether acetate
D-2: Propylene glycol monomethyl ether
D-3: γ-Butyrolactone
D-4: Ethyl lactate

### <Preparation of Negative-Type Radiation-Sensitive Resin Composition for Exposure to ArF Light>

### Example 1

(A-1) serving as the resin [A] (100 parts by mass), (B-1) serving as the radiation-sensitive acid-generating agent [B](10.0 parts by mass), (C-1) serving as the acid diffusion control agent [C] (8.0 parts by mass), (E-1) serving as the high-fluorine content resin [E] (3.0 parts by mass (solid content)), and a (D-1)/(D-2)/(D-3) mixed solvent serving as the solvent [D] (3,230 parts by mass (2,240/960/30 (parts by mass)) were mixed together, and the mixture was filtered through a membrane filter (pore size: 0.2 μm), to thereby prepare a radiation-sensitive resin composition (J-1).

### Examples 2 to 59 and Comparative Examples 1 to 12

The procedure of Example 1 was repeated, except that the types and amounts of the components were changed as shown in Tables 4 and 5 below, to thereby prepare radiation-sensitive resin compositions (J-2) to (J-59), and (CJ-1) to (CJ-12).

TABLE 4

| | | Example 1 | Example 2 | Example 3 | Example 4 | Example 5 | Example 6 |
|---|---|---|---|---|---|---|---|
| Radiation-sensitive | | J-1 | J-2 | J-3 | J-4 | J-5 | J-6 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-1 | C-2 | C-3 | C-4 | C-5 | C-6 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | | | | Example | Example | Example |
| | | Example 7 | Example 8 | Example 9 | 10 | 11 | 12 |
| Radiation-sensitive | | J-7 | J-8 | J-9 | J-10 | J-11 | J-12 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-7 | C-8 | C-9 | C-10 | C-11 | C-12 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 13 | 14 | 15 | 16 | 17 | 18 |
| Radiation-sensitive | | J-13 | J-14 | J-15 | J-16 | J-17 | J-18 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-13 | C-14 | C-15 | C-16 | C-17 | C-18 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 19 | 20 | 21 | 22 | 23 | 24 |
| Radiation-sensitive | | J-19 | J-20 | J-21 | J-22 | J-23 | J-24 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-19 | C-20 | C-21 | C-22 | C-23 | C-24 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 25 | 26 | 27 | 28 | 29 | 30 |
| Radiation-sensitive | | J-25 | J-26 | J-27 | J-28 | J-29 | J-30 |
| resin composition | | | | | | | |
| Resin [A] | Type | A-1 | A-2 | A-3 | A-4 | A-5 | A-6 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent [B] | (part(s) | | | | | | |
| | by mass) | | | | | | |
| Acid | Type | C-25 | C-1 | C-1 | C-1 | C-1 | C-1 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 31 | 32 | 33 | 34 | 35 | 36 |
| Radiation-sensitive | | J-31 | J-32 | J-33 | J-34 | J-35 | J-36 |
| resin composition | | | | | | | |
| Resin [A] | Type | A-7 | A-8 | A-9 | A-10 | A-11 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-2 |
| generating | Amount | 10.0 | 10.0 | 10.0 | 10.0 | 10.0 | 10.0 |
| agent [B] | (part(s) | | | | | | |
| | by mass) | | | | | | |
| Acid | Type | C-1 | C-1 | C-1 | C-1 | C-1 | C-1 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |

| | | Example 37 | Example 38 | Example 39 | Example 40 |
|---|---|---|---|---|---|
| Radiation-sensitive | | J-37 | J-38 | J-39 | J-40 |
| resin composition | | | | | |
| Resin [A] | Type | A-1 | A-1 | A-1 | A-1 |
| | Amount | 100 | 100 | 100 | 100 |
| | (part(s) | | | | |
| | by | | | | |
| | mass) | | | | |
| Acid- | Type | B-3 | B-4 | B-5 | B-6 |
| generating | Amount | 10.0 | 10.0 | 10.0 | 10.0 |
| agent [B] | (part(s) | | | | |
| | by | | | | |
| | mass) | | | | |
| Acid | Type | C-1 | C-1 | C-1 | C-1 |
| diffusion | Amount | 8.0 | 8.0 | 8.0 | 8.0 |
| control | (part(s) | | | | |
| agent [C] | by | | | | |
| | mass) | | | | |
| Resin [E] | Type | E-1 | E-1 | E-1 | E-1 |
| | Amount | 3.0 | 3.0 | 3.0 | 3.0 |
| | (part(s) | | | | |
| | by | | | | |
| | mass) | | | | |
| Solvent | Type | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 |
| | (part(s) | | | | |
| | by | | | | |
| | mass) | | | | |

TABLE 5

| | | Example | Example | Example | Example | Example | Example |
|---|---|---|---|---|---|---|---|
| | | 41 | 42 | 43 | 44 | 45 | 46 |
| Radiation-sensitive | | J-41 | J-42 | J-43 | J-44 | J-45 | J-46 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-7 | B-8 | B-9 | B-10 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-1 | C-1 | C-1 | C-1 | C-1 | C-1 |
| diffusion | Amount | 8.0 | 8.0 | 8.0 | 8.0 | 8.0 | 8.0 |
| control | (part(s) | | | | | | |
| agent [C] | by mass) | | | | | | |
| Resin [E] | Type | E-1 | E-1 | E- | E-1 | E-2 | E-3 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 47 | 48 | 49 | 50 | 51 | 52 |
| Radiation-sensitive | | J-47 | J-48 | J-49 | J-50 | J-51 | J-52 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1 | B-1 | B-1 | B-1 |
| generating | Amount | 10.0 | 10.0 | 10.0 | 10.0 | 10.0 | 10.0 |
| agent | (part(s) | | | | | | |
| [B] | by mass) | | | | | | |
| Acid | Type | C-1 | C-1 | C-1 | C-1 | C-1/C-11 | C-1/C-16 |
| diffusion | Amount | 8.0 | 0.5 | 4.0 | 15.0 | 4.0/4.0 | 4.0/4.0 |
| control | (part(s) | | | | | | |
| agent [C] | by mass) | | | | | | |
| Resin [E] | Type | E-4 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |
| | | Example | Example | Example | Example | Example | Example |
| | | 53 | 54 | 55 | 56 | 57 | 58 |
| Radiation-sensitive | | J-53 | J-54 | J-55 | J-56 | J-57 | J-58 |
| resin composition | | | | | | | |
| Resin [A] | Type | A-1 | A-1 | A-1 | A-1 | A-1 | A-1 |
| | Amount | 100 | 100 | 100 | 100 | 100 | 100 |
| | (part(s) | | | | | | |
| | by mass) | | | | | | |
| Acid- | Type | B-1 | B-1 | B-1/B-2 | B-1/B-5 | B-1/B-7 | B-8/B-9 |
| generating | Amount | 10.0 | 10.0 | 5.0/5.0 | 5.0/5.0 | 5.0/5.0 | 5.0/5.0 |
| agent | (part(s) | | | | | | |
| [B] | by mass) | | | | | | |
| Acid | Type | C-4/C-21 | C-1/cc-1 | C-1 | C-1 | C-1 | C-9/C-25 |
| diffusion | Amount | 4.0/4.0 | 4.0/4.0 | 8.0 | 8.0 | 8.0 | 4.0/4.0 |
| control | (part(s) | | | | | | |
| agent [C] | by mass) | | | | | | |
| Resin [E] | Type | E-1 | E-1 | E-1 | E-1 | E-1 | E-1 |
| | Amount | 3.0 | 3.0 | 3.0 | 3.0 | 3.0 | 3.0 |
| | (part(s) | | | | | | |
| | by mass) | | | | | | |
| Solvent | Type | D-1/D-2/D-3 | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 | 2240/960/30 |
| | (part(s) | | | | | | |
| | by mass) | | | | | | |

| | | | Comparative | Comparative | Comparative | Comparative |
|---|---|---|---|---|---|---|
| | | Example 59 | Example 1 | Example 2 | Example 3 | Example 4 |
| Radiation-sensitive | | J-59 | CJ-1 | CJ-2 | CJ-3 | CJ-4 |
| resin composition | | | | | | |
| Resin [A] | Type | A-1 | A-1 | A-1 | A-1 | A-1 |
| | Amount | 100 | 100 | 100 | 100 | 100 |
| | (part(s) | | | | | |
| | by mass) | | | | | |
| Acid- | Type | B-4/B-10 | B-1 | B-1 | B-1 | B-1 |
| generating | Amount | 5.0/5.0 | 10.0 | 10.0 | 10.0 | 10.0 |
| agent [B] | (part(s) | | | | | |
| | by mass) | | | | | |
| Acid | Type | C-6/C-24 | cc-1 | cc-2 | cc-3 | cc-4 |
| diffusion | Amount | 4.0/4.0 | 8.0 | 8.0 | 8.0 | 8.0 |
| control | (part(s) | | | | | |
| agent [C] | by mass) | | | | | |
| Resin [E] | Type | E-1 | E-1 | E-1 | E-1 | E-1 |
| | Amount | 3.0 | 3.0 | 3.0 | 3.0 | 3.0 |
| | (part(s) | | | | | |
| | by mass) | | | | | |
| Solvent | Type | D-1/D-2/D-3 | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 | 2240/960/30 |
| | (part(s) | | | | | |
| | by mass) | | | | | |

| | | Comparative | Comparative | Comparative | Comparative |
|---|---|---|---|---|---|
| | | Example 5 | Example 6 | Example 7 | Example 8 |
| Radiation-sensitive | | CJ-5 | CJ-6 | CJ-7 | CJ-8 |
| resin composition | | | | | |
| Resin [A] | Type | A-1 | A-1 | A-1 | A-1 |
| | Amount | 100 | 100 | 100 | 100 |
| | (part(s) | | | | |
| | by mass) | | | | |
| Acid- | Type | B-1 | B-1 | B-1 | B-1 |
| generating | Amount | 10.0 | 10.0 | 10.0 | 10.0 |
| agent [B] | (part(s) | | | | |
| | by mass) | | | | |
| Acid | Type | cc-5 | cc-6 | cc-7 | cc-8 |
| diffusion | Amount | 8.0 | 8.0 | 8.0 | 8.0 |
| control | (part(s) | | | | |
| agent [C] | by mass) | | | | |
| Resin [E] | Type | E-1 | E-1 | E-1 | E-1 |
| | Amount | 3.0 | 3.0 | 3.0 | 3.0 |
| | (part(s) | | | | |
| | by mass) | | | | |
| Solvent | Type | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 |
| | (part(s) | | | | |
| | by mass) | | | | |
| | | Comparative | Comparative | Comparative | Comparative |
| | | Example 9 | Example 10 | Example 11 | Example 12 |
| Radiation-sensitive | | CJ-9 | CJ-10 | CJ-11 | CJ-12 |
| resin composition | | | | | |
| Resin [A] | Type | A-1 | A-1 | A-1 | A-1 |
| | Amount | 100 | 100 | 100 | 100 |
| | (part(s) | | | | |
| | by | | | | |
| | mass) | | | | |
| Acid- | Type | B- | B-1 | B-1 | B-1 |
| generating | Amount | 10.0 | 10.0 | 10.0 | 10.0 |
| agent [B] | (part(s) | | | | |
| | by | | | | |
| | mass) | | | | |
| Acid | Type | cc-9 | cc-10 | cc-11 | cc-12 |
| diffusion | Amount | 8.0 | 8.0 | 8.0 | 8.0 |
| control | (part(s) | | | | |
| agent [C] | by | | | | |
| | mass) | | | | |
| Resin [E] | Type | E-1 | E-1 | E-1 | E-1 |
| | Amount | 3.0 | 3.0 | 3.0 | 3.0 |
| | (part(s) | | | | |
| | by | | | | |
| | mass) | | | | |
| Solvent | Type | D-1/D-2/D-3 | D-1/D-2/D-3 | D-1/D-2/D-3 | D-1/D-2/D-3 |
| [D] | Amount | 2240/960/30 | 2240/960/30 | 2240/960/30 | 2240/960/30 |
| | (part(s) | | | | |
| | by | | | | |
| | mass) | | | | |

### <Formation of Resist Pattern by Use of Negative-Type Radiation-Sensitive Resin Composition for Exposure to ArF Light>

An underlayer-forming composition (“ARC66,” product of Brewer Science, Inc.) was applied onto a 12-inch silicon wafer by means of a spin coater (“CLEAN TRACK ACT12,” product of Tokyo Electron Limited), and heated at 205° C. for 60 seconds, to thereby form an underlayer film having an average thickness of 100 nm. Onto the thus-formed underlayer film, each of the above-prepared negative-type radiation-sensitive resin compositions for exposure to ArF light was applied by means of the aforementioned spin coater, and heated at 100° C. for 60 seconds for PB (pre-baking). Subsequently, the PB product was cooled at 23° C. for 30 seconds, to thereby form a resist film having an average thickness of 90 nm. Then, the resist film was irradiated with laser light by means of an ArF excimer laser liquid immersion light exposure device (“TWINSCAN XT-1900i,” product of ASML, NA=1.35, lighting condition: dipole (σ=0.9/0.7) through a mask pattern (40 nm hole and 120 nm pitch). After the light exposure, the resist film was subjected to PEB (post exposure baking) at 100° C. for 60 seconds. Then, organic solvent development of the resist film was performed by use of n-butyl acetate serving as an organic solvent developer, and drying was conducted, to thereby form a negative-type resist pattern (40 nm hole and 120 nm pitch).

### <Evaluation>

The resist patterns formed by use of the aforementioned negative-type radiation-sensitive resin compositions for exposure to ArF light were evaluated in terms of sensitivity, CDU performance, focal depth, pattern rectangularity, and storage stability, through the following procedures. Tables 6 and 7 show the results. The measurement of the resist pattern was conducted by means of a scanning electron microscope (“CG-5000,” product of Hitachi High-Tech Corporation).

### [Sensitivity]

In formation of a resist pattern by use of the aforementioned negative-type radiation-sensitive resin composition for exposure to ArF light, a dose which can form a 40-nm hole pattern was employed as an optimum dose, serving as a sensitivity (mJ/cm²). A sensitivity of 30 mJ/cm² or lower was evaluated as “good,” and a sensitivity in excess of 30 mJ/cm² was evaluated as “bad.”

### [Cdu Performance]

A resist pattern (40 nm hole and 105 nm pitch) was observed under the aforementioned scanning electron microscope. The hole size was measured from above at 1,800 points at random, and variation in size (3σ) was determined. The 3σ value in the size was employed as an CDU performance index (nm). Regarding CDU performance, the smaller the CDU performance index, the smaller the variation in hole diameter in a long period (i.e., the more favorable). CDU performance was evaluated as “good” when the 36 was 3.0 nm or less, and as “bad” when the 36 was in excess of 3.0 nm.

### [Focal Depth]

In the resist pattern defined by the optimum dose determined in the aforementioned sensitivity evaluation, the pattern size was measured, while the focus was varied in the depth direction. The margin in the depth direction which allowed that the pattern size fell within a range of 90% to 110% of the standard without generating bridge or residue was determined. The value was employed as a focal depth (nm) index. Regarding focal depth, the greater the focal depth index, the more favorable the focal depth. Focal depth was evaluated as “good” when the index value was 100 nm or greater, and as “bad” when the index value was smaller than 100 nm.

### [Pattern Rectangularity]

The 40-nm hole resist pattern formed through irradiation at an optimum dose determined in the aforementioned sensitivity evaluation was observed under the aforementioned scanning electron microscope. The rectangularity of the resist pattern was evaluated on the basis of the ratio of the length of the upper side to that of the lower side in the shape of a cross-section. Rectangularity was evaluated as “A (very good)” when the ratio was 1.00 to 1.05; “B (good)” when the ratio was greater than 1.05 and 1.10 or smaller; and “C (bad)” when the ratio was greater than 1.10.

### [Storage Stability]

The aforementioned negative-type radiation-sensitive resin compositions for exposure to ArF light were stored at 35° C. for 30 days. Thereafter, the optimum dose which can form a 40-nm hole pattern (i.e., sensitivity) was determined again. When a drop in sensitivity after storage for 30 days was 0% to 1.0%, storage stability was evaluated as “A” (very good). When the drop in sensitivity was greater than 1.0% and 2.0% or smaller, storage stability was evaluated as “B” (good). When the drop in sensitivity was greater than 2.0%, storage stability was evaluated as “C” (bad).

TABLE 6

| | Radiation- | | | | | |
|---|---|---|---|---|---|---|
| | sensitive | Sen- | | Focal | Pattern | |
| | resin | sitivity | CDU | depth | rectan- | Storage |
| | composition | (mJ/cm²) | (nm) | (nm) | gularity | stability |
| Example 1 | J-1 | 25 | 2.4 | 130 | A | A |
| Example 2 | J-2 | 22 | 2.6 | 120 | A | A |
| Example 3 | J-3 | 26 | 2.7 | 130 | A | A |
| Example 4 | J-4 | 28 | 2.5 | 120 | A | A |
| Example 5 | J-5 | 27 | 2.5 | 120 | A | A |
| Example 6 | J-6 | 27 | 2.6 | 120 | A | A |
| Example 7 | J-7 | 22 | 2.7 | 120 | A | A |
| Example 8 | J-8 | 26 | 2.8 | 130 | A | A |
| Example 9 | J-9 | 28 | 2.7 | 130 | A | A |
| Example 10 | J-10 | 27 | 2.7 | 140 | A | A |
| Example 11 | J-11 | 27 | 2.4 | 120 | A | A |
| Example 12 | J-12 | 26 | 2.5 | 130 | A | A |
| Example 13 | J-13 | 25 | 2.7 | 120 | A | A |
| Example 14 | J-14 | 26 | 2.6 | 130 | A | A |
| Example 15 | J-15 | 25 | 2.6 | 120 | A | A |
| Example 16 | J-16 | 20 | 2.5 | 120 | A | A |
| Example 17 | J-17 | 23 | 2.7 | 120 | A | A |
| Example 18 | J-18 | 28 | 2.5 | 120 | A | A |
| Example 19 | J-19 | 25 | 2.8 | 130 | A | A |
| Example 20 | J-20 | 23 | 2.4 | 120 | A | A |
| Example 21 | J-21 | 24 | 2.3 | 120 | A | A |
| Example 22 | J-22 | 23 | 2.3 | 120 | A | A |
| Example 23 | J-23 | 24 | 2.2 | 130 | A | A |
| Example 24 | J-24 | 25 | 2.4 | 120 | A | A |
| Example 25 | J-25 | 24 | 2.5 | 120 | A | A |
| Example 26 | J-26 | 23 | 2.5 | 120 | A | A |
| Example 27 | J-27 | 27 | 2.5 | 120 | A | A |
| Example 28 | J-28 | 26 | 2.4 | 120 | A | A |
| Example 29 | J-29 | 28 | 2.5 | 130 | A | A |
| Example 30 | J-30 | 24 | 2.5 | 130 | A | A |
| Example 31 | J-31 | 26 | 2.6 | 120 | A | A |
| Example 32 | J-32 | 26 | 2.7 | 130 | A | A |
| Example 33 | J-33 | 23 | 2.4 | 130 | A | A |
| Example 34 | J-34 | 22 | 2.5 | 130 | A | A |
| Example 35 | J-35 | 26 | 2.6 | 120 | A | A |
| Example 36 | J-36 | 28 | 2.4 | 120 | A | A |
| Example 37 | J-37 | 23 | 2.5 | 120 | A | A |
| Example 38 | J-38 | 28 | 2.4 | 120 | A | A |
| Example 39 | J-39 | 23 | 2.7 | 120 | A | A |
| Example 40 | J-40 | 27 | 2.4 | 130 | A | A |

TABLE 7

| | Radiation- | | | | | |
|---|---|---|---|---|---|---|
| | sensitive | | | Focal | Pattern | |
| | resin | Sensitivity | CDU | depth | rectan- | Storage |
| | composition | (mJ/cm²) | (nm) | (nm) | gularity | stability |
| Example 41 | J-41 | 21 | 2.6 | 120 | A | A |
| Example 42 | J-42 | 28 | 2.7 | 120 | A | A |
| Example 43 | J-43 | 21 | 2.5 | 120 | A | A |
| Example 44 | J-44 | 24 | 2.2 | 120 | A | A |
| Example 45 | J-45 | 26 | 2.4 | 130 | A | A |
| Example 46 | J-46 | 25 | 2.4 | 130 | A | A |
| Example 47 | J-47 | 25 | 2.4 | 130 | A | A |
| Example 48 | J-48 | 22 | 2.6 | 110 | A | A |
| Example 49 | J-49 | 24 | 2.8 | 120 | A | A |
| Example 50 | J-50 | 27 | 2.7 | 130 | A | A |
| Example 51 | J-51 | 27 | 2.5 | 120 | A | A |
| Example 52 | J-52 | 25 | 2.6 | 130 | A | A |
| Example 53 | J-53 | 28 | 2.7 | 130 | A | A |
| Example 54 | J-54 | 27 | 2.9 | 130 | A | A |
| Example 55 | J-55 | 27 | 2.5 | 130 | A | A |
| Example 56 | J-56 | 23 | 2.3 | 130 | A | A |
| Example 57 | J-57 | 22 | 2.4 | 120 | A | A |
| Example 58 | J-58 | 23 | 2.5 | 130 | A | A |
| Example 59 | J-59 | 24 | 2.4 | 120 | A | A |
| Comparative | CJ-1 | 27 | 3.2 | 30 | B | A |
| Example 1 | | | | | | |
| Comparative | CJ-2 | 28 | 3.3 | 70 | C | A |
| Example 2 | | | | | | |
| Comparative | CJ-3 | 28 | 3.4 | 80 | C | A |
| Example 3 | | | | | | |
| Comparative | CJ-4 | 36 | 3.2 | 70 | A | C |
| Example 4 | | | | | | |
| Comparative | CJ-5 | 32 | 3.5 | 60 | A | C |
| Example 5 | | | | | | |
| Comparative | CJ-6 | 42 | 4.2 | 30 | B | C |
| Example 6 | | | | | | |
| Comparative | CJ-7 | 32 | 3.2 | 70 | A | C |
| Example 7 | | | | | | |
| Comparative | CJ-8 | 35 | 3.6 | 60 | A | C |
| Example 8 | | | | | | |
| Comparative | CJ-9 | 37 | 3.2 | 50 | B | B |
| Example 9 | | | | | | |
| Comparative | CJ-10 | 35 | 3.5 | 60 | B | B |
| Example 10 | | | | | | |
| Comparative | CJ-11 | 42 | 4.5 | 30 | C | C |
| Example 11 | | | | | | |
| Comparative | CJ-12 | 40 | 3.9 | 50 | C | A |
| Example 12 | | | | | | |

As is clear from Tables 6 and 7, the radiation-sensitive resin compositions of Examples 1 to 59 were found to exhibit suitable sensitivity, CDU performance, focal depth, pattern rectangularity, and storage stability, when they were employed in exposure to ArF light. In contrast, in the radiation-sensitive resin compositions of Comparative Examples 1 to 12, one or more properties of sensitivity, CDU performance, focal depth, pattern rectangularity, and storage stability were inferior to those obtained in Examples 1 to 59. In particular, CDU performance and focal depth of Comparative Examples 1 to 12 were found to be inferior to those obtained in Examples 1 to 59. Therefore, by use of the radiation-sensitive resin compositions of Examples 1 to 59 in negative-type exposure to ArF light, high sensitivity and suitable CDU performance and pattern rectangularity can be attained, and the compositions have excellent storage stability.

### <Preparation of Positive-Type Radiation-Sensitive Resin Composition for Exposure to Extreme UV (EUV) Ray>

### Example 60

(A-12) serving as the resin [A] (100 parts by mass), (B-1) serving as the radiation-sensitive acid-generating agent [B] (16.0 parts by mass), (C-1) serving as the acid diffusion control agent [C] (10.0 parts by mass), (E-5) serving as the high-fluorine content resin [E] (3.0 parts by mass (solid content)), and a (D-1)/(D-4) mixed solvent serving as the solvent [D] (6,110 parts by mass (4,280/1,830 (parts by mass)) were mixed together, and the mixture was filtered through a membrane filter (pore size: 0.2 μm), to thereby prepare a positive-type radiation-sensitive resin composition for exposure to EUV (J-60).

### Examples 61 to 72 and Comparative Examples 13 to 18

The procedure of Example 60 was repeated, except that the types and amounts of the components were changed as shown in Table 8 below, to thereby prepare radiation-sensitive resin compositions (J-61) to (J-72), and (CJ-13) to (CJ-18).

TABLE 8

| | | Example | Example | Example | Example | Example | Example | Example |
|---|---|---|---|---|---|---|---|---|
| | | 60 | 61 | 62 | 63 | 64 | 65 | 66 |
| Radiation-sensitive | | J-60 | J-61 | J-62 | J-63 | J-64 | J-65 | J-66 |
| resin composition | | | | | | | | |
| Resin [A] | Type | A-12 | A-12 | A-12 | A-12 | A-12 | A-12 | A-12 |
| | Amount | 100 | 100 | 100 | 100 | 100 | 100 | 100 |
| | (part(s) | | | | | | | |
| | by | | | | | | | |
| | mass) | | | | | | | |
| Acid- | Type | B-1 | B-1 | B-1 | B-1 | B-1 | B-1 | B-1 |
| generating | Amount | 16.0 | 16.0 | 16.0 | 16.0 | 16.0 | 16.0 | 16.0 |
| agent | (part(s) | | | | | | | |
| [B] | by | | | | | | | |
| | mass) | | | | | | | |
| Acid | Type | C-1 | C-4 | C-10 | C-11 | C-16 | C-19 | C-20 |
| diffusion | Amount | 10.0 | 10.0 | 10.0 | 10.0 | 10.0 | 10.0 | 10.0 |
| control | (part(s) | | | | | | | |
| agent [C] | by | | | | | | | |
| | mass) | | | | | | | |
| Resin [E] | Type | E-5 | E-5 | E-5 | E-5 | E-5 | E-5 | E-5 |
| | Amount | 3.0 | 3.0 | 3.0 | 3.0 | 3.0 | 3.0 | 3.0 |
| | (part(s) | | | | | | | |
| | by | | | | | | | |
| | mass) | | | | | | | |
| Solvent | Type | D-1/D-4 | D-1/D-4 | D-1/D-4 | D-1/D-4 | D-1/D-4 | D-1/D-4 | D-1/D-4 |
| [D] | Amount | 4280/1830 | 4280/1830 | 4280/1830 | 4280/1830 | 4280/1830 | 4280/1830 | 4280/1830 |
| | (part(s) | | | | | | | |
| | by | | | | | | | |
| | mass) | | | | | | | |
| | | | | | | | | Comparative |
| | | Example | Example | Example | Example | Example | Example | Example |
| | | 67 | 68 | 69 | 70 | 71 | 72 | 13 |
| Radiation-sensitive | | J-67 | J-68 | J-69 | J-70 | J-71 | J-72 | CJ-13 |
| resin composition | | | | | | | | |
| Resin [A] | Type | A-13 | A-14 | A-15 | A-12 | A-12 | A-12 | A-12 |
| | Amount | 100 | 100 | 100 | 100 | 100 | 100 | 100 |
| | (part(s) | | | | | | | |
| | by | | | | | | | |
| | mass | | | | | | | |
| Acid- | Type | B-1 | B-1 | B-1 | B-3 | B-7 | B-2/B-6 | B-1 |
| generating | Amount | 16.0 | 16.0 | 16.0 | 16.0 | 16.0 | 8.0/8.0 | 16.0 |
| agent | (part(s) | | | | | | | |
| [B] | by | | | | | | | |
| | mass) | | | | | | | |
| Acid | Type | C-1 | C-1 | C-1 | C-1 | C-1 | C-1 | cc-3 |
| diffusion | Amount | 10.0 | 10.0 | 10.0 | 10.0 | 10.0 | 10.0 | 10.0 |
| control | (part(s) | | | | | | | |
| agent [C] | by | | | | | | | |
| | mass) | | | | | | | |
| Resin [E] | Type | E-5 | E-5 | E-5 | E-5 | E-5 | E-5 | E-5 |
| | Amount | 3.0 | 3.0 | 3.0 | 3.0 | 3.0 | 3.0 | 3.0 |
| | (part(s) | | | | | | | |
| | by | | | | | | | |
| | mass) | | | | | | | |
| Solvent | Type | D-1/D-4 | D-1/D-4 | D-1/D-4 | D-1/D-4 | D-1/D-4 | D-1/D-4 | D-1/D-4 |
| [D] | Amount | 4280/1830 | 4280/1830 | 4280/1830 | 4280/1830 | 4280/1830 | 4280/1830 | 4280/1830 |
| | (part(s) | | | | | | | |
| | by | | | | | | | |
| | mass) | | | | | | | |

| | | Comparative | Comparative | Comparative | Comparative | Comparative |
|---|---|---|---|---|---|---|
| | | Example 14 | Example 15 | Example 16 | Example 17 | Example 18 |
| Radiation-sensitive | | CJ-14 | CJ-15 | CJ-16 | CJ-17 | CJ-18 |
| resin composition | | | | | | |
| Resin [A] | Type | A-12 | A-12 | A-12 | A-12 | A-12 |
| | Amount | 100 | 100 | 100 | 100 | 100 |
| | (part(s) | | | | | |
| | by | | | | | |
| | mass) | | | | | |
| Acid- | Type | B-1 | B-1 | B-1 | B-1 | B-1 |
| generating | Amount | 16.0 | 16.0 | 16.0 | 16.0 | 16.0 |
| agent [B] | (part(s) | | | | | |
| | by | | | | | |
| | mass) | | | | | |
| Acid | Type | cc-4 | cc-5 | cc-6 | cc-7 | cc-10 |
| diffusion | Amount | 10.0 | 10.0 | 10.0 | 10.0 | 10.0 |
| control | (part(s) | | | | | |
| agent [C] | by | | | | | |
| | mass) | | | | | |
| Resin [E] | Type | E-5 | E-5 | E-5 | E-5 | E-5 |
| | Amount | 3.0 | 3.0 | 3.0 | 3.0 | 3.0 |
| | (part(s) | | | | | |
| | by | | | | | |
| | mass) | | | | | |
| | Type | D-1/D-4 | D-1/D-4 | D-1/D-4 | D-1/D-4 | D-1/D-4 |
| Solvent | Amount | 4280/1830 | 4280/1830 | 4280/1830 | 4280/1830 | 4280/1830 |
| [D] | (part(s) | | | | | |
| | by | | | | | |
| | mass) | | | | | |

### <Formation of Resist Pattern by Use of Positive-Type Radiation-Sensitive Resin Composition for Exposure to EUV>

An underlayer-forming composition (“ARC66,” product of Brewer Science, Inc.) was applied onto a 12-inch silicon wafer by means of a spin coater (“CLEAN TRACK ACT12,” product of Tokyo Electron Limited), and heated at 205° C. for 60 seconds, to thereby form an underlayer film having an average thickness of 105 nm. Onto the thus-formed underlayer film, each of the above-prepared radiation-sensitive resin compositions for exposure to EUV was applied by means of the aforementioned spin coater, and heated at 130° C. for 60 seconds for PB. Subsequently, the PB product was cooled at 23° C. for 30 seconds, to thereby form a resist film having an average thickness of 55 nm. Then, the resist film was irradiated with light by means of an EUV exposure device (“NXE3300,” product of ASML, NA=0.33, lighting condition: Conventional s=0.89, and mask: imecDEFECT32FFR02. After the light exposure, the resist film was subjected to PEB at 120° C. for 60 seconds. Then, alkali development of the resist film was performed by use of 2.38 mass % aqueous TMAH alkaline developer, and washing with water was conducted after development, followed by drying, to thereby form a positive-type resist pattern (32 nm line-and-space pattern).

### <Evaluation>

The resist patterns formed by use of the aforementioned positive-type radiation-sensitive resin compositions for exposure to EUV were evaluated in terms of sensitivity, LWR performance, and storage stability through the following procedures. Table 9 shows the results. The measurement of the resist pattern was conducted by means of a scanning electron microscope (“CG-5000,” product of Hitachi High-Tech Corporation).

### [Sensitivity]

In formation of a resist pattern by use of a positive-type radiation-sensitive resin composition for exposure to EUV, a dose which can form a 32-nm line-and-space pattern was employed as an optimum dose, serving as a sensitivity (mJ/cm²). A sensitivity of 25 mJ/cm² or lower was evaluated as “good,” and a sensitivity in excess of 25 mJ/cm² was evaluated as “bad.”

### [Lwr Performance]

A resist pattern was formed by modifying the mask size such that a 32 nm line-and-space pattern was formed through irradiation at an optimum dose determined in the aforementioned sensitivity evaluation. The thus-formed resist pattern was observed from above under the aforementioned scanning electron microscope. The line width was measured from above at 500 points, and variation in width (3σ) was determined from the distribution of the width measurements. The 3σ value was employed as an LWR index (nm). Regarding LWR performance, the smaller the 3σ value, the smaller the roughness in line (i.e., the more excellent the LWR performance). LWR performance was evaluated as “good” when the 36 was 2.5 nm or less, and as “bad” when the 36 was in excess of 2.5 nm.

### [Storage Stability]

The aforementioned positive-type radiation-sensitive resin compositions for exposure to EUV were stored at 35° C. for 30 days. Thereafter, the optimum dose which can form a 32-nm line-and-space pattern (i.e., sensitivity) was determined again. When a drop in sensitivity after storage for 30 days was 0% to 1.0%, storage stability was evaluated as “A” (very good). When the drop in sensitivity was greater than 1.0% and 2.0% or smaller, storage stability was evaluated as “B” (good). When the drop in sensitivity was greater than 2.0%, storage stability was evaluated as “C” (bad).

TABLE 9

| | Radiation- | | | |
|---|---|---|---|---|
| | sensitive resin | Sensitivity | LWR | Storage |
| | composition | (mJ/cm²) | (nm) | stability |
| Example 60 | J-60 | 22 | 2.2 | A |
| Example 61 | J-61 | 20 | 2.1 | A |
| Example 62 | J-62 | 21 | 2.3 | A |
| Example 63 | J-63 | 23 | 2.3 | A |
| Example 64 | J-64 | 22 | 2.1 | A |
| Example 65 | J-65 | 21 | 2.0 | A |
| Example 66 | J-66 | 20 | 2.3 | A |
| Example 67 | J-67 | 22 | 2.2 | A |
| Example 68 | J-68 | 19 | 2.1 | A |
| Example 69 | J-69 | 23 | 2.3 | A |
| Example 70 | J-70 | 19 | 2.3 | A |
| Example 71 | J-71 | 19 | 2.3 | A |
| Example 72 | J-72 | 24 | 1.9 | A |
| Comparative | CJ-13 | 26 | 3.3 | A |
| Example 13 | | | | |
| Comparative | CJ-14 | 27 | 3.6 | C |
| Example 14 | | | | |
| Comparative | CJ-15 | 31 | 4.0 | C |
| Example 15 | | | | |
| Comparative | CJ-16 | 32 | 3.7 | C |
| Example 16 | | | | |
| Comparative | CJ-17 | 30 | 3.5 | C |
| Example 17 | | | | |
| Comparative | CJ-18 | 29 | 3.8 | B |
| Example 18 | | | | |

As is clear from Table 9, the radiation-sensitive resin compositions of Examples 60 to 72 were found to exhibit suitable sensitivity, LWR performance, and storage stability, when they were employed in exposure to EUV. In contrast, the radiation-sensitive resin composition of Comparative Example 13 was found to exhibit sensitivity and LWR performance, inferior to those obtained in Examples 60 to 72. Also, the radiation-sensitive resin compositions of Comparative Examples 14 to 18 were found to exhibit sensitivity, LWR performance, and storage stability, inferior to those obtained in Examples 60 to 72.

<Preparation of positive-type radiation-sensitive resin composition for exposure to ArF light, formation of resist pattern by use of the composition, and evaluation of the resist pattern>

### Example 73

(A-1) serving as the resin [A] (100 parts by mass), (B-7) serving as the radiation-sensitive acid-generating agent [B](10.0 parts by mass), (C-1) serving as the acid diffusion control agent [C] (9.0 parts by mass), (E-2) serving as the high-fluorine content resin [E] (5.0 parts by mass (solid content)), and a (D-1)/(D-2)/(D-3) mixed solvent serving as the solvent [D] (3,230 parts by mass (2,240/960/30 (parts by mass)) were mixed together, and the mixture was filtered through a membrane filter (pore size: 0.2 μm), to thereby prepare a positive-type radiation-sensitive resin composition for exposure to ArF light (J-73).

An underlayer-forming composition (“ARC66,” product of Brewer Science, Inc.) was applied onto a 12-inch silicon wafer by means of a spin coater (“CLEAN TRACK ACT12,” product of Tokyo Electron Limited), and heated at 205° C. for 60 seconds, to thereby form an underlayer film having an average thickness of 100 nm. Onto the thus-formed underlayer film, the above-prepared positive-type radiation-sensitive resin composition for exposure to ArF light (J-73) was applied by means of the aforementioned spin coater, and heated at 100° C. for 60 seconds for PB (pre-baking). Subsequently, the PB product was cooled at 23° C. for 30 seconds, to thereby form a resist film having an average thickness of 90 nm. Then, the resist film was irradiated with light by means of an ArF excimer laser liquid immersion light exposure device (“TWINSCAN XT-1900i,” product of ASML, NA=1.35, lighting condition: Annular (6=0.8/0.6) through a mask pattern (50 nm line-and-space). After the light exposure, the resist film was subjected to PEB at 100° C. for 60 seconds. Then, alkali development of the resist film was performed by use of 2.38 mass % aqueous TMAH alkaline developer, and washing with water was conducted after development, followed by drying, to thereby form a positive-type resist pattern (50 nm line-and-space pattern).

The resist pattern formed by use of the aforementioned positive-type radiation-sensitive resin composition for exposure to ArF light was evaluated in terms of sensitivity, LWR performance, and storage stability, in a manner similar to that of evaluation of the resist pattern formed by use of the aforementioned positive-type radiation-sensitive resin composition for exposure to EUV. As a result, the radiation-sensitive resin composition of Example 73 was found to provide suitable sensitivity, LWR performance, and storage stability, even in formation of a positive-type resist pattern through exposure to ArF light.

### <Preparation of Negative-Type Radiation-Sensitive Resin Composition for Exposure to EUV, Formation of Resist Pattern by Use of the Composition, and Evaluation of the Resist Pattern>

### Example 74

(A-15) serving as the resin [A] (100 parts by mass), (B-1) serving as the radiation-sensitive acid-generating agent [B] (15.0 parts by mass), (C-16) serving as the acid diffusion control agent [C] (10.0 parts by mass), (E-5) serving as the high-fluorine content resin [E] (1.0 part by mass (solid content)), and a (D-1)/(D-4) mixed solvent serving as the solvent [D] (6,110 parts by mass (4,280/1,830 (parts by mass)) were mixed together, and the mixture was filtered through a membrane filter (pore size: 0.2 μm), to thereby prepare a negative-type radiation-sensitive resin composition for exposure to EUV (J-74).

An underlayer-forming composition (“ARC66,” product of Brewer Science, Inc.) was applied onto a 12-inch silicon wafer by means of a spin coater (“CLEAN TRACK ACT12,” product of Tokyo Electron Limited), and heated at 205° C. for 60 seconds, to thereby form an underlayer film having an average thickness of 105 nm. Onto the thus-formed underlayer film, each of the above-prepared negative-type radiation-sensitive resin compositions for exposure to EUV (J-74) was applied by means of the aforementioned spin coater, and heated at 130° C. for 60 seconds for PB. Subsequently, the PB product was cooled at 23° C. for 30 seconds, to thereby form a resist film having an average thickness of 55 nm. Then, the resist film was irradiated with laser light by means of an EUV exposure device (“NXE3300,” product of ASML, NA=0.33, lighting condition: Conventional s=0.89, and mask: imecDEFECT32FFRO2). After the light exposure, the resist film was subjected to PEB at 120° C. for 60 seconds. Then, organic solvent development of the aforementioned resist film was performed by use of n-butyl acetate serving as an organic solvent developer, and drying was conducted, to thereby form a negative-type resist pattern (40 nm hole and 105 nm pitch).

The resist pattern formed by use of the aforementioned negative-type radiation-sensitive resin composition for exposure to EUV was evaluated in terms of sensitivity, CDU performance, and storage stability, in a manner similar to that of evaluation of the resist pattern formed by use of the aforementioned negative-type radiation-sensitive resin composition for exposure to ArF light. As a result, the radiation-sensitive resin composition of Example 74 was found to provide suitable sensitivity, CDU performance, and storage stability, even in formation of a negative-type resist pattern through exposure to EUV.

According to the aforementioned radiation-sensitive resin composition and resist pattern formation method, suitable sensitivity to exposure light and excellent LWR performance and CDU performance can be provided. Thus, the embodiments of the invention can be suitably applied to processing of semiconductor devices and the like, which conceivably require further process shrinkage.

What is claimed is:

1. A radiation-sensitive composition comprising: a polymer comprising an acid-releasable group; and a compound (Q) represented by formula (1): 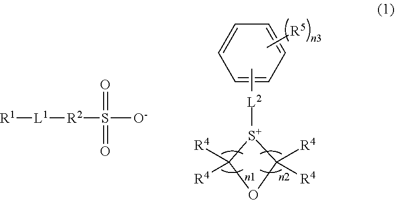
wherein L¹ represents an ester group, —CO—NR³—, a (thio)ether group, or a sulfonyl group; when L¹ is an ester group, a (thio)ether group, or a sulfonyl group, R¹, R², and R³ satisfy condition (i) or (ii); when L¹ is —CO—NR³—, R¹, R², and R³ satisfy condition (i), (ii), or (iii); R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom, and optionally two R⁵s taken together represent an alicyclic structure together with the carbon atom(s) between the two R⁵s; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another:
(i) R¹ represents a C1 to C20 monovalent organic group which bound to L¹ via a carbon atom included in R¹; R² represents a substituted or unsubstituted divalent hydrocarbon group, comprising no fluorine atom; and R³ represents a hydrogen atom or a monovalent hydrocarbon group;
(ii) R¹ and R² taken together represent a group comprising an aliphatic heterocyclic structure together with L¹ to which R¹ and R² are bound, provided that R² comprises no fluorine atom; and R³ represents a hydrogen atom or a monovalent hydrocarbon group; and
(iii) R¹ represents a C1 to C20 monovalent organic group which bound to L¹ via a carbon atom included in R¹; R² and R³ taken together represent an aliphatic heterocyclic structure together with L¹ to which R² and R³ are bound, the aliphatic heterocyclic structure comprising no fluorine atom.

2. The radiation-sensitive composition according to claim 1, wherein R² is a substituted or unsubstituted divalent chain hydrocarbon group or a substituted or unsubstituted divalent alicyclic hydrocarbon group, or R² and R³ taken together represent an aliphatic heterocyclic structure together with L¹.

3. The radiation-sensitive composition according to claim 1, wherein R¹ is a substituted or unsubstituted monovalent chain hydrocarbon group, a monovalent group having an alicyclic hydrocarbon structure, or a monovalent group having an aliphatic heterocyclic structure, each group being bound to L¹ via a carbon atom included in R¹.

4. The radiation-sensitive composition according to claim 1, wherein
R² is a substituted or unsubstituted divalent chain hydrocarbon group or a substituted or unsubstituted divalent alicyclic hydrocarbon group, or R² and R³ taken together represent an aliphatic heterocyclic structure together with L¹; and
R¹ is a substituted or unsubstituted monovalent chain hydrocarbon group, a monovalent group having an alicyclic hydrocarbon structure, or a monovalent group having an aliphatic heterocyclic structure, each group being bound to L¹ via a carbon atom included in R¹.

5. The radiation-sensitive composition according to claim 1, wherein L² is a single bond.

6. The radiation-sensitive composition according to claim 1, further comprising a compound (B) which generates, in the composition, an acid having an acidity higher than an acid generated by the compound (Q) through irradiation with a radiation.

7. The radiation-sensitive composition according to claim 6, wherein the compound (B) is a compound represented by formula (2): 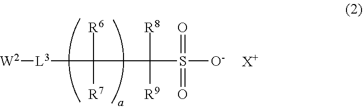
wherein W² represents a C3 to C40 monovalent organic group; L³ represents a single bond or a divalent bonding group;
each of R⁶, R⁷, R³, and R⁹ independently represents a hydrogen atom, a C1 to C10 hydrocarbon group, a fluorine atom, or a C1 to C10 fluoroalkyl group; a is an integer of 0 to 8; when a is ≥2, a plurality of R⁶s and R⁷ s are identical to or different from one another; at least one member of (a×2+2) groups of R⁶, R⁷, R³, and R⁹ in the formula is a fluorine atom or a fluoroalkyl group; and X⁺ represents a monovalent cation.

8. A pattern formation method, comprising:
forming a resist film by applying the radiation-sensitive composition according to claim onto a substrate,
exposing the resist film to a radiation, and
developing the radiation-exposed resist film.

9. The pattern formation method according to claim 8, wherein in the developing, the radiation-exposed resist film is developed with an organic solvent developer.

10. A light-degradable base represented by formula (1): 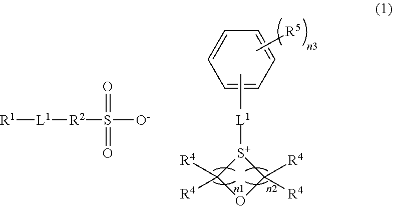
wherein L¹ represents an ester group, —CO—NR³—, a (thio) ether group, or a sulfonyl group; when L¹ is an ester group, a (thio)ether group, or a sulfonyl group, R¹, R², and R³ satisfy the condition (i) or (ii); when L¹ is —CO—NR³—, R¹, R², and R³ satisfy the condition (i), (ii), or (iii); R⁴ represents a hydrogen atom, a substituted or unsubstituted C1 to C20 monovalent hydrocarbon group, a halogen atom, a hydroxy group, or a nitro group; R⁵ represents a C1 to C20 monovalent hydrocarbon group, a C1 to C20 monovalent halogenated hydrocarbon group, or a halogen atom, and optionally two R⁵s taken together represent an alicyclic structure together with the carbon atom(s) between the two R⁵s; L² represents a single bond or a divalent linking group; each of n1 and n2 is independently an integer of 1 to 4; n3 is an integer of 0 to 5; when n3 is ≥2, a plurality of R⁵s are identical to or different from one another; and a plurality of R⁴s are identical to or different from one another:
(i) R¹ represents a C1 to C20 monovalent organic group which bound to L¹ via a carbon atom included in R¹; R² represents a substituted or unsubstituted divalent hydrocarbon group, comprising no fluorine atom; and R³ represents a hydrogen atom or a monovalent hydrocarbon group;
(ii) R¹ and R² taken together represent an aliphatic heterocyclic structure together with L¹ to which R¹ and R² are bound, provided that R² comprises no fluorine atom; and R³ represents a hydrogen atom or a monovalent hydrocarbon group; and
(iii) R¹ represents a C1 to C20 monovalent organic group which bound to L¹ via a carbon atom included in R¹; R² and R³ taken together represent an aliphatic heterocyclic structure together with L¹ to which R² and R³ are bound, the aliphatic heterocyclic structure comprising no fluorine atom.
